SEQUENCE LISTING (29 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 3 - DNA, 396 bp, Homo sapiens
atgtggctccagagcctgctactcctggggacggtggcctgcagcatctcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtga

SEQ ID NO: 4 - DNA, 1847 bp, Artificial Sequence
  source: /note=“Description of Artificial Sequence Synthetic polynucleotide”
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacaagaaatgcggatctcgaagccgcacctgcggtcgatatcgatccagtgctacctgtgcctgctcctgaactcgcacttcctcacggaggccggtatacacgtcttcatcctgggctgcttctcggcggggctgccgaagacggaggcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtgagggcccggcgcgccgaattcgcggatatcggttaacggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcata

SEQ ID NO: 5 - protein, 162 aa, Artificial Sequence; identical to SEQ ID NO: 1
  source: /note=“Description of Artificial Sequence Synthetic polypeptide”

SEQ ID NO: 6 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 7 - protein, 200 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 8 - DNA, 804 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 9 - DNA, 600 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggc

SEQ ID NO: 10 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 11 - DNA, 2140 bp, Artificial Sequence
  source: /note=“Description of Artificial Sequence Synthetic polynucleotide”
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgctagcaagaaatggccccgaggcgggcgcgaggctgccggaccctcggtctcccggcgctgctactgctcctgctgctccggccgccggcgacgcggggcatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacgacggcgggggtgaccccgcagccggagagcctctccccgtcgggaaaggagcccgccgcgtcgtcgcccagctcgaacaacacggcggccacaactgcagcgatcgtcccgggctcccagctgatgccgtcgaagtcgccgtccacgggaaccacggagatcagcagtcatgagtcctcccacggcaccccctcgcaaacgacggccaagaactgggaactcacggcgtccgcctcccaccagccgccgggggtgtatccgcaaggccacagcgacaccacggtggcgatctccacgtccacggtcctgctgtgtgggctgagcgcggtgtcgctcctggcgtgctacctcaagtcgaggcagactcccccgctggccagcgttgagatggaggccatggaggctctgccggtgacgtgggggaccagcagcagggatgaggacttggagaactgctcgcaccacctataatgagaattcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcata

SEQ ID NO: 12 - protein, 267 aa, Artificial Sequence; identical to SEQ ID NO: 6
  source: /note=“Description of Artificial Sequence Synthetic polypeptide”

SEQ ID NO: 13 - DNA, 1971 bp, Artificial Sequence
  source: /note=“Description of Artificial Sequence Synthetic polynucleotide”
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgctagcaagaaatggccccgaggcgggcgcgaggctgccggaccctcggtctcccggcgctgctactgctcctgctgctccggccgccggcgacgcggggcatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacgacggcgggggtgaccccgcagccggagagcctctccccgtcgggaaaggagcccgccgcgtcgtcgcccagctcgaacaacacggcggccacaactgcagcgatcgtcccgggctcccagctgatgccgtcgaagtcgccgtccacgggaaccacggagatcagcagtcatgagtcctcccacggcaccccctcgcaaacgacggccaagaactgggaactcacggcgtccgcctcccaccagccgccgggggtgtatccgcaaggccacagcgacaccacgtaatgagaattcgcggatatcggttaacggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcata

SEQ ID NO: 14 - protein, 205 aa, Artificial Sequence
  source: /note=“Description of Artificial Sequence Synthetic polypeptide”
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 15 - protein, 8 aa, Homo sapiens
PQGHSDTT

SEQ ID NO: 16 - protein, 7 aa, Homo sapiens
PQGHSDT

SEQ ID NO: 17 - protein, 6 aa, Homo sapiens
PQGHSD

SEQ ID NO: 18 - protein, 5 aa, Homo sapiens
PQGHS

SEQ ID NO: 19 - protein, 4 aa, Homo sapiens
PQGH

SEQ ID NO: 20 - protein, 3 aa, Homo sapiens
PQG

SEQ ID NO: 21 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 22 - protein, 19 aa, Homo sapiens
NWELTASASHQPPGVYPQG

SEQ ID NO: 23 - protein, 17 aa, Homo sapiens
ITCPPPMSVEHADIWVK

SEQ ID NO: 24 - protein, 31 aa, Homo sapiens
ITCPPPMSVEHADIWVKSYSLYSRERYICNS

SEQ ID NO: 25 - protein, 4 aa, Artificial Sequence
  source: /note=“Description of Artificial Sequence Synthetic peptide”
  MOD_RES (2)..(3): Any amino acid
RXXR

SEQ ID NO: 26 - protein, 6 aa, Artificial Sequence
  source: /note=“Description of Artificial Sequence Synthetic peptide”
  MOD_RES (1)..(2): Any hydrophobic amino acid
  MOD_RES (5)..(6): Any non-acidic amino acid
XXPRXX

SEQ ID NO: 27 - protein, 436 aa, Artificial Sequence
  source: /note=“Description of Artificial Sequence Synthetic polypeptide”
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 431 aa, Artificial Sequence
  source: /note=“Description of Artificial Sequence Synthetic polypeptide”
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 159 aa, Artificial Sequence
  source: /note=“Description of Artificial Sequence Synthetic polypeptide”
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELADIG

(19) United States
(12) Patent Application Publication
(10) Pub. No.: US 2021/0244821 A1
(43) Pub. Date: Aug. 12, 2021

(54) CHO CELL EXPRESSED HET IL-15

(71) Applicant: NOVARTIS AG, Basel (CH)

(72) Inventors: Richard GABRIEL, Sierentz (FR); Thomas JOSTOCK, Neuenburg am Rhein (DE); Justyna JOZEFCZUK, Binningen (CH); Urs Ulrich LOHRIG, Kundl (AT); Alexandre Aurélien PONS, Oberperfuss (AT)

(21) Appl. No.: 17/161,929

(22) Filed: Jan. 29, 2021

Related U.S. Application Data
(60) Provisional application No. 62/970,485, filed on Feb. 5, 2020.

(51) Int. Cl. A61K 47/54; A61P 35/04; A61K 38/20; A61K 38/17

(52) U.S. Cl. CPC A61K 47/549; A61K 38/1793; A61K 38/2086; A61P 35/04

(57) ABSTRACT

The present invention relates to IL-15/IL-15Rα heterodimer produced in a CHO cell line, and method of producing the heterodimer and method of treatment using the heterodimer.

### RELATED APPLICATIONS

The present disclosure claims priority to U.S. Patent Application 62/970,485 filed Feb. 5, 2020 which is incorporated by reference herein in its entirety.

### FIELD

The present disclosure relates to polypeptide complex comprising a human interleukin 15 (IL-15) polypeptide and a human interleukin 15 receptor alpha (IL-15Rα) polypeptide having unique glycosylation profile, and methods of producing such polypeptide.

### SEQUENCE LISTING

The instant application contains a Sequence Listing which has been submitted electronically in ASCII format and is hereby incorporated by reference in its entirety. Said ASCII copy, created on Jan. 27, 2021, is named PAT058680-US—NP-Sequencelisting.txt and is 37,833 bytes in size.

### BACKGROUND

The cytokine, interleukin-15 (IL-15), is a member of the four alpha-helix bundle family of lymphokines produced by many cells in the body. IL-15 plays a pivotal role in modulating the activity of both the innate and adaptive immune system, e.g., maintenance of the memory T-cell response to invading pathogens, inhibition of apoptosis, activation of dendritic cells, and induction of Natural Killer (NK) cell proliferation and cytotoxic activity.

The IL-15 receptor consists of three polypeptides, the type-specific IL-15 receptor alpha (“IL-15Rα”), the IL-2/IL-15 receptor beta (or CD122) (“β”), and the common gamma chain (or CD132) (“γ”) that is shared by multiple cytokine receptors. IL-15Rα is thought to be expressed by a wide variety of cell types, but not necessarily in conjunction with β and γ. IL-15 signaling has been shown to occur through the heterodimeric complex of IL-15Rα, β, and γ; through the heterodimeric complex of β and γ, or through a subunit, IL-15RX, found on mast cells.

IL-15 is a soluble protein, but endogenous IL-15 is not readily detectable in serum or body fluids as it occurs predominantly as a membrane-bound form that is expressed or acquired by several types of accessory cells. For instance, although IL-15 mRNA is detected in cells of both hematopoietic and non-hematopoietic lineage, T cells do not produce IL-15. Instead, IL-15 binds to the IL-15Rα, forming cell-surface complexes on T cells. IL-15 specifically binds to the IL-15Rα with high affinity via the “sushi domain” in exon 2 of the extracellular domain of the receptor. After trans-endosomal recycling and migration back to the cell surface, these IL-15 complexes acquire the property to activate bystander cells expressing the IL-15R βγ low-affinity receptor complex, inducing IL-15-mediated signaling via the Jak/Stat pathway. A wild type soluble form of IL-15Rα (“sIL-15Rα”), which is cleaved at a cleavage site in the extracellular domain immediately distal to the transmembrane domain of the receptor has been observed. Tumor necrosis factor-alpha-converting enzyme (TACE/ADAM17) has been implicated as a protease involved in this process.

Based on its multifaceted role in the immune system, various therapies designed to modulate IL-15-mediated function have been explored. Recent reports suggest that IL-15, when complexed with the sIL-15Rα, or the sushi domain, maintains its immune enhancing function. Recombinant IL-15 and IL-15/IL-15Rα complexes have been shown to promote to different degrees the expansion of memory CD8 T cells and NK cells and enhance tumor rejection in various preclinical models. Furthermore, tumor targeting of IL-15 or IL-15/IL-15Rα complex containing constructs in mouse models, resulted in improved anti-tumor responses in either immunocompetent animals transplanted with syngeneic tumors or in T- and B cell-deficient SCID mice (retaining NK cells) injected with human tumor cell lines. Enhanced anti-tumor activity is thought to be dependent on increased half-life of the IL-15-containing moiety as well as the trans-presentation of IL-15 on the surface of tumor cells leading to enhanced NK and/or CD8 cytotoxic T cell expansion within the tumor. As such, tumor cells engineered to express IL-15 were also reported to promote rejection of established tumors by enhancing T cell and NK cell recruitment, proliferation and function (Zhang et al, (2009) PNAS USA. 106:7513-7518; Munger et al, (1995) Cell Immunol. 165(2):289-293; Evans et al, (1997) Cell Immunol. 179(1):66-73; Klebanoff et al, (2004) PNAS USA. 101(7):1969-74; Sneller et al, (2011) Blood. 118(26):6845-6848; Zhang et al, (2012) J. Immunol. 188(12):6156-6164).

It is well recognized that the biological activity of a protein containing oligosaccharide chains, known as a glycoprotein, is dependent upon not only the integral structure of the protein, but also the properties of the oligosaccharide covalently attached to the protein. Glycosylation can effect solubility, resistance to proteolytic attack and thermal inactivation, quaternary structure, activity, targeting, antigenicity, functional activity, and half-life of the protein. Mammalian glycosylation patterns in general are described in Fukuda et al. (1994), Molecular Glycobiology, IRL Press, New York, incorporated herein by reference. Sialic acid N-Acetylneuraminic acid (NANA) is a major constituent of N- and O-linked glycans. Sialic acid has been shown to be important in sustaining the half-life of protein therapeutics. It is known that desialylated or under-sialylated glycoproteins have significantly reduced half-life in plasma. Thus, it is advantageous to produce IL-15/IL-15Rα complex with unique glycosylation profile.

### SUMMARY OF THE INVENTION

Disclosed herein are compositions of IL-15/IL-15Rα heterodimer with unique glycosylation profile. N-Acetylneuraminic acid (NANA) is a major constituent of N- and O-linked glycans. NANA is the predominant form of neuraminic acids within glycosylation events of proteins in humans, while other mammals may also include other derivatives such as N-glycolylneuraminic acid (NGNA). NANA can be linked in several ways to the core structures of N- and O-glycans. Dominantly, α(2, 3) and α(2, 6) linkages to the subsequent saccharide can be found, although other linkages such as α(2, 8) exist. Human cells, e.g. Human embryonic kidney (HEK) cells, mainly produce the α(2, 6) linked sialylation, whereas many production cell lines, e.g. mammalian cell lines such as the Chinese hamster ovary (CHO) cell line, produce the α(2, 3) linked sialylation.

In CHO cells, the enzyme responsible for producing alpha(2, 6) linked NANA extensions to core glycan structures (Beta-galactoside alpha-2,6-sialyltransferase 1) has been reported as inactive or not-expressed in CHO cells (see e.g. Chung et al. (2017) Biotechnol. J. 12:1600502)—although the gene for the enzyme itself is present in C. griseus. The present invention is based on the unexpected discovery that the CHO cell produced IL-15/IL-15Rα complex has different glycosylation patterns compared to the IL-15/IL-15Rα complex produced by human cell lines. In addition, alpha(2, 6) linkage type glycan was observed in CHO cell produced IL-15/IL-15Rα complex. This glycosylation pattern is unique and surprising since it is usually observed in proteins expressed in human cell lines. It provides direct benefits, for example, less immunogenicity, compared to the expected CHO pattern by being closer to the human glycosylation forms. For example, non-human, potentially immunogenic glycoepitops such as N-glycolylneuraminic acid is essentially absent in the CHO cell produced IL-15/IL-15Rα. In addition, the glycosylation may affect the half-life of cytokines in vivo and their districution, and thus affect the therapeutic efficacy of the glycosylated IL-15/IL-15Rα complex.

On the other hand, HEK cell derived IL-15/IL-15Rα complexes are not considered optimal for further development due to low process robustness, low yields, usage of animal-derived raw materials in the production process, complex analytical characterization with limited resolution, and existence of a splice variant in IL-15Rα recently detected. The IL-15Rα splice variant may cause toxicity. In addition, the presence of additional species makes it difficult to determine the accurate amount of active IL-15/IL-15Rα complex being administered to the patient, and affect the potency of the drug. Susceptibility to viral contamination is also considered a potential risk when using HEK cells to produce recombinant biopharmaceuticals. Thus, CHO cells are considered more optimal for producing IL-15/IL-15Rα complexes because they are safer, not as successible to viral contamination, and have much higher yield.

Accordingly, the present disclosure is directed to a polypeptide complex comprising a human interleukin 15 (IL-15) polypeptide and a human interleukin 15 receptor alpha (IL-15Rα) polypeptide. In some embodiments, the polypeptide complex comprises N-linked glycans comprising FA2G2, FA2G2S1, FA2G2S2, FA3G3S1, FA2F1G2S2, FA3G2S2, and FA3G3S3. In some embodiments, the IL-15 polypeptide has the sequence of SEQ ID NO: 1 or 5, and IL-15Rα has the sequence of SEQ ID NO: 6, 7, 10, 12, 14 or 21.

In some embodiments, the N-linked glycans comprise at least about 10%, 12.5%, 15%, 17.5%, 20% or 22.5% of FA2G2S1. In some embodiments, the N-linked glycans comprise at least about 10%, 11%, 12%, 13%, 14%, 15%, 16%, 17%, 18%, 19%, 20%, 21%, 22%, 23%, 24% or 25% of FA2G2S1.

In some embodiments, the N-linked glycans comprise at least about 10%, 20%, 30%, or 40% of FA2G2S2. In some embodiments, the N-linked glycans comprise at least about 10%, 12.5%, 15%, 17.5%, 20%, 22.5%, 25%, 27.5%, 30%, 32.5%, 35%, 37.5%, or 40% of FA2G2S2.

In some embodiments, the polypeptide complex comprises O-linked glycans. In some embodiments, at least 80%, 85%, 90% or 95% of the glycans is core-1 O-linked glycan.

In some embodiments, the core-1 O-linked glycan is predominantly monosialylated and/or disialylated.

Additionally disclosed herein are polypeptide complexes comprising a human interleukin 15 (IL-15) polypeptide and a human interleukin 15 receptor alpha (IL-15Rα) polypeptide having both O-linked glycans and N-linked glycans. In some embodiments, the 0-linked glycans of the polypeptide complex has least about 80%, 85%, 90% or 95% of the glycans has core-1 O-linked glycan structures; and the polypeptide complex comprises N-linked glycans comprising FA2G2, FA2G2S1, FA2G2S2, FA3G3S1, FA2F1G2S2, FA3G2S2, and FA3G3S3. In some embodiments, the IL-15 polypeptide has the sequence of SEQ ID NO: 1 or 5, and IL-15Rα has the sequence of SEQ ID NO: 6, 7, 10, 12, 14 or 21.

In some embodiments, the N-linked glycans comprise at least about 10%, 12.5%, 15%, 17.5%, 20% or 22.5% of FA2G2S1. In some embodiments, the N-linked glycans comprise at least 10%, 20%, 30%, or 40% of FA2G2S2. In some embodiments, the core-1 O-linked glycan is predominantly monosialylated and/or disialylated.

Additionally disclosed herein are isolated IL-15/IL-15Rα heterodimers produced in a non-human cell, wherein the IL-15/IL-15Rα heterodimer comprises α(2,6) O-linked sialylation. In some embodiments, the non-human cell is a recombinant Chinese hamster ovary (CHO) cell. In some embodiments, the CHO cell is altered to impair the function of matriptase.

In some embodiments, the isolated IL-15/IL-15Rα heterodimer comprises O-linked glycans, and wherein at least 90% or 95% of the glycans is core-1 O-linked glycan. In some embodiments, about 15% of the 0-glycans has α(2,6)-linked sialylation.

Additionally disclosed herein are pharmaceutical compositions comprising any one of the polypeptide complexes or any one of the isolated IL-15/IL-15Rα heterodimers described herein. In some embodiments, the pharmaceutical composition further comprising a pharmaceutically acceptable carrier.

Additionally disclosed herein are non-human cells comprising nucleic acids encoding a human interleukin 15 (IL-15) polypeptide and a human interleukin 15 receptor alpha (IL-15Rα) polypeptide, wherein the IL-15 and IL-15Rα expressed by the cell form a heterodimer, and wherein the heterodimer comprises α(2,6)-linked sialylation.

Additionally disclosed herein are methods of treating cancer, comprising administering to a subject in need of the pharmaceutical composition of described herein.

Additionally disclosed herein are methods of producing cells that express IL-15/IL-15a heterodimer, comprising (a) providing non-human cells; (b) transfecting the non-human cells with two vectors at the same time, wherein the two vectors comprises a first vector encoding both IL-15Rα and IL-15, and a second vector encoding a portion of IL-15Rα and culturing the transfected cells; (c) transfecting the cells from step b) with a third vector encoding IL-15 and culturing the transfected cells; and (d) isolating individual clones that express IL-15/IL-15a heterodimer.

In some embodiments, the non-human cell is a recombinant Chinese hamster ovary (CHO) cell. In some embodiments, the CHO cell is modified to impair the function of the matriptase gene. In some embodiments, the IL-15Rα has the sequence of SEQ ID NO: 12. In some embodiments, the IL-15 has the sequence of SEQ ID NO: 5. In some embodiments, the portion of IL-15 Rα is a soluble portion of IL-15Rα. In some embodiments, the soluble portion of IL-15Rα has the sequence of SEQ ID NO:10.

Additionally disclosed herein are methods of producing IL-15/IL-15 Rα heterodimer, comprising (a) culturing the cells produced above under a condition that allow for expression of a IL-15/IL-15Rα heterodimer and secretion of the IL-15/IL-15Rα heterodimer, and (b) isolating the IL-15/IL-15a heterodimer from the cell culture.

Additional disclosed herein are polypeptide complexes comprising a human interleukin 15 (IL-15) polypeptide and a human interleukin 15 receptor alpha (IL-15Rα) polypeptide, wherein the polypeptide complex is produced by a recombinant Chinese hamster ovary (CHO) cell, and wherein the polypeptide complex does not have a IL-15Rα chain splicing variant.

In some embodiments, the IL-15Rα chain splicing variant comprise 159 residues spanning from I1 to G159 In some embodiments, the CHO cell is altered to impair the function of matriptase.

### BRIEF DESCRIPTION OF THE DRAWINGS

FIGS. 1A-1C show vector maps of pBW1697, pBW1703 and pBW1916. Vector pBW1697 (A) includes genes for expression of the IL-15 and IL-15Rα; pBW1703 (B) includes genes for expression of IL-15Rα and (C) pBW1916 includes genes for expression of IL-15. All three plasmids include SwaI site used for linearization of the vector.

FIGS. 2A-2C show Northern Blot analysis of cells producing IL-15/IL-15Rα (C009) at the beginning and end of stability study. IL-15 (A), IL-15 own SP with propeptide (B) and IL-15Rα (C) mRNA expression of the cell was analyzed by Northern blot. A 0.5-10 kb RNA ladder was loaded into lane M, and control RNA from parental CHO-MaKo cells into lane P. (A) The expected 1 kb band size of IL-15 mRNA was detected. (B) The expected 1 kb band size of IL-15 own SP with propeptide mRNA for was detected. (C) The expected 1 kb band size of IL-15Rα mRNA was detected. No signal was detected for parental cells.

FIGS. 3A-3C show Southern Blot analysis of cells producing IL-15/IL-15Rα (C009) at the beginning and end of stability study. Genomic DNA from the cell was digested with restriction enzyme VA and analyzed by Southern blot, using a probe So465 targeting the IL-15 gene sequence (A), probe So466 targeting the IL-15 own SP+propeptide sequence (B) and probe So467 targeting the IL-15Rα gene sequence (C). A DNA ladder (Molecular DNA Marker VII, Roche) was loaded into lane M. Lanes 1-3 contained MfeI-digested genomic DNA from parental CHO-MaKo cells, without (lane 4) or with spiked MfeI-digested pBW1697 (lane 1), pBW1703 (lane 2) and pBW1916 (lane 3) vector DNA at 5 copies per genome. (A) The expected band size of 1.9 kb for the IL-15 fragment is detected from pBW1697 (lane 1), from pBW1916 (lane 3) and at beginning and end of stability (lane 5 and 6). (B) The expected band size of 1.9 kb for the IL-15 own SP+propeptide fragment is detected from pBW1697 (lane 1), no band is detected at beginning and end of stability (lane 5 and 6). (C) The expected band size of 2.5 kb for the IL-15Rα fragment (IL-15Rα FL) is detected from pBW1697 (lane 1), band size of 2 kb (IL-15Rα sol) is detected from pBW1703 (lane 2) and at beginning and end of stability (lane 5 and 6). No signal could be detected for parental cells.

FIG. 4 shows Transgene copy number of cells producing IL-15/IL-15Rα (C009) at beginning and end of stability. IL-15/own SP (black columns), IL-15Rα/UTR12SP (dark grey columns) and IL-15/UTR12SP (light grey columns) gene copy numbers (per haploid genome) were measured by qPCR for cells producing IL-15/IL-15Rα and CHO-MaKo parental cells.

FIGS. 5A and 5B show a 2,3- and a 2,6-linked sialic acid discrimination. Panel A depicts the ethyl-esterificatin of 6′ sialyllactose, panel B depicts the formation of a lactone for 3′-sialyllactose.

FIG. 6 shows 0-glycan distribution of het IL-15.

FIG. 7 shows core 1 type structure.

FIG. 8 shows core 2 type structure.

FIG. 9 shows MS spectrum of het IL-15.

FIG. 10 shows N-glycan profiles of HEK and CHO produced het IL-15.

FIG. 11 shows N-glycan profiles of the HEK produced het IL-15.

FIG. 12 shows N-glycan profiles of the CHO produced het IL-15.

FIGS. 13A and 13B show nomenclature for individual building blocks of glycans.

FIG. 14 shows dose sescalation and expansion study design

FIG. 15 shows chromatographic profiles of HEK293 batches and CHO batches.

### DETAILED DESCRIPTION

### General Matters

In order that the present invention may be more readily understood, certain terms are defined throughout the detailed description. Unless defined otherwise, all technical and scientific terms used herein have the same meaning as commonly understood by those of ordinary skill in the art to which this invention pertains.

### Terminology

Unless stated otherwise, the following terms and phrases as used herein are intended to have the following meanings:

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The terms “disease” and “disorder” are used interchangeably to refer to a condition, in particular, a pathological condition. In certain embodiments, the terms “disease” and “disorder” are used interchangeably to refer to a disease affected by IL-15 signal transduction and/or a disease affected by the promotion of an immune effector response.

As used herein, the terms “treat”, “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

As used herein, the terms “therapies” and “therapy” can refer to any protocol(s), method(s), compositions, formulations, and/or agent(s) that can be used in the prevention, treatment, management, or amelioration of a disease, e.g., cancer, infectious disease, lymphopenia, and immunodeficiencies, or a symptom associated therewith. In certain embodiments, the terms “therapies” and “therapy” refer to biological therapy, supportive therapy, and/or other therapies useful in treatment, management, prevention, or amelioration of a disease or a symptom associated therewith known to one of skill in the art.

As used herein, the terms “specifically binds,” “specifically recognizes” and analogous terms in the context of a receptor (e.g., native IL-15Rα or IL-15 receptor βγ) and a ligand (e.g., native IL-15) interaction refer to the specific binding or association between the ligand and receptor. Preferably, the ligand has higher affinity for the receptor than for other molecules. In a specific embodiment, the ligand is native IL-15 and the native receptor is IL-15Rα. In another specific embodiment, the ligand is the native IL-15/IL-15Rα complex and the native receptor is the βγ receptor complex. In a further embodiment, the IL-15/IL-15Rα complex binds to the βγ receptor complex and activates IL-15 mediated signal transduction. Ligands that specifically bind a receptor can be identified, for example, by immunoassays, BIAcore™, or other techniques known to those of skill in the art.

As used herein, the term “immunospecifically binds” and “specifically binds” in the context of antibodies refer to molecules that specifically bind to an antigen (e.g., an epitope or an immune complex) and do not specifically bind to another molecule. A molecule that specifically binds to an antigen may bind to other antigens with a lower affinity as determined by, e.g., immunoassays, BIAcore™ or other assays known in the art. In a specific embodiment, molecules that bind to an antigen do not cross-react with other antigens.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Immune effector” or “effector” “function” or “response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring or wild type sequence, or are encoded by a substantially identical nucleotide sequence, and are capable of having one or more activities of the naturally-occurring or wild type sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 70%, preferably at least 80%, more preferably at least 90%, 95%, and even more preferably at least 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available from the NCBI), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to a nucleic acid (SEQ ID NO: 2) molecules of the invention. BLAST protein searches can be performed with the XBLAST program, score=50, word length=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used (available from the NBCI).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “glycan” is a sugar, which can be monomers or polymers of sugar residues, such as at least three sugars, and can be linear or branched (e.g., have an α 1,3 arm and an α 1,6 arm). A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties. The polysaccharide is attached either via the OH group of serine or threonine (0-glycosylated polypeptide) or via the amide group (NH 2) of asparagine (N-glycosylated polypeptide). The glycoprotein may be homologous to the host cell or preferably heterologous to the host cell expressing it, ie foreign, eg, a human protein produced by CHO cells.

The term “glycoconjugate,” as used herein, encompasses all molecules in which at least one sugar moiety is covalently linked to at least one other moiety. The term specifically encompasses all biomolecules with covalently attached sugar moieties, including for example N-linked glycoproteins, O-linked glycoproteins, glycolipids, proteoglycans, etc.

As used herein, the term “glycosylation pattern” refers to the set of glycan structures present on a particular sample. For example, a particular glycoconjugate (e.g., glycoprotein) or set of glycoconjugates (e.g., set of glycoproteins) will have a glycosylation pattern. In some embodiments, reference is made to the glycosylation pattern of cell surface glycans. A glycosylation pattern can be characterized by, for example, the identities of glycans, amounts (absolute or relative) of individual glycans or glycans of particular types, degree of occupancy of glycosylation sites, etc., or combinations of such parameters.

Various aspects of the invention are described in further detail below. Additional definitions are set out throughout the specification.

Glycosylation

The term “glycosylation” refers to the attachment of a polysaccharide to a polypeptide. Preferably, the polysaccharide consists of 2-12 monosaccharides linked together by glycosidic bonds. Glycoproteins can contain O-linked sugar moieties and/or N-linked sugar moieties. The structure and number of sugar moieties attached to a particular glycosylatoin site can be variable. Such sugar moieties may be, for instance, N-acetyl glucosamine, N-acetyl galactosamine, mannose, galactose, glucose, fucose, xylose, glucuronic acid, iduronic acid and/or sialic acids.

The term “N-linked glycosylation” refers to the attachment of a polysaccharide to an asparagine residue of an amino acid chain.

The term “O-linked glycosylation” refers to the attachment of a carbohydrate moiety to a serine or threonine residue of an amino acid chain.

The terms “sugar profile” or “glycosylation profile”, used interchangeably in this application, describe the glycan nature of a glycosylated polypeptide. These properties are preferably the glycosylation site, or the occupancy of the glycosylation site, or the identity, structure, composition or amount of the glycan and/or non-sugar portion of the polypeptide, or the identity and amount of a specific glycoform.

N-Acetylneuraminic acid (NANA) is a major constituent of N- and O-linked glycans. NANA is the predominant form of neuraminic acids within glycosylation events of proteins in humans, while other mammals may also include other derivatives such as N-glycolylneuraminic acid (NGNA). NANA can be linked in several ways to the core structures of N- and O-glycans. Dominantly, α(2, 3) and α(2, 6) linkages to the subsequent saccharide can be found, although other linkages such as α(2, 8) exist. Human cells, e.g. Human embryonic kidney (HEK) cells, mainly produce the α(2, 6) linked sialylation, whereas many production cell lines, such as CHO cell line, produce the α(2, 3) linked sialylation.

In CHO cells, the enzyme responsible for producing alpha(2-6) linked NANA extensions to core glycan structures (Beta-galactoside alpha-2,6-sialyltransferase 1) has been reported as inactive or not-expressed in CHO cells (see e.g. Chung et al. (2017) Biotechnol. J. 12:1600502)—although the gene for the enzyme itself is present in C. griseus. The present invention is based on the unexpected discovery that the CHO cell produced IL-15/IL-15Rα complex has different glycosylation patterns compared to the IL-15/IL-15Rα complex produced by human cell lines. In addition, alpha(2-6) linkage type glycan was observed in CHO cell produced IL-15/IL-15Rα complex. This glycosylation pattern is unique. It may potentially provide direct benefits compared to the expected CHO pattern by being closer to the human glycosylation forms.

In some embodiments, the IL-15/IL-15Rα heterodimer of the present disclosure has one or more of the glycan species as shown in Table 1 below.

TABLE 1
structure and nomenclature of known glycan species.

| | m/z theor. | |
|---|---|---|
| Nomenclature | [M + Na]+ | Structure |
| H1 | 289.1622 | 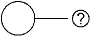 |
| N1 (or T) | 330.1887 | 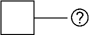 |
| C1 (or Tn) | 534.2885 | 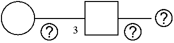 |
| H1S1 | 650.3358 | 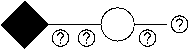 |
| N1S1 (or TS1) | 691.3624 | 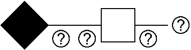 |
| C1F1 (or TnF1) | 708.3777 | 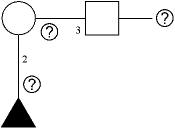 |
| C2 or C1N1 | 779.4148 | 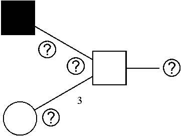 [SMILES: CC1CCC(C)CC(C2CCCCCC2)CC1] |
| | | 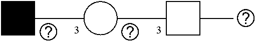 |
| C1S1 (or TnS1) | 895.4621 | 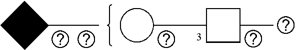 |
| C1N1F1 or C2F1 | 953.5040 | 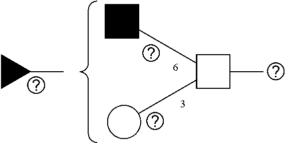 |
| | | 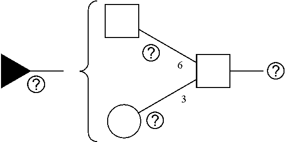 |
| | | 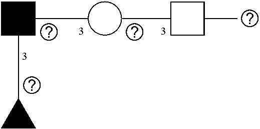 |
| C1Lac1 or C2G | 983.5146 | 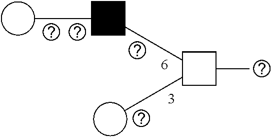 |
| | | 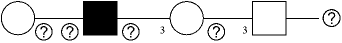 |
| C1F1S1 | 1069.5514 | 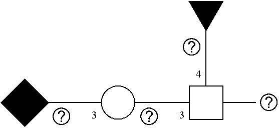 |
| | | 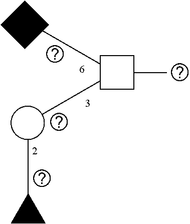 |
| (C1H1S1) or H2N1S1 | 1099.5619 | 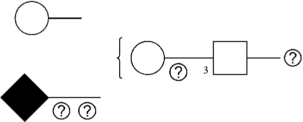 |
| | | 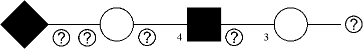 |
| C1N1S1 or C2S1 | 1140.5885 | 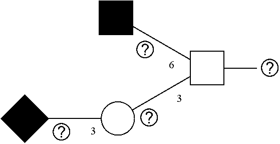 |
| | | 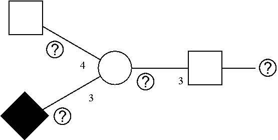 |
| | | 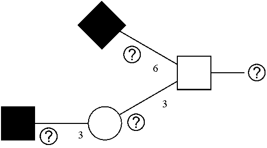 |
| C1Lac1F1 or C2GF1 | 1157.6038 | 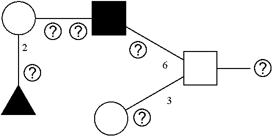 |
| | | 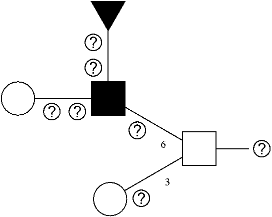 |
| | | 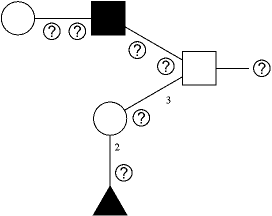 |
| | | 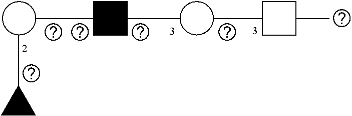 |
| | | 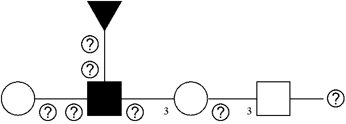 |
| C1S2 or N1H1S2 | 1256.6358 | 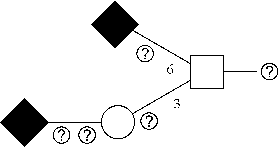 |
| | | 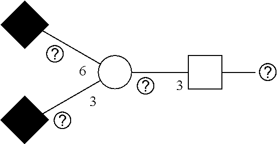 |
| | | 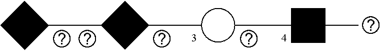 |
| C1N1F1S1 or C2F1S1 or C3H1F1S1 | 1314.6777 | 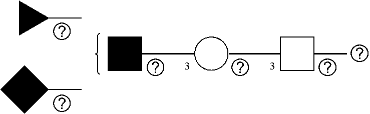 |
| | | 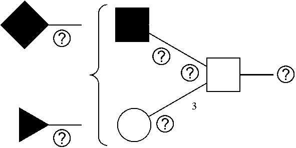 |
| | | 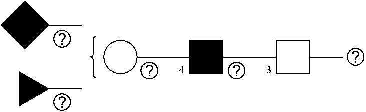 |
| C1Lac1S1 or C2GS1 | 1344.6882 | 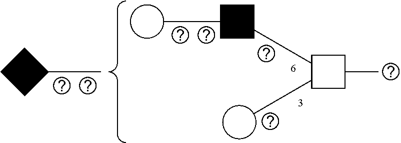 |
| | | 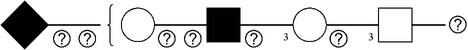 |
| C1Lac1F1S1 or C2GF1S1 | 1518.7775 | 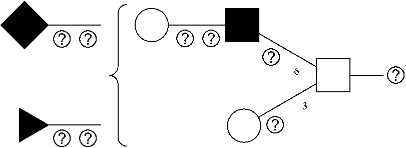 |
| | | 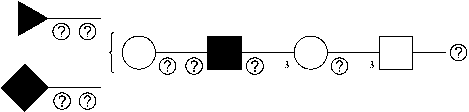 |
| C1S3 | 1617.8095 | 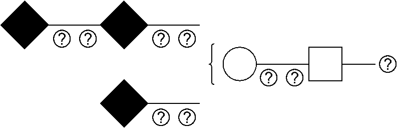 |
| | | 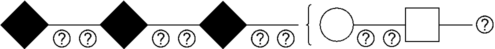 |
| C2GN2F1 | 1647.8564 | 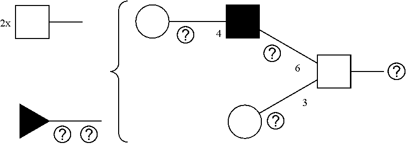 |
| C1Lac2N1 or C1N1Lac1S1 or C2GLac1N1 or (N);A2 | 1677.8670 | 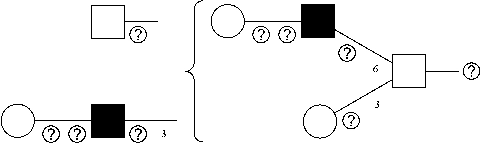 |
| | | 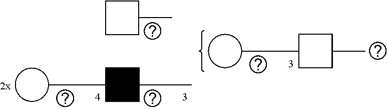 |
| | | 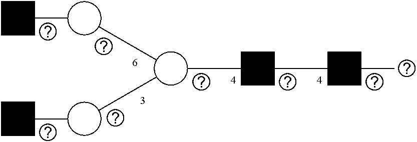 |
| C1Lac1S2 or C2GS2 | 1705.8619 | 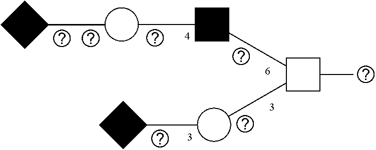 |
| | | 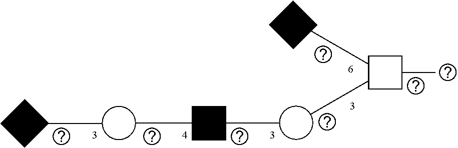 |
| C2GN1F1S1 or C4Ga2F1S1 | 1763.9038 | 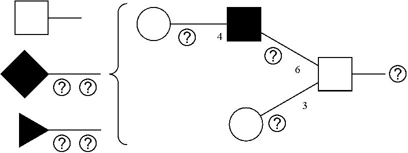 |
| | | 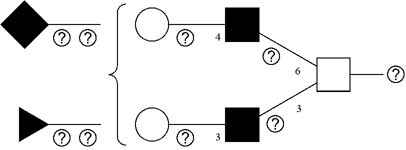 |
| C2GLac1S1 | 1793.9143 | 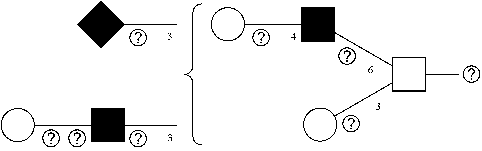 |
| C2GN1Lac1F1 or (N);FA2 | 1851.9562 | 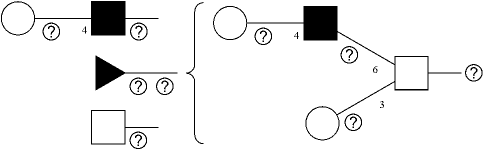 |
| | | 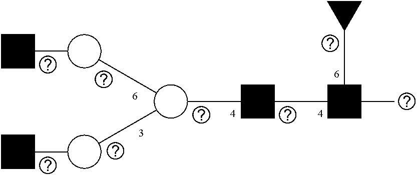 |
| C1Lac3 or C2GLac2 or (N);A2G1 | 1881.9668 | 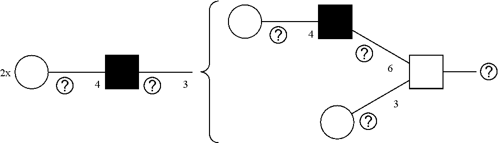 |
| | | 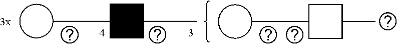 |
| | | 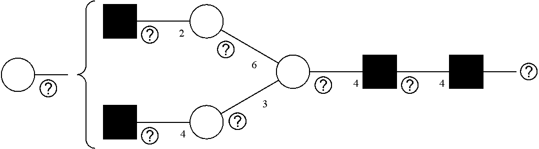 |
| C1Lac1N1S2 or C2GN1S2 or C3Ga1Lac1S2 or C4Ga2S2 | 1950.9882 | 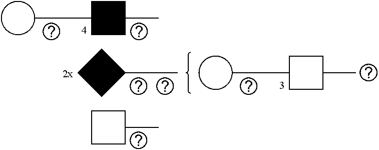 |
| | | 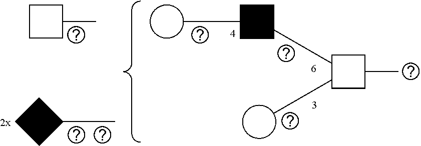 |
| | | 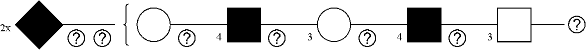 |
| | | 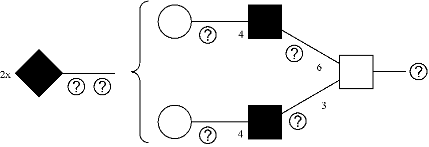 |
| C1Lac1S3 or C2GS3 | 2067.0356 | 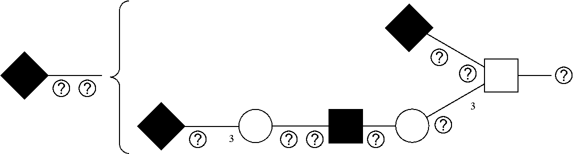 |
| | | 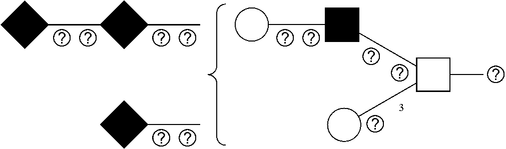 |
| C2Lac- 2N1F1 or C3Ga1Lac- 2N1F1 or (N);FA3 or (N);FA2B | 2097.0825 | 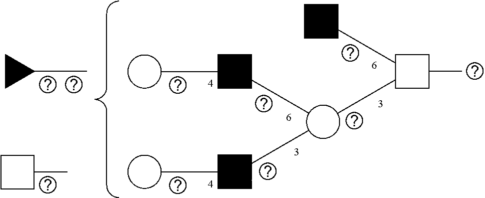 |
| | | 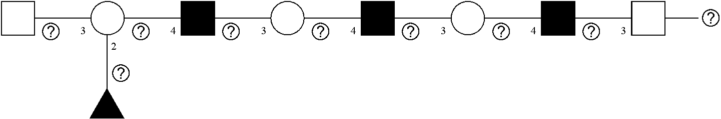 |
| | | 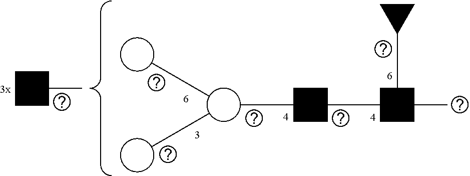 |
| C2GLac1S2 | 2155.0880 | 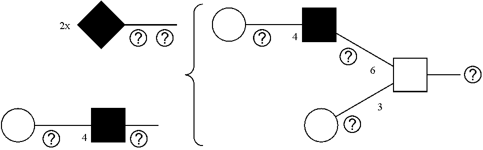 |
| C2Lac3F1 or (N);FA3G1 or (N);FA2BG1 | 2301.1823 | 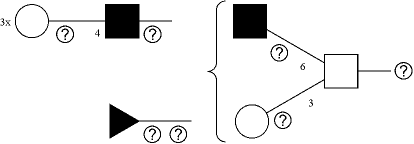 |
| | | 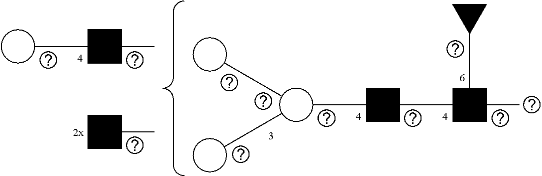 |

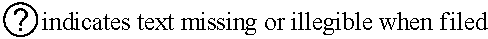

IL-15

As used herein, the terms “IL-15” and “interleukin-15” refer to a native IL-15 or an IL-15 derivative. As used herein, the terms “native IL-15” and “native interleukin-15” in the context of proteins or polypeptides refer to any naturally occurring and wild type mammalian interleukin-15 amino acid sequences, including immature or precursor and mature forms. Non-limiting examples of GeneBank Accession Nos. for the amino acid sequence of various species of native mammalian interleukin-15 include NP 000576 (human, immature form), CAA62616 (human, immature form), NP 001009207 (Felis catus, immature form), AAB94536 (Rattus norvegicus, immature form), AAB41697 (Rattus norvegicus, immature form), NP 032383 (Mus musculus, immature form), AAR19080 (canine), AAB60398 (Macaca mulatta, immature form), AAI00964 (human, immature form), AAH23698 (Mus musculus, immature form), and AAH18149 (human). The amino acid sequence of the immature/precursor form of native human IL-15, which comprises the long signal peptide (underlined) and the mature human native IL-15 (italicized), as provided in SEQ ID NO: 1 in Table 2. In some embodiments, native IL-15 is the immature or precursor form of a naturally occurring or wild type mammalian IL-15. In other embodiments, native IL-15 is the mature form of a naturally occurring or wild type mammalian IL-15. In a specific embodiment, native IL-15 is the precursor form of naturally occurring or wild type human IL-15. In another embodiment, native IL-15 is the mature form of naturally occurring or wild type human IL-15. In one embodiment, the native IL-15 protein/polypeptide is isolated or purified.

As used herein, the terms “native IL-15” and “native “interleukin-15” in the context of nucleic acids refer to any naturally occurring nucleic acid sequences or wild type nucleic acid sequences encoding mammalian interleukin-15, including the immature or precursor and mature forms. Non-limiting examples of GeneBank Accession Nos. for the nucleotide sequence of various species of native mammalian IL-15 include NM 000585 (human), NM 008357 (Mus musculus), and RNU69272 (Rattus norvegicus). The nucleotide sequence encoding the immature/precursor form of native human IL-15, which comprises the nucleotide sequence encoding the long signal peptide (underlined) and the nucleotide sequence encoding the mature human native IL-15 (italicized), as provided in SEQ ID NO: 2 in Table 2. In a specific embodiment, the nucleic acid is an isolated or purified nucleic acid. In some embodiments, nucleic acids encode the immature or precursor form of a naturally occurring or wild type mammalian IL-15. In other embodiments, nucleic acids encode the mature form of a naturally occurring or wild type mammalian IL-15. In a specific embodiment, nucleic acids encoding native IL-15 encode the precursor form of naturally occurring or wild type human IL-15. In another embodiment, nucleic acids encoding native IL-15 encode the mature form of naturally occurring or wild type human IL-15.

As used herein, the terms “IL-15 derivative” and “interleukin-15 derivative” in the context of proteins or polypeptides refer to: (a) a polypeptide that is at least 75%, 80%, 85%, 90%, 95%, 98% or 99% identical to a native mammalian IL-15 polypeptide; (b) a polypeptide encoded by a nucleic acid sequence that is at least 75%, 80%, 85%, 90%, 95%, 98% or 99% identical a nucleic acid sequence encoding a native mammalian IL-15 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-15 polypeptide; (d) a polypeptide encoded by nucleic acids can hybridize under high, moderate or typical stringency hybridization conditions to nucleic acids encoding a native mammalian IL-15 polypeptide; (e) a polypeptide encoded by a nucleic acid sequence that can hybridize under high, moderate or typical stringency hybridization conditions to a nucleic acid sequence encoding a fragment of a native mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; and/or (f) a fragment of a native mammalian IL-15 polypeptide. IL-15 derivatives also include a polypeptide that comprises the amino acid sequence of a naturally occurring or wild type mature form of a mammalian IL-15 polypeptide and a heterologous signal peptide amino acid sequence. In a specific embodiment, an IL-15 derivative is a derivative of a native human IL-15 polypeptide. In another embodiment, an IL-15 derivative is a derivative of an immature or precursor form of naturally occurring or wild type human IL-15 polypeptide. In another embodiment, an IL-15 derivative is a derivative of a mature form of naturally occurring or wild type human IL-15 polypeptide. In another embodiment, an IL-15 derivative is the IL-15N72D described in, e.g., Zhu et al., (2009), J. Immunol. 183: 3598 or U.S. Pat. No. 8,163,879. In another embodiment, an IL-15 derivative is one of the IL-15 variants described in U.S. Pat. No. 8,163,879. In one embodiment, an IL-15 derivative is isolated or purified.

In a preferred embodiment, IL-15 derivatives retain at least 75%, 80%, 85%, 90%, 95%, 98% or 99% of the function of native mammalian IL-15 polypeptide to bind IL-15Rα polypeptide, as measured by assays well known in the art, e.g., ELISA, BIAcore™, co-immunoprecipitation. In another preferred embodiment, IL-15 derivatives retain at least 75%, 80%, 85%, 90%, 95%, 98% or 99% of the function of native mammalian IL-15 polypeptide to induce IL-15-mediated signal transduction, as measured by assays well-known in the art, e.g., electromobility shift assays, ELISAs and other immunoassays. In a specific embodiment, IL-15 derivatives bind to IL-15Rα and/or IL-15Rβγ as assessed by, e.g., ligand/receptor binding assays well-known in the art. Percent identity can be determined using any method known to one of skill in the art and as described supra.

As used herein, the terms “IL-15 derivative” and “interleukin-15 derivative” in the context of nucleic acids refer to: (a) a nucleic acid sequence that is at least 75%, 80%, 85%, 90%, 95%, 98% or 99% identical to the naturally occurring or wild type nucleic acid sequence encoding a mammalian IL-15 polypeptide; (b) a nucleic acid sequence encoding a polypeptide that is at least 75%, 80%, 85%, 90%, 95%, 98% or 99% identical the amino acid sequence of a native mammalian IL-15 polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid base mutations (i.e., additions, deletions and/or substitutions) relative to the naturally occurring or wild type nucleic acid sequence encoding a mammalian IL-15 polypeptide; (d) a nucleic acid sequence that hybridizes under high, moderate or typical stringency hybridization conditions to a naturally occurring or wild type nucleic acid sequence encoding a mammalian IL-15 polypeptide; (e) a nucleic acid sequence that hybridizes under high, moderate or typical stringency hybridization conditions to a fragment of a naturally occurring or wild type nucleic acid sequence encoding a mammalian IL-15 polypeptide; and/or (f) a nucleic acid sequence encoding a fragment of a naturally occurring or wild type nucleic acid sequence encoding a mammalian IL-15 polypeptide. In a specific embodiment, an IL-15 derivative in the context of nucleic acids is a derivative of a naturally occurring or wild type nucleic acid sequence encoding a human IL-15 polypeptide. In another embodiment, an IL-15 derivative in the context of nucleic acids is a derivative of a naturally occurring or wild type nucleic acid sequence encoding an immature or precursor form of a human IL-15 polypeptide. In another embodiment, an IL-15 derivative in the context of nucleic acids is a derivative of a naturally occurring or wild type nucleic acid sequence encoding a mature form of a human IL-15 polypeptide. In another embodiment, an IL-15 derivative in the context of nucleic acids is the nucleic acid sequence encoding the IL-15N72D described in, e.g., Zhu et al., (2009; supra), or U.S. Pat. No. 8,163,879. In another embodiment, an IL-15 derivative in the context of nucleic acids is the nucleic acid sequence encoding one of the IL-15 variants described in U.S. Pat. No. 8,163,879.

IL-15 derivative nucleic acid sequences include codon-optimized nucleic acid sequences that encode native mammalian IL-15 polypeptide, including mature and immature forms of IL-15 polypeptide. In other embodiments, IL-15 derivative nucleic acids include nucleic acids that encode mammalian IL-15 RNA transcripts containing mutations that eliminate potential splice sites and instability elements (e.g., A/T or A/U rich elements) without affecting the amino acid sequence to increase the stability of the mammalian IL-15 RNA transcripts. In one embodiment, the IL-15 derivative nucleic acid sequences include the codon-optimized nucleic acid sequences described in WO2007/084342. In certain embodiments, the IL-15 derivative nucleic acid sequence is the codon-optimized sequence in SEQ ID NO: 4 in Table 2 (the amino acid sequence encoded by such a nucleic acid sequence is provided in SEQ ID NO: 5 in Table 2).

In a preferred embodiment, IL-15 derivative nucleic acid sequences encode proteins or polypeptides that retain at least 75%, 80%, 85%, 90%, 95%, 98% or 99% of the function of a native mammalian IL-15 polypeptide to bind IL-15Rα, as measured by assays well known in the art, e.g., ELISA, BIAcore™, co-immunoprecipitation. In another preferred embodiment, IL-15 derivative nucleic acid sequences encode proteins or polypeptides that retain at least 75%, 80%, 85%, 90%, 95%, 98% or 99% of the function of a native mammalian IL-15 polypeptide to induce IL-15-mediated signal transduction, as measured by assays well-known in the art, e.g., electromobility shift assays, ELISAs and other immunoassays. In a specific embodiment, IL-15 derivative nucleic acid sequences encode proteins or polypeptides that bind to IL-15Rα and/or IL-15Rβγ as assessed by, e.g., ligand/receptor assays well-known in the art.

IL-15Rα

As used herein, the terms “IL-15Rα” and “interleukin-15 receptor alpha” refer to a native IL-15Rα, an IL-15Rα derivative, or a native IL-15Rα and an IL-15Rα derivative. As used herein, the terms “native IL-15Rα” and “native interleukin-15 receptor alpha” in the context of proteins or polypeptides refer to any naturally occurring and wild type mammalian interleukin-15 receptor alpha (“IL-15Rα”) amino acid sequence, including immature or precursor and mature forms and naturally occurring isoforms. Non-limiting examples of GeneBank Accession Nos. for the amino acid sequence of various native mammalian IL-15Rα include NP 002180 (human), ABK41438 (Macaca mulatta), NP 032384 (Mus musculus), Q60819 (Mus musculus), CAI41082 (human). The amino acid sequence of the immature form of the native full length human IL-15Rα, which comprises the signal peptide (underlined) and the mature human native IL-15Rα (italicized), as provided in SEQ ID NO: 6 in Table 2. The amino acid sequence of the immature form of the native soluble human IL-15Rα, which comprises the signal peptide (underlined) and the mature human native soluble IL-15Rα (italicized), as provided in SEQ ID NO:7 in Table 2. In some embodiments, native IL-15Rα is the immature form of a naturally occurring or wild type mammalian IL-15Rα polypeptide. In other embodiments, native IL-15Rα is the mature form of a naturally occurring or wild type mammalian IL-15Rα polypeptide. In certain embodiments, native IL-15Rα is the naturally occurring or wild type soluble form of mammalian IL-15Rα polypeptide. In other embodiments, native IL-15Rα is the full-length form of a naturally occurring or wild type mammalian IL-15Rα polypeptide. In a specific embodiment, native IL-15Rα is the immature form of a naturally occurring or wild type human IL-15Rα polypeptide. In another embodiment, native IL-15Rα is the mature form of a naturally occurring or wild type human IL-15Rα polypeptide. In certain embodiments, native IL-15Rα is the naturally occurring or wild type soluble form of human IL-15Rα polypeptide. In other embodiments, native IL-15Rα is the full-length form of a naturally occurring or wild type human IL-15Rα polypeptide. In one embodiment, a native IL-15Rα protein or polypeptide is isolated or purified.

As used herein, the terms “native IL-15Rα” and “native interleukin-15 receptor alpha” in the context of nucleic acids refer to any naturally occurring nucleic acid sequences or wild type nucleic acid sequences encoding mammalian interleukin-15 receptor alpha, including the immature or precursor and mature forms. Non-limiting examples of GeneBank Accession Nos. for the nucleotide sequence of various species of native mammalian IL-15Rα include NM_002189 (human), EF033114 (Macaca mulatta), and NM 008358 (Mus musculus). The nucleotide sequence encoding the immature form of native human IL-15Rα, which comprises the nucleotide sequence encoding the signal peptide (underlined) and the nucleotide sequence encoding the mature human native IL-15Rα (italicized), as provided in SEQ ID NO: 8 in Table 2. The nucleotide sequence encoding the immature form of native soluble human IL-15Rα protein or polypeptide, which comprises the nucleotide sequence encoding the signal peptide (underlined) and the nucleotide sequence encoding the mature human soluble native IL-15Rα (italicized), as provided in SEQ ID NO: 9 in Table 2). In a specific embodiment, the nucleic acid is an isolated or purified nucleic acid. In some embodiments, naturally occurring nucleic acids encode the immature form of a naturally occurring or wild type mammalian IL-15Rα polypeptide. In other embodiments, naturally occurring nucleic acids encode the mature form of a naturally occurring or wild type mammalian IL-15Rα polypeptide. In certain embodiments, naturally occurring nucleic acids encode the soluble form of a naturally occurring or wild type mammalian IL-15Rα polypeptide. In other embodiments, naturally occurring nucleic acids encode the full-length form of a naturally occurring or wild type mammalian IL-15Rα polypeptide. In a specific embodiment, naturally occurring nucleic acids encode the precursor form of naturally occurring or wild type human IL-15 polypeptide. In another embodiment, naturally occurring nucleic acids encode the mature of naturally occurring or wild type human IL-15 polypeptide. In certain embodiments, naturally occurring nucleic acids encode the soluble form of a naturally occurring or wild type human IL-15Rα polypeptide. In other embodiments, naturally occurring nucleic acids encode the full-length form of a naturally occurring or wild type human IL-15Rα polypeptide.

As used herein, the terms “IL-15Rα derivative” and “interleukin-15 receptor alpha derivative” in the context of a protein or polypeptide refer to: (a) a polypeptide that is at least 75%, 80%, 85%, 90%, 95%, 98% or 99% identical to a native mammalian IL-15 polypeptide; (b) a polypeptide encoded by a nucleic acid sequence that is at least 75%, 80%, 85%, 90%, 95%, 98% or 99% identical a nucleic acid sequence encoding a native mammalian IL-15Rα polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-15Rα polypeptide; (d) a polypeptide encoded by a nucleic acid sequence that can hybridize under high, moderate or typical stringency hybridization conditions to a nucleic acid sequence encoding a native mammalian IL-15Rα polypeptide; (e) a polypeptide encoded by a nucleic acid sequence that can hybridize under high, moderate or typical stringency hybridization conditions to nucleic acid sequences encoding a fragment of a native mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; (f) a fragment of a native mammalian IL-15Rα polypeptide; and/or (g) a specific IL-15Rα derivative described herein. IL-15Rα derivatives also include a polypeptide that comprises the amino acid sequence of a naturally occurring or wild type mature form of mammalian IL-15Rα polypeptide and a heterologous signal peptide amino acid sequence. In a specific embodiment, an IL-15Rα derivative is a derivative of a native human IL-15Rα polypeptide. In another embodiment, an IL-15Rα derivative is a derivative of an immature form of naturally occurring or wild type human IL-15 polypeptide. In another embodiment, an IL-15Rα derivative is a derivative of a mature form of naturally occurring or wild type human IL-15 polypeptide. In one embodiment, an IL-15Rα derivative is a soluble form of a native mammalian IL-15Rα polypeptide. In other words, in certain embodiments, an IL-15Rα derivative includes soluble forms of native mammalian IL-15Rα, wherein those soluble forms are not naturally occurring. Other examples of IL-15Rα derivatives include the truncated, soluble forms of native human IL-15Rα described herein. In a specific embodiment, an IL-15Rα derivative is purified or isolated.

In a preferred embodiment, IL-15Rα derivatives retain at least 75%, 80%, 85%, 90%, 95%, 98% or 99% of the function of a native mammalian IL-15Rα polypeptide to bind an IL-15 polypeptide, as measured by assays well known in the art, e.g., ELISA, BIAcore™, co-immunoprecipitation. In another preferred embodiment, IL-15Rα derivatives retain at least 75%, 80%, 85%, 90%, 95%, 98% or 99% of the function of a native mammalian IL-15Rα polypeptide to induce IL-15-mediated signal transduction, as measured by assays well-known in the art, e.g., electromobility shift assays, ELISAs and other immunoassays. In a specific embodiment, IL-15Rα derivatives bind to IL-15 as assessed by methods well-known in the art, such as, e.g., ELISAs.

As used herein, the terms “IL-15Rα derivative” and “interleukin-15 receptor alpha derivative” in the context of nucleic acids refer to: (a) a nucleic acid sequence that is at least 75%, 80%, 85%, 90%, 95%, 98% or 99% identical to the naturally occurring or wild type nucleic acid sequence encoding a mammalian IL-15Rα polypeptide; (b) a nucleic acid sequence encoding a polypeptide that is at least 75%, 80%, 85%, 90%, 95%, 98% or 99% identical the amino acid sequence of a native mammalian IL-15Rα polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid mutations (i.e., additions, deletions and/or substitutions) relative to the naturally occurring or wild type nucleic acid sequence encoding a mammalian IL-15Rα polypeptide; (d) a nucleic acid sequence that hybridizes under high, moderate or typical stringency hybridization conditions to a naturally occurring or wild type nucleic acid sequence encoding a mammalian IL-15Rα polypeptide; (e) a nucleic acid sequence that hybridizes under high, moderate or typical stringency hybridization conditions to a fragment of a naturally occurring or wild type nucleic acid sequence encoding a mammalian IL-15Rα polypeptide; (f) a nucleic acid sequence encoding a fragment of a naturally occurring or wild type nucleic acid sequence encoding a mammalian IL-15Rα polypeptide; and/or (g) a nucleic acid sequence encoding a specific IL-15Rα derivative described herein. In a specific embodiment, an IL-15Rα derivative in the context of nucleic acids is a derivative of a naturally occurring or wild type nucleic acid sequence encoding a human IL-15Rα polypeptide. In another embodiment, an IL-15Rα derivative in the context of nucleic acids is a derivative of a naturally occurring or wild type nucleic acid sequence encoding an immature form of a human IL-15Rα polypeptide. In another embodiment, an IL-15Rα derivative in the context of nucleic acids is a derivative of a naturally occurring or wild type nucleic acid sequence encoding a mature form of a human IL-15Rα polypeptide. In one embodiment, an IL-15Rα derivative in the context of nucleic acids refers to a nucleic acid sequence encoding a derivative of mammalian IL-15Rα polypeptide that is soluble. In certain embodiments, an IL-15Rα derivative in context of nucleic acids refers to a nucleic acid sequence encoding a soluble form of native mammalian IL-15Rα, wherein the soluble form is not naturally occurring. In some embodiments, an IL-15Rα derivative in the context of nucleic acids refers to a nucleic acid sequence encoding a derivative of human IL-15Rα, wherein the derivative of the human IL-15Rα is a soluble form of IL-15Rα that is not naturally occurring. In specific embodiments, an IL-15Rα derivative nucleic acid sequence is isolated or purified.

IL-15Rα derivative nucleic acid sequences include codon-optimized nucleic acid sequences that encode native IL-15Rα polypeptide, including mature and immature forms of IL-15Rα polypeptide. In other embodiments, IL-15Rα derivative nucleic acids include nucleic acids that encode IL-15Rα RNA transcripts containing mutations that eliminate potential splice sites and instability elements (e.g., A/T or A/U rich elements) without affecting the amino acid sequence to increase the stability of the IL-15Rα RNA transcripts. In certain embodiments, the IL-15Rα derivative nucleic acid sequence is the codon-optimized sequence in SEQ ID NO: 11, 13 in Table 2 (the amino acid sequences encoded by such a nucleic acid sequences are provided in SEQ ID NO: 12, 14 in Table 2, respectively).

In specific embodiments, IL-15Rα derivative nucleic acid sequences encode proteins or polypeptides that retain at least 50%, 55%, 60%, 65%, 70%, 75%, 80%, 85%, 90%, 95%, 98% or 99% of the function of a native mammalian IL-15Rα polypeptide to bind IL-15, as measured by assays well known in the art, e.g., ELISA, BIAcore™, co-immunoprecipitation. In another preferred embodiment, IL-15Rα derivative nucleic acid sequences encode proteins or polypeptides that retain at least 50%, 55%, 60%, 65%, 70%, 75%, 80%, 85%, 90%, 95%, 98% or 99% of the function of a native mammalian IL-15Rα to induce IL-15-mediated signal transduction, as measured by assays well-known in the art, e.g., electromobility shift assays, ELISAs and other immunoassays. In a specific embodiment, IL-15Rα derivative nucleic acid sequences encode proteins or polypeptides that bind to IL-15 as assessed by methods well-known in the art, such as, e.g., ELISAs.

Described herein is the naturally occurring or wild type soluble form of human IL-15Rα. Also described herein are specific IL-15Rα derivatives that are truncated, soluble forms of human IL-15Rα. These specific IL-15Rα derivatives and the naturally occurring or wild type soluble form of human IL-15Rα are based, in part, on the identification of the proteolytic cleavage site of human IL-15Rα. Further described herein are soluble forms of IL-15Rα that are characterized based upon glycosylation of the IL-15Rα.

The proteolytic cleavage of human IL-15Rα takes place between the residues (i.e., Gly170 and His171) which are in shown in bold and underlined in the provided amino acid sequence of the immature form of the native full length human IL-15Rα:

(SEQ ID NO: 6 in Table 2)
MAPRRARGCR TLGLPALLLL LLLRPPATRG ITCPPPMSVE
HADIWVKSYS LYSRERYICN SGFKRKAGTS SLTECVLNKA
TNVAHWTTPS LKCIRDPALV HQRPAPPSTV TTAGVTPQPE
SLSPSGKEPA ASSPSSNNTA ATTAAIVPGS QLMPSKSPST
GTTEISSHES SHGTPSQTTA KNWELTASAS HQPPGVYPQG
HSDTTVAIST STVLLCGLSA VSLLACYLKS RQTPPLASVE
MEAMEALPVT WGTSSRDEDL ENCSHHL.

Accordingly, in one aspect, provided herein is a soluble form of human IL-15Rα (e.g., a purified soluble form of human IL-15Rα), wherein the amino acid sequence of the soluble form of human IL-15Rα terminates at the site of the proteolytic cleavage of the native membrane-bound human IL-15Rα. In particular, provided herein is a soluble form of human IL-15Rα (e.g., a purified soluble form of human IL-15Rα), wherein the amino acid sequence of the soluble form of human IL-15Rα terminates with PQG (SEQ ID NO: 20 in Table 2), wherein G is Gly170. In a particular embodiment, provided herein is a soluble form of human IL-15Rα (e.g., a purified soluble form of human IL-15Rα) which has the amino acid sequence shown in SEQ ID NO: 7 in Table 2. In some embodiments, provided herein is an IL-15Rα derivative (e.g., a purified and/or soluble form of IL-15Rα derivative), which is a polypeptide that: (i) is at least 75%, 80%, 85%, 90%, 95%, 98% or 99% identical to SEQ ID NO: 7 in Table 2; and (ii) terminates with the amino acid sequence PQG (SEQ ID NO: 20 in Table 2). In other particular embodiments, provided herein is a soluble form of human IL-15Rα (e.g., a purified soluble form of human IL-15Rα) which has the amino acid sequence of SEQ ID NO: 10 in Table 2). In some embodiments, provided herein is an IL-15Rα derivative (e.g., a purified and/or soluble form of an IL-15Rα derivative), which is a polypeptide that is at least 75%, 80%, 85%, 90%, 95%, 98% or 99% identical to SEQ ID NO: 10 in Table 2, and, optionally, wherein the amino acid sequence of the soluble form of the IL-15Rα derivative terminates with PQG (SEQ ID NO: 20 in Table 2).

In some embodiments, provided herein is an IL-15Rα derivative of naturally occurring or wild type human IL-15Rα, wherein the IL-15Rα derivative is soluble and: (a) the last amino acids at the C-terminal end of the IL-15Rα derivative consist of amino acid residues PQGHSDTT (SEQ ID NO: 15 in Table 2); (b) the last amino acids at the C-terminal end of the IL-15Rα derivative consist of amino acid residues PQGHSDT (SEQ ID NO: 16 in Table 2); (c) the last amino acids at the C-terminal end of the IL-15Rα derivative consist of amino acid residues PQGHSD (SEQ ID NO: 17 in Table 2); (d) the last amino acids at the C-terminal end of the IL-15Rα derivative consist of amino acid residues PQGHS (SEQ ID NO: 18 in Table 2); or (e) the last amino acids at the C-terminal end of the IL-15Rα derivative consist of amino acid residues PQGH (SEQ ID NO: 19 in Table 2). In certain embodiments, the amino acid sequences of these IL-15Rα derivatives are at least 75%, at least 85%, at least 90%, at least 95%, at least 96%, at least 97%, at least 98%, or at least 99% identical to the amino acid sequence of SEQ ID NO: 21 in Table 2. In some embodiments, these IL-15Rα derivatives are purified.

In another aspect, provided herein are glycosylated forms of IL-15Rα (e.g., purified glycosylated forms of IL-15Rα), wherein the glycosylation of the IL-15Rα accounts for at least 20%, at least 25%, at least 30%, at least 35%, at least 40%, at least 45%, at least 50%, or 20% to 25%, 20% to 30%, 25% to 30%, 25% to 35%, 30% to 35%, 30% to 40%, 35% to 40%, 35% to 45%, 40% to 50%, 45% to 50%, 20% to 40%, or 25% to 50% of the mass (molecular weight) of the IL-15Rα as assessed by techniques known to one of skill in the art. The percentage of the mass (molecular weight) of IL-15Rα (e.g., purified IL-15Rα) that glycosylation of IL-15Rα accounts for can be determined using, for example and without limitation, gel electrophoresis and quantitative densitometry of the gels, and comparison of the average mass (molecular weight) of a glycosylated form of IL-15Rα (e.g., a purified glycosylated form of IL-15Rα) to the non-glycosylated form of IL-15Rα (e.g., a purified non-glycosylated form of IL-15Rα). In one embodiment, the average mass (molecular weight) of IL-15Rα (e.g., purified IL-15Rα) can be determined using MALDI-TOF MS spectrum on Voyager De-Pro equipped with CovalX HM-1 high mass detector using sinapic acid as matrix, and the mass of a glycosylated form of IL-15Rα (e.g., purified glycosylated form of IL-15Rα) can be compared to the mass of the non-glycosylated form of IL-15Rα (e.g., purified non-glycosylated form of IL-15Rα) to determine the percentage of the mass that glycosylation accounts for.

In another aspect, provided herein are glycosylated forms of IL-15Rα, wherein the IL-15Rα is glycosylated (N- or O-glycosylated) at certain amino acid residues. In certain embodiments, provided herein is a human IL-15Rα which is glycosylated at one, two, three, four, five, six, seven, or all, of the following glycosylation sites: (i) O-glycosylation on threonine at position 5 of the amino acid sequence NWELTASASHQPPGVYPQG (SEQ ID NO: 22 in Table 2) in the IL-15Rα; (ii) O-glycosylation on serine at position 7 of the amino acid sequence NWELTASASHQPPGVYPQG (SEQ ID NO: 22 in Table 2) in the IL-15Rα; (iii) N-glycosylation on serine at position 8 of the amino acid sequence ITCPPPMSVEHADIWVK (SEQ ID NO: 22 in Table 2) in the IL-15Rα, or serine at position 8 of the amino acid sequence ITCPPPMSVEHADIWVKSYSLYSRERYICNS (SEQ ID NO: 23 in Table 2) in the IL-15Rα; (iv) N-glycosylation on Ser 18 of amino acid sequence ITCPPPMSVEHADIWVKSYSLYSRERYICNS (SEQ ID NO: 24 in Table 2) in the IL-15Rα; (v) N-glycosylation on serine at position 20 of the amino acid sequence ITCPPPMSVEHADIWVKSYSLYSRERYICNS (SEQ ID NO: 24 in Table 2) in the IL-15Rα; (vi) N-glycosylation on serine at position 23 of the amino acid sequence ITCPPPMSVEHADIWVKSYSLYSRERYICNS (SEQ ID NO: 24 in Table 2) in the IL-15Rα; and/or (vii) N-glycosylated on serine at position 31 of the amino acid sequence ITCPPPMSVEHADIWVKSYSLYSRERYICNS (SEQ ID NO: 24 in Table 2) in the IL-15Rα. In specific embodiments, the glycosylated IL-15Rα is a native human IL-15Rα. In other specific embodiments, the glycosylated IL-15Rα is an IL-15Rα derivative of naturally occurring or wild type human IL-15Rα. In some embodiments, the glycosylated IL-15Rα is a native soluble human IL-15Rα, such as SEQ ID NO: 7 or 10 in Table 2. In other embodiments, the glycosylated IL-15Rα is an IL-15Rα derivative that is a soluble form of human IL-15Rα. In certain embodiments, the glycosylated IL-15Rα is purified or isolated.

IL-15/IL-15Rα Complex

As used herein, the term “IL-15/IL-15Rα complex” refers to a complex comprising IL-15 and IL-15Rα covalently or noncovalently bound to each other. In a preferred embodiment, the IL-15Rα has a relatively high affinity for IL-15, e.g., KD of 10 to 50 pM as measured by a technique known in the art, e.g., KinEx A assay, plasma surface resonance (e.g., BIAcore™ assay). In another preferred embodiment, the IL-15/IL-15Rα complex induces IL-15-mediated signal transduction, as measured by assays well-known in the art, e.g., electromobility shift assays, ELISAs and other immunoassays. In some embodiments, the IL-15/IL-15Rα complex retains the ability to specifically bind to the βγ chain. In a specific embodiment, the IL-15/IL-15Rα complex is isolated from a cell.

Provided herein are complexes that bind to the βγ subunits of the IL-15 receptor, induce IL-15 signal transduction (e.g., Jak/Stat signal transduction) and enhance IL-15-mediated immune function, wherein the complexes comprise IL-15 covalently or noncovalently bound to interleukin-15 receptor alpha (“IL-15Rα”) (a “IL-15/IL-15Rα complex”). The IL-15/IL-15Rα complex is able to bind to the βγ receptor complex.

The IL-15/IL-15Rα complexes may be composed of native IL-15 or an IL-15 derivative and native IL-15Rα or an IL-15Rα derivative. In certain embodiments, an IL-15/IL-15Rα complex comprises native IL-15 or an IL-15 derivative and an IL-15Rα described above. In a specific embodiment, an IL-15/IL-15Rα complex comprises native IL-15 or an IL-15 derivative and IL-15Rα with the amino acid sequence of SEQ ID NO: 10 in Table 2. In another embodiment, an IL-15/IL-15Rα complex comprises native IL-15 or an IL-15 derivative and a glycosylated form of IL-15Rα described supra.

In a specific embodiment, an IL-15/IL-15Rα complex comprises native IL-15 or an IL-15Rα derivative and native soluble IL-15Rα (e.g., native soluble human IL-15Rα). In another specific embodiment, an IL-15/IL-15Rα complex is composed of an IL-15 derivative and an IL-15Rα derivative. In another embodiment, an IL-15/IL-15Rα complex is composed of native IL-15 and an IL-15Rα derivative. In one embodiment, the IL-15Rα derivative is a soluble form of IL-15Rα. Specific examples of soluble forms of IL-15Rα are described above. In a specific embodiment, the soluble form of IL-15Rα lacks the transmembrane domain of native IL-15Rα, and optionally, the intracellular domain of native IL-15Rα. In another embodiment, the IL-15Rα derivative is the extracellular domain of native IL-15Rα or a fragment thereof. In certain embodiments, the IL-15Rα derivative is a fragment of the extracellular domain comprising the sushi domain or exon 2 of native IL-15Rα. In some embodiments, the IL-15Rα derivative comprises a fragment of the extracellular domain comprising the sushi domain or exon 2 of native IL-15Rα and at least one amino acid that is encoded by exon 3. In certain embodiments, the IL-15Rα derivative comprises a fragment of the extracellular domain comprising the sushi domain or exon 2 of native IL-15Rα and an IL-15Rα hinge region or a fragment thereof. In certain embodiments, the IL-15Rα comprises the amino acid sequence of SEQ ID NO: 10 in Table 2.

In another embodiment, the IL-15Rα derivative comprises a mutation in the extracellular domain cleavage site that inhibits cleavage by an endogenous protease that cleaves native IL-15Rα. In a specific embodiment, the extracellular domain cleavage site of IL-15Rα is replaced with a cleavage site that is recognized and cleaved by a heterologous known protease. Non-limiting examples of such heterologous protease cleavage sites include Arg-X-X-Arg (SEQ ID NO: 25 in Table 2), which is recognized and cleaved by furin protease; and A-B-Pro-Arg-X-Y (SEQ ID NO: 26 in Table 2) (A and B are hydrophobic amino acids and X and Y are non-acidic amino acids) and Gly-Arg-Gly, which are recognized and cleaved by thrombin protease.

In another embodiment, the IL-15 is encoded by a nucleic acid sequence optimized to enhance expression of IL-15, e.g., using methods as described in WO 2007/084342 and WO 2010/020047; and U.S. Pat. Nos. 5,965,726; 6,174,666; 6,291,664; 6,414,132; and 6,794,498.

In certain embodiments, provided herein is an IL-15/IL-15Rα complex comprising human IL-15Rα which is glycosylated at one, two, three, four, five, six, seven, or all, of the glycosylation sites as described supra and with reference to SEQ ID NOs: 22, 23 and 24 in Table 2. In specific embodiments, the glycosylated IL-15Rα is a native human IL-15Rα. In other specific embodiments, the glycosylated IL-15Rα is an IL-15Rα derivative of naturally occurring or wild type human IL-15Rα. In some embodiments, the glycosylated IL-15Rα is a native soluble human IL-15Rα, such as SEQ ID NO: 7 or 10 in Table 2. In other embodiments, the glycosylated IL-15Rα is an IL-15Rα derivative that is a soluble form of human IL-15Rα. In certain embodiments, the IL-15/IL-15Rα complex is purified or isolated.

In addition to IL-15 and IL-15Rα, the IL-15/IL-15Rα complexes may comprise a heterologous molecule. In some embodiments, the heterologous molecule increases protein stability. Non-limiting examples of such molecules include polyethylene glycol (PEG), Fc domain of an IgG immunoglobulin or a fragment thereof, or albumin that increase the half-life of IL-15 or IL-15Rα in vivo. In certain embodiments, IL-15Rα is conjugated/fused to the Fc domain of an immunoglobulin (e.g., an IgG1) or a fragment thereof. In a specific embodiment, the IL-15RαFc fusion protein comprises the amino acid sequence of SEQ ID NO: 27 or 28 in Table 2. In another embodiment, the IL-15RαFc fusion protein is the IL-15Rα/Fc fusion protein described in Han et al., (2011), Cytokine 56: 804-810, U.S. Pat. Nos. 8,507,222 or 8,124,084. In those IL-15/IL-15Rα complexes comprising a heterologous molecule, the heterologous molecule may be conjugated to IL-15 and/or IL-15Rα. In one embodiment, the heterologous molecule is conjugated to IL-15Rα. In another embodiment, the heterologous molecule is conjugated to IL-15.

The components of an IL-15/IL-15Rα complex may be directly fused, using either non-covalent bonds or covalent bonds (e.g., by combining amino acid sequences via peptide bonds), and/or may be combined using one or more linkers. Linkers suitable for preparing the IL-15/IL-15Rα complexes comprise peptides, alkyl groups, chemically substituted alkyl groups, polymers, or any other covalently-bonded or non-covalently bonded chemical substance capable of binding together two or more components. Polymer linkers comprise any polymers known in the art, including polyethylene glycol (PEG). In some embodiments, the linker is a peptide that is 1, 2, 3, 4, 5, 6, 7, 8, 9, 10, 11, 12, 13, 14, 15, 16, 17, 18, 19, 20 or more amino acids long. In a specific embodiment, the linker is long enough to preserve the ability of IL-15 to bind to the IL-15Rα. In other embodiments, the linker is long enough to preserve the ability of the IL-15/IL-15Rα complex to bind to the βγ receptor complex and to act as an agonist to mediate IL-15 signal transduction.

In particular embodiments, IL-15/IL-15Rα complexes are pre-coupled prior to use in the methods described herein (e.g., prior to contacting cells with the IL-15/IL-15Rα complexes or prior to administering the IL-15/IL-15Rα complexes to a subject). In other embodiments, the IL-15/IL-15Rα complexes are not pre-coupled prior to use in the methods described herein.

In a specific embodiment, an IL-15/IL-15Rα complex enhances or induces immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the IL-15/IL-15Rα complex using assays well known in the art, e.g., ELISPOT, ELISA, and cell proliferation assays. In a specific embodiment, the immune function is cytokine release (e.g., interferon-gamma, IL-2, IL-5, IL-10, IL-12, or transforming growth factor (TGF)-beta). In one embodiment, the IL-15 mediated immune function is NK cell proliferation, which can be assayed, e.g., by flow cytometry to detect the number of cells expressing markers of NK cells (e.g., CD56). In another embodiment, the IL-15 mediated immune function is antibody production, which can be assayed, e.g., by ELISA. In some embodiments, the IL-15 mediated immune function is effector function, which can be assayed, e.g., by a cytotoxicity assay or other assays well known in the art.

In specific embodiments, examples of immune function enhanced by the IL-15/IL-15Rα complex include the proliferation/expansion of lymphocytes (e.g., increase in the number of lymphocytes), inhibition of apoptosis of lymphocytes, activation of dendritic cells (or antigen presenting cells), and antigen presentation. In particular embodiments, an immune function enhanced by the IL-15/IL-15Rα complex is proliferation/expansion in the number of or activation of CD4+ T cells (e.g., Th1 and Th2 helper T cells), CD8+ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122+ T cells, or natural killer cells (NK cells). In one embodiment, the IL-15/IL-15Rα complex enhances the proliferation/expansion or number of lymphocyte progenitors. In some embodiments, a IL-15/IL-15Rα complex increases the number of CD4+ T cells (e.g., Th1 and Th2 helper T cells), CD8+ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122+ T cells, or natural killer cells (NK cells) by approximately 1 fold, 2 fold, 3 fold, 4 fold, 5 fold, 6 fold, 7 fold, 8 fold, 9 fold, 10 fold, 20 fold, or more relative a negative control (e.g., number of the respective cells not treated, cultured, or contacted with an IL-15/IL-15Rα complex).

In a specific embodiment, the IL-15/IL-15Rα complex increases the expression of IL-2 on whole blood activated by Staphylococcal enterotoxin B (SEB). For example, the IL-15/IL-15Rα complex increases the expression of IL-2 by at least about 2, 3, 4, or 5-fold, compared to the expression of IL-2 when SEB alone is used.

Producting IL-15/IL-15Rα Complex

The nucleic acids encoding IL-15 and/or IL-15Rα can be inserted into nucleic acid constructs for expression in mammalian cells, bacteria, yeast, and viruses. IL-15 and IL-15Rα can be recombinantly expressed from the same nucleic acid construct (e.g., using a bicistronic nucleic acid construct) or from different nucleic acid constructs (e.g., using monocistronic nucleic acid constructs). In one embodiment, IL-15 and IL-15Rα can be recombinantly expressed from a single nucleic acid construct comprising a single open reading frame (ORF) of IL-15 and IL-15Rα.

The nucleic acid constructs may comprise one or more transcriptional regulatory element(s) operably linked to the coding sequence of IL-15 and/or IL-15Rα. The transcriptional regulatory elements are typically 5′ to the coding sequence and direct the transcription of the nucleic acids encoding IL-15 and/or IL-15Rα. In some embodiments, one or more of the transcriptional regulatory elements that are found in nature to regulate the transcription of the native IL-15 and/or native IL-15Rα gene are used to control transcription. In other embodiments, one or more transcriptional regulatory elements that are heterologous to the native IL-15 and/or native IL-15Rα gene are used to control transcription. Any transcriptional regulatory element(s) known to one of skill in the art may be used. Non-limiting examples of the types of transcriptional regulatory element(s) include a constitutive promoter, a tissue-specific promoter, and an inducible promoter. In a specific embodiment, transcription is controlled, at least in part, by a mammalian (in some embodiments, human) transcriptional regulatory element(s). In a specific embodiment, transcription is controlled, at least in part, by a strong promoter, e.g., CMV. In other aspects, an inducible promoter can be used.

The nucleic acid constructs also may comprise one or more post-transcriptional regulatory element(s) operably linked to the coding sequence of IL-15 and/or IL-15Rα. The post-transcriptional regulatory elements can be 5′ and/or 3′ to the coding sequence and direct the post-transcriptional regulation of the translation of RNA transcripts encoding IL-15 and/or IL-15Rα.

In another aspect, the nucleic acid construct can be a gene targeting vector that replaces a gene's existing regulatory region with a regulatory sequence isolated from a different gene or a novel regulatory sequence as described, e.g., in International Publication Nos. WO1994/12650 and WO2001/68882. In certain embodiments, a host cell can be engineered to increase production of endogenous IL-15 and/or IL-15Rα by, e.g., altering the regulatory region of the endogenous IL-15 and/or IL-15Rα genes.

The nucleic acid construct chosen will depend upon a variety of factors, including, without limitation, the strength of the transcriptional regulatory elements and the host cell to be used to express IL-15 and/or IL-15Rα. The nucleic acid constructs can be a plasmid, phagemid, cosmid, viral vector, phage, artificial chromosome, and the like. In one aspect, the vectors can be episomal or non-homologously integrating vectors, which can be introduced into the appropriate host cells by any suitable means (transformation, transfection, conjugation, protoplast fusion, electroporation, calcium phosphate-precipitation, direct microinjection, etc.) to transform them.

The nucleic acid constructs can be a plasmid or a stable integration vector for transient or stable expression of IL-15 and/or IL-15Rα in host cells. For stable expression, the vector can mediate chromosomal integration at a target site or a random chromosomal site. Non-limiting examples of host cell-vector systems that may be used to express IL-15 and/or IL-15Rα include mammalian cell systems infected with virus (e.g., vaccinia virus, adenovirus, retroviruses, lentiviruses, etc.); insect cell systems infected with virus (e.g., baculovirus); microorganisms such as yeast containing yeast vectors, or bacteria transformed with bacteriophage, DNA, plasmid DNA, or cosmid DNA; and stable cell lines generated by transformation using a selectable marker. In some embodiments, the nucleic acid constructs include a selectable marker gene including, but not limited to, neomycin (neo), dihydrofolate reductase (dhfr) and hygromycin (hyg).

The nucleic acid constructs can be monocistronic or multicistronic. A multicistronic nucleic acid construct may encode 2, 3, 4, 5, 6, 7, 8, 9, 10 or more, or in the range of 2-5, 5-10 or 10-20 genes/nucleotide sequences. For example, a bicistronic nucleic acid construct may comprise in the following order a promoter, a first gene (e.g., IL-15), and a second gene and (e.g., IL-15Rα). In such a nucleic acid construct, the transcription of both genes is driven by the promoter, whereas the translation of the mRNA from the first gene is by a cap-dependent scanning mechanism and the translation of the mRNA from the second gene is by a cap-independent mechanism, e.g., by an IRES.

Techniques for practicing these aspects will employ, unless otherwise indicated, conventional techniques of molecular biology, microbiology, and recombinant DNA manipulation and production, which are routinely practiced by one of skill in the art. See, e.g., Sambrook, 1989, Molecular Cloning, A Laboratory Manual, Second Edition; DNA Cloning, Volumes I and II (Glover, Ed. 1985); Oligonucleotide Synthesis (Gait, Ed. 1984); Nucleic Acid Hybridization (Hames & Higgins, Eds. 1984); Transcription and Translation (Hames & Higgins, Eds. 1984); Animal Cell Culture (Freshney, Ed. 1986); Immobilized Cells and Enzymes (IRL Press, 1986); Perbal, A Practical Guide to Molecular Cloning (1984); Gene Transfer Vectors for Mammalian Cells (Miller & Calos, Eds. 1987, Cold Spring Harbor Laboratory); Methods in Enzymology, Volumes 154 and 155 (Wu & Grossman, and Wu, Eds., respectively), (Mayer & Walker, Eds., 1987); Immunochemical Methods in Cell and Molecular Biology (Academic Press, London, Scopes, 1987), Expression of Proteins in Mammalian Cells Using Vaccinia Viral Vectors in Current Protocols in Molecular Biology, Volume 2 (Ausubel et al., Eds., 1991).

In a specific embodiment, the nucleic acid constructs encoding IL-15 or IL-15Rα can be co-transfected or transfected into the same host cells or different host cells. Optionally, a nucleic acid construct comprising nucleic acids encoding a selectable marker gene can also be transfected into the same cells to select for transfected cells. If the nucleic acid constructs comprising nucleic acids encoding IL-15 and IL-15Rα are transfected into different cells, IL-15 and IL-15Rα expressed by the different cells can be isolated and contacted with each other under conditions suitable to form IL-15/IL-15Rα complexes described in above. Any techniques known to one of skill in the art can be used to transfect or transducer host cells with nucleic acids including, e.g., transformation, transfection, conjugation, protoplast fusion, electroporation, calcium phosphate-precipitation, direct microinjection, and infection with viruses, including but not limited to adenoviruses, lentiviruses, and retroviruses.

For long-term, high-yield production of recombinant IL-15 and IL-15Rα polypeptides, stable cell lines can be generated. For example, cell lines can be transformed using the nucleic acid constructs described herein which may contain a selectable marker gene on the same or on a separate nucleic acid construct. The selectable marker gene can be introduced into the same cell by co-transfection. Following the introduction of the vector, cells are allowed to grow for 1-2 days in an enriched media before they are switched to selective media to allow growth and recovery of cells that successfully express the introduced nucleic acids. Resistant clones of stably transformed cells may be proliferated using tissue culture techniques well known in the art that are appropriate to the cell type. In a particular embodiment, the cell line has been adapted to grow in serum-free medium. In one embodiment, the cell line has been adapted to grow in serum-free medium in shaker flasks. In one embodiment, the cell line has been adapted to grow in stir or rotating flasks. In certain embodiments, the cell line is cultured in suspension. In particular embodiments, the cell line is not adherent or has been adapted to grow as nonadherent cells. In certain embodiments, the cell line has been adapted to grow in low calcium conditions. In some embodiments, the cell line is cultured or adapted to grow in low serum medium.

In a specific embodiment, a particularly preferred method of high-yield production of a recombinant polypeptide of the present invention is through the use of dihydro folate reductase (DHFR) amplification in DHFR-deficient CHO cells, by the use of successively increasing levels of methotrexate as described in U.S. Pat. No. 4,889,803. The polypeptide obtained from such cells may be in a glycosylated form.

In one embodiment, cell lines are engineered to express the stable heterodimer of native human IL-15 and native soluble human IL-15Rα, which can then be purified, and administered to a human. In one embodiment, the stability of the IL-15/IL-15Rα heterodimer is increased when produced from cell lines recombinantly expressing both IL-15 and IL-15Rα.

In a specific embodiment, the host cell recombinantly expresses IL-15 and the full length IL-15Rα. In another specific embodiment, the host cell recombinantly expresses IL-15 and the soluble form of IL-15Rα. In another specific embodiment, the host cell recombinantly expresses IL-15 and a membrane-bound form of IL-15Rα which is not cleaved from the surface of the cell and remains cell associated. In some embodiments, the host cell recombinantly expressing IL-15 and/or IL-15Rα (full-length or soluble form) also recombinantly expresses another polypeptide (e.g., a cytokine or fragment thereof).

In certain embodiments, such a host cell recombinantly expresses an IL-15 polypeptide in addition to an IL-15Rα polypeptide. The nucleic acids encoding IL-15 and/or IL-15Rα can be used to generate mammalian cells that recombinantly express IL-15 and IL-15Rα in high amounts for the isolation and purification of IL-15 and IL-15Rα, preferably the IL-15 and the IL-15Rα are associated as complexes. In one embodiment, high amounts of IL-15/IL-15Rα complexes refer to amounts of IL-15/IL-15Rα complexes expressed by cells that are at least 1 fold, 2 fold, 3 fold, 4 fold, 5 fold, 6 fold, 7 fold, 8 fold, 9 fold, 10 fold, 20 fold, or more than 20 fold higher than amounts of IL-15/IL-15Rα complexes expressed endogenously by control cells (e.g., cells that have not been genetically engineered to recombinantly express IL-15, IL-15Rα, or both IL-15 and IL-15Rα, or cells comprising an empty vector). In some embodiments, a host cell described herein expresses approximately 0.1 pg to 25 pg, 0.1 pg to 20 pg, 0.1 pg to 15 pg, 0.1 pg to 10 pg, 0.1 pg to 5 pg, 0.1 pg to 2 pg, 2 pg to 10 pg, or 5 to 20 pg of IL-15 as measured by a technique known to one of skill in the art (e.g., an ELISA). In certain embodiments, a host cell described herein expresses approximately 0.1 to 0.25 pg per day, 0.25 to 0.5 pg per day, 0.5 to 1 pg per day, 1 to 2 pg per day, 2 to 5 pg per day, or 5 to 10 pg per day of IL-15 as measured by a technique known to one of skill in the art (e.g., an ELISA). In a specific embodiment, the IL-15Rα is the soluble form of IL-15Rα. In a specific embodiment, the IL-15Rα is the soluble form of IL-15Rα associated with IL-15 in a stable heterodimer, which increases yields and simplifies production and purification of bioactive heterodimer IL-15/soluble IL-15Rα cytokine.

Recombinant IL-15 and IL-15Rα can be purified using methods of recombinant protein production and purification are well known in the art, e.g., see International Publication No. WO 2007/070488. Briefly, the polypeptide can be produced intracellularly, in the periplasmic space, or directly secreted into the medium. Cell lysate or supernatant comprising the polypeptide can be purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™ (gel filtration substance; Pharmacia Inc., Piscataway, N.J.) chromatography on an anion or cation exchange resin (such as a polyaspartic acid column), chromatofocusing, SDS-PAGE, and ammonium sulfate precipitation are also available.

In some embodiments, IL-15 and IL-15Rα are synthesized or recombinantly expressed by different cells and subsequently isolated and combined to form an IL-15/IL-15Rα complex, in vitro, prior to administration to a subject. In other embodiments, IL-15 and IL-15Rα are synthesized or recombinantly expressed by different cells and subsequently isolated and simultaneously administered to a subject an IL-15/IL-15Rα complex in situ or in vivo. In yet other embodiments, IL-15 and IL-15Rα are synthesized or expressed together by the same cell, and the IL-15/IL-15Rα complex formed is isolated.

The host cells chosen for expression of nucleic acids will depend upon the intended use of the cells. Factors such as whether a cell glycosylates similar to cells that endogenously express, e.g., IL-15 and/or IL-15Rα, may be considered in selecting the host cells.

Non-limiting examples of host cells that can be used to express the protein(s) encoded by the nucleic acid constructs herein include mammalian cells, bacterial cells, yeast cells, primary cells, immortalized cells, plant cells and insect cells. In a specific embodiment, the host cells are a mammalian cell line. Examples of mammalian cell lines include, but are not limited to, COS, CHO, HeLa, NIH3T3, HepG2, MCF7, HEK 293, HEK 293T, RD, PC12, hybridomas, pre-B cells, 293, 293H, K562, SkBr3, BT474, A204, M07Sb, TFβ1, Raji, Jurkat, MOLT-4, CTLL-2, MC-IXC, SK-N-MC, SK-N-MC, SK-N-DZ, SH-SY5Y, C127, NO, and BE(2)-C cells. Other mammalian cell lines available as hosts for expression are known in the art and include many immortalized cell lines available from the American Type Culture Collection (ATCC).

CHO Cell

Chinese hamster ovary (CHO) cells are most commonly used for the production of glycosylated polypeptides for therapeutic use. These cells produce a defined glycosylation profile and allow the creation of genetically stable, high-productivity cell lines. Furthermore, CHO cells can be cultured at high cell density in serum-free media to develop safe and reproducible biological processes. One major problem that is encountered when using CHO cells as host cells for recombinant expression is the proteolytic degradation of the expressed and secreted polypeptide of interest in the cell culture medium, also referred to as “clipping”.

In some embodiments, the IL-15/IL-15Rα complex is produced by a CHO cell line that has been altered to impare the function of matripase, e.g. by reducing or eliminating the functional expression of the matriptase gene, significantly decreasing the proteolytic degradation (“clipping”) of a recombinant polypeptide of interest that is expressed and secreted by said cell into the cell culture medium. Thus, impairing the effect of matriptase in said cell reduces clipping of the secreted recombinant IL-15/IL-15Rα complex, compared to a corresponding vertebrate cell in which the effect of matriptase is not impaired. With matriptase, a key protease responsible for clipping of recombinantly expressed and secreted polypeptides was identified. Altering the vertebrate cell to impair the effect of matriptase allows to significantly improve the recombinant production of a polypeptide of interest by reducing or eliminating clipping of the recombinantly expressed and secreted polypeptide of interest in the cell culture medium. Thereby, the yield of IL-15/IL-15Rα complex is increased.

In CHO cells, the enzyme responsible for producing alpha(2-6) linked NANA extensions to core glycan structures (Beta-galactoside alpha-2,6-sialyltransferase 1) has been reported as inactive or not-expressed in CHO cells (see e.g. Chung et al 2017)—although the gene for the enzyme itself is present in C. griseus. Yet it was unexpectedly discovered that that the CHO cell produced IL-15/IL-15Rα complex of the present disclosure has different glycosylation patterns compared to the IL-15/IL-15Rα complex produced by human cell lines. In addition, the alpha(2-6) linkage type glycan was observed in the CHO cell produced IL-15/IL-15Rα complex. This glycosylation pattern is unique. It may potentially provide direct benefits compared to the expected CHO pattern by being closer to the human glycosylation forms. Details of the CHO cell line can be found in WO2015/166427, which is incorporated herein as a reference.

Pharmaceutical Composition

Provided herein are compositions comprising an IL-15/IL-15Rα complex. The compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. The compositions (e.g., pharmaceutical compositions) comprise an effective amount of an IL-15/IL-15Rα complex, or a combination of an IL-15/IL-15Rα complex and a pharmaceutically acceptable carrier. In specific embodiments, the compositions (e.g., pharmaceutical compositions) comprise an effective amount of one or more IL-15/IL-15Rα complexes and a pharmaceutically acceptable carrier. In some embodiments, the composition further comprises an additional therapeutic, e.g., anti-cancer agent, anti-viral agent, anti-inflammatory agent, adjuvant. Non-limiting examples of such therapeutics are provided infra.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or, more preferably, MF59C.1 adjuvant), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In one embodiment, water is a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Pharmaceutical compositions may be formulated in any conventional manner using one or more pharmaceutically acceptable carriers or excipients. In a specific embodiment, an IL-15/IL-15Rα complex administered to a subject in accordance with the methods described herein is administered as a pharmaceutical composition.

Generally, the components of the pharmaceutical compositions comprising an IL-15/IL-15Rα complex is supplied in unit dosage form, for example, as a dry lyophilized powder or water free concentrate in a hermetically sealed container such as an ampoule or sachette indicating the quantity of active agent. Where the IL-15/IL-15Rα complex is to be administered by infusion, it can be dispensed with an infusion bottle containing sterile pharmaceutical grade water or saline (e.g., PBS). Where the IL-15/IL-15Rα complex is administered by injection, an ampoule of sterile water for injection or saline can be provided so that the ingredients may be mixed prior to administration.

In some embodiments, the IL-15/IL-15Rα complex may be formulated for administration by any method known to one of skill in the art, including but not limited to, parenteral (e.g., subcutaneous, intravenous, intratumoral or intramuscular) administration. In one embodiment, the IL-15/IL-15Rα complex is formulated for local or systemic parenteral administration, for example intratumoral administration. In a specific embodiment, the IL-15/IL-15Rα complex is formulated for subcutaneous or intravenous administration, respectively. In one embodiment, the IL-15/IL-15Rα complex is formulated in a pharmaceutically compatible solution.

The IL-15/IL-15Rα complex can be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Treatment and Dose Regimen

In one aspect, provided herein are methods for enhancing IL-15-mediated immune function, comprising administering to subjects complexes IL-15/IL-15Rα complexes in a specific dose regimen. Since enhancing IL-15-mediated immune function is beneficial for the prevention, treatment and/or management of certain disorders, provided herein are methods for the prevention, treatment and/or management of such disorders comprising administering to a subject in need thereof IL-15/IL-15Rα complexes. Non-limiting examples of disorders in which it is beneficial to enhance IL-15-mediated immune function include cancer, lymphopenia, immunodeficiencies, infectious diseases, and wounds.

In one embodiment, provided herein is a method for preventing, treating and/or managing disorders in a subject, wherein enhancement of IL-15-mediated immune function is beneficial for the prevention, treatment and/or management of such disorders, the method comprising administering the same dose of an IL-15/IL-15Rα complex to a subject for the duration of the treatment cycle. In one embodiment, the dose is in the range of 0.1 μg/kg and 0.5 μg/kg. In one embodiment, the dose is in the range of 0.25 μg/kg and 1 μg/kg. In a specific embodiment, the dose is in the range of 0.5 μg/kg and 2 μg/kg. In another embodiment, the dose is between 1 μg/kg and 4 μg/kg. In another embodiment, the dose is between 2 μg/kg and 8 μg/kg. In another embodiment, the dose is 0.1 μg/kg, 0.25 μg/kg, 0.5 μg/kg, 1 μg/kg, 2 μg/kg, 4 μg/kg, 5 μg/kg, 6 μg/kg, 8 μg/kg. In a specific embodiment, the dose is 1 μg/kg. In certain embodiments, the dose is administered 1, 2, 3, 4, 5, 6, 7, 8, 9, 10 or more times, or 1 to 3, 1 to 4, 2 to 4, 2 to 5, 2 to 6, 3 to 6, 4 to 6, 6 to 8, 5 to 8, or 5 to 10 times. In some embodiments, the dose is administered 1, 2, 3, 4, 5, 6, 7, 8, 9, 10 or more times, or 1 to 3, 1 to 4, 2 to 4, 2 to 5, 1 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In specific embodiments, each dose is administered at least 1, 2, 3, 4, 5, 6 or more times over a 5 to 7 day, 5 to 10 day, 7 to 12 day, 7 to 14 day, 7 to 21 day or 14 to 21 day period of time. In another specific embodiment, each dose is administered at least once and the subject is administered a dose once per week for a three week period.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Rα complex to the subject in a dosing regimen at least once, twice, four times or six times in a dosing cycle before a period of non-administration. In a specific embodiment the IL-15/IL-15Rα complex is administered once a week for three weeks with no administration in week four. The dosing cycle is then repeated.

In an alternative embodiment, provided herein is a method for preventing, treating and/or managing disorders in a subject, wherein enhancement of IL-15-mediated immune function is beneficial for the prevention, treatment and/or management of such disorders, the method comprising (a) administering at least one initial low dose of an IL-15/IL-15Rα complex to a subject; and (b) administering successively higher doses of the IL-15/IL-15Rα complex to the subject for the duration of the treatment cycle. In a specific embodiment, provided herein is a method for preventing, treating and/or managing cancer in a subject, method comprising (a) administering an initial dose of an IL-15/IL-15Rα complex to the subject for the duration of the treatment cycle; and (b) administering successively higher doses of the IL-15/IL-15Rα complex to the subject for the duration of the treatment cycle. In a specific embodiment, the initial dose is in the range of 0.1 μg/kg and 0.5 μg/kg. In a specific embodiment, the initial dose is in the range of 0.25 μg/kg and 1 μg/kg. In another embodiment, the initial dose is in the range of 0.5 μg/kg and 2 μg/kg. In a specific embodiment, the initial dose is between 1 μg/kg and 4 μg/kg. In another embodiment, the initial dose is between 2 μg/kg and 8 μg/kg. In another embodiment, the initial dose is about 0.25 μg/kg. In another embodiment, the initial dose is about 0.5 μg/kg. In another embodiment, the initial dose is about 1 μg/kg. In another embodiment, the initial dose is 0.1 μg/kg, 0.25 μg/kg, 0.5 μg/kg, 1 μg/kg, 2 μg/kg, 4 μg/kg, 5 μg/kg, 6 μg/kg, 8 μg/kg. In certain embodiments, the initial dose is administered 1, 2, 3, 4, 5, 6, 7, 8, 9, 10 or more times, or 1 to 3, 1 to 4, 2 to 4, 2 to 5, 2 to 6, 3 to 6, 4 to 6, 6 to 8, 5 to 8, or 5 to 10 times. In some embodiments, the initial dose is administered 1, 2, 3, 4, 5, 6, 7, 8, 9, 10 or more times, or 1 to 3, 1 to 4, 2 to 4, 2 to 5, 1 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, each successively higher dose is 1.2, 1.25, 1.3, 1.35, 1.4, 1.45, 1.5, 2, 2.5, 3, 3.5, 4, 4.5, 5, 5.5, or 6 times higher than the previous dose, or 1.2 to 2, 2 to 3, 2 to 4, 1 to 5, 2 to 6, 3 to 4, 3 to 6, or 4 to 6 times higher than the previous dose, or 2 times higher than the previous dose. In some embodiments, each successively higher dose is 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% higher than the previous dose. In specific embodiments, each dose is administered at least 1, 2, 3, 4, 5, 6 or more times over a 5 to 7 day, 5 to 10 day, 7 to 12 day, 7 to 14 day, 7 to 21 day or 14 to 21 day period of time. In another specific embodiment, each dose is administered at least once and the subject is administered a dose three times per 7 day week (e.g., Monday, Wednesday and Friday) for a two week period.

In certain embodiments, the subject is monitored for the following adverse events, such as grade 3 or 4 thrombocytopenia, grade 3 or 4 granulocytopenia, grade 3 or 4 leukocytosis (White Blood Cell (WBC)>100,000 mm³), grade 3 or 4 decreases in WBC, absolute lymphocyte count (ALC) and/or absolute neutrophil count (ANC), lymphocytosis and organ dysfunction (e.g., liver or kidney dysfunction). In certain embodiments, the dose is not increased and the dose may be remain the same, be stopped or reduced if the subject experiences adverse events, such as grade 3 or 4 thrombocytopenia, grade 3 or 4 granulocytopenia, grade 3 or leukocytosis (White Blood Cell>100,000 mm³), grade 3 or 4 decreases in WBC, absolute lymphocyte count (ALC) and/or absolute neutrophil count (ANC), lymphocytosis, and organ dysfunction (e.g., liver or kidney dysfunction). In accordance with these embodiments, the dose of the IL-15/IL-15Rα complex administered to the subject may be reduced or remain the same until the adverse events decrease or disappear.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Rα complex to the human subject in a dose regimen beginning with a first cycle comprising an initial dose of between 0.25 μg/kg and 4 μg/kg, and sequential cycles wherein the dose is increased two to three times over the previous dose. Each dose is administered at least once, twice, four times or six times before elevating the dose to the next level, and the concentration of free IL-15 in a sample (e.g., a plasma sample) obtained from the subject a certain period of time after the administration of a dose of the IL-15/IL-15Rα complex (e.g., approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Rα complex and before the administration of another dose of the IL-15/IL-15Rα complex) is monitored before elevating the dose to the next level.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Rα complex to the subject in a dose regimen at the following sequential doses: (i) 0.25 μg/kg; (ii) 0.5 μg/kg; (iii) 1 μg/kg; (iv) 2 μg/kg; (v) 4 μg/kg; and (vi) 8 μg/kg. In a certain embodiment, the IL-15/IL-15Rα complex is administered to the subject in a dose regimen at the following sequential doses: (i) 1 μg/kg; (ii) 2 μg/kg; (iii) 4 μg/kg; and (iv) 8 μg/kg. Each dose is administered at least once, twice, four times or six times in a dosing cycle before elevating the dose to the next level, and wherein the concentration of free IL-15 in a sample (e.g., a plasma sample) obtained from the subject a certain period of time after the administration of a dose of the IL-15/IL-15Rα complex (e.g., approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Rα complex and before the administration of another dose of the IL-15/IL-15Rα complex) is monitored before elevating the dose to the next level.

In another embodiment, provided herein is a method for preventing, treating and/or managing cancer in a subject, method comprising administering an IL-15/IL-15Rα complex to the subject in an dose regimen at the following sequential doses: (i) 1 μg/kg; (ii) 2 μg/kg; (iii) 4 μg/kg; and (iv) 8 μg/kg, wherein each dose is administered at least at least once, twice, four times or six times in a dosing cycle before elevating the dose to the next level. In a specific embodiment, the method comprises administering the IL-15/IL-15Rα complex to the subject using a cyclical administration regimen, wherein the cyclical administration regimen comprises: (a) administering subcutaneously to the subject a dose of 0.1 to 10m/kg of the IL-15/IL-15Rα complex every 1, 2 or 3 days over a first period of 1 week to 3 weeks; and (b) after a second period of 1 week to 2 months in which no IL-15/IL-15Rα complex is administered to the subject, administering subcutaneously to the subject a dose of 0.1 to 10 μg/kg of the IL-15/IL-15Rα complex every 1, 2 or 3 days over a third period of 1 week to 3 weeks.

In a particular embodiment, the subject is a human subject. In certain embodiments, the dose in the treatment cycle is administered 1, 2, 3, 4, 5, 6, 7, 8, 9, 10 or more times, or 1 to 3, 1 to 4, 1 to 5,2 to 4,2 to 5, 1 to 6,2 to 6, 1 to 6,3 to 6,4 to 6,6 to 8, 5 to 8, or 5 to 10 times. In some embodiments, the dose is administered 1, 2, 3, 4, 5, 6, 7, 8, 9, 10 or more times, or 1 to 3, 1 to 4, 1 to 5, 2 to 4, 2 to 5, 2 to 6, 1 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, each dose is administered 1, 2, 3, 4, 5, 6, 7, 8, 9, 10 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times, per dosing cycle. In specific embodiments, each dose is administered at least 1, 2, 3, 4, 5, 6 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times over a 5 to 7 day, 5 to 10 day, 7 to 12 day, 7 to 14 day, 7 to 21 day or 14 to 21 day period of time.

In another specific embodiment, the subject is administered a dose three times per 7 day week (e.g., Monday, Wednesday and Friday). In certain embodiments, the subject is monitored for the following adverse events, such as grade 3 or 4 thrombocytopenia, grade 3 or 4 granulocytopenia, grade 3 or 4 leukocytosis (White Blood Cell (WBC)>100,000 mm3), grade 3 or 4 decreases in WBC, absolute lymphocyte count (ALC) and/or absolute neutrophil count (ANC), lymphocytosis, and organ dysfunction (e.g., liver or kidney dysfunction). In certain embodiments, the dose is not increased and the dose may be remain the same, be stopped or reduced if the subject experiences adverse events, such as grade 3 or 4 thrombocytopenia, grade 3 or 4 granulocytopenia, grade 3 or leukocytosis (White Blood Cell>100,000 mm3), grade 3 or 4 decreases in WBC, absolute lymphocyte count (ALC) and/or absolute neutrophil count (ANC), lymphocytosis, and organ dysfunction (e.g., liver or kidney dysfunction). In accordance with these embodiments, the dose of the IL-15/IL-15Rα complex administered to the subject may be reduced or remain the same until the adverse events decrease or disappear.

In specific embodiments, in accordance with the methods described herein, each dose is administered once a week for three weeks. In specific embodiments, in accordance with the methods described herein, each dose is administered once, three times a week for two weeks. In specific embodiments, in accordance with the methods described herein, each dose is administered once, three times a week for two, three, or four weeks. In specific embodiments, in accordance with the methods described herein, each dose is administered once, six times a week for two, three, or four weeks. In specific embodiments, in accordance with the methods described herein, each dose is administered once, every other day, for two, three, or four weeks. In specific embodiments, in accordance with the methods described herein, each dose is administered once, every day, for two, three, or four weeks.

In certain embodiments, the IL-15/IL-15Rα complex is administered subcutaneously to a subject in accordance with the methods described herein. In some embodiments, the IL-15/IL-15Rα complex is administered intravenously or intramuscularly to a subject in accordance with the methods described herein. In certain embodiments, the IL-15/IL-15Rα complex is administered intratumorally to a subject in accordance with the methods described herein. In some embodiments, the IL-15/IL-15Rα complex is administered locally to a site (e.g., a site of infection) in a subject in accordance with the methods described herein.

In certain embodiments, a sample obtained from a subject in accordance with the methods described herein is a blood sample. In a specific embodiment, the sample is a plasma sample. Basal plasma levels of IL-15 are approximately 1 pg/ml in humans, approximately 8-10 pg/ml in monkeys (such as macaques), and approximately 12 pg/m in rodents (such as mice). Techniques known to one skilled in the art can be used to obtain a sample from a subject.

In specific embodiments, examples of immune function enhanced by the methods described herein include the proliferation/expansion of lymphocytes (e.g., increase in the number of lymphocytes), inhibition of apoptosis of lymphocytes, activation of dendritic cells (or antigen presenting cells), and antigen presentation. In particular embodiments, an immune function enhanced by the methods described herein is proliferation/expansion in the number of or activation of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells). In one embodiment, the methods described herein enhance the proliferation/expansion or number of lymphocyte progenitors. In some embodiments, the methods described herein increases the number of CD4+ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells) by approximately 1 fold, 2 fold, 3 fold, 4 fold, 5 fold, 6 fold, 7 fold, 8 fold, 9 fold, 10 fold, 20 fold, or more relative to a negative control.

In a specific embodiment, the methods described herein enhance or induce immune function in a subject by at least 0.2 fold, 0.5 fold, 0.75 fold, 1 fold, 1.5 fold, 2 fold, 2.5 fold, 3 fold, 4 fold, 5 fold, 6 fold, 7 fold, 8 fold 9 fold, or at least 10 fold relative to the immune function in a subject not administered the combination of an IL-15/IL-15Rα complex and an anti-PD-1 antibody molecule using assays well known in the art, e.g., ELISPOT, ELISA, and cell proliferation assays. In a specific embodiment, the methods described herein enhance or induce immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the combination of an IL-15/IL-15Rα complex and an anti-PD-1 antibody molecule using assays well known in the art, e.g., ELISPOT, ELISA, and cell proliferation assays. In a specific embodiment, the immune function is cytokine release (e.g., interferon-gamma, IL-2, IL-5, IL-10, IL-12, or transforming growth factor (TGF)-beta). In one embodiment, the IL-15 mediated immune function is NK cell proliferation, which can be assayed, e.g., by flow cytometry to detect the number of cells expressing markers of NK cells (e.g., CD56). In one embodiment, the IL-15 mediated immune function is CD8+ T cell proliferation, which can be assayed, e.g., by flow. In another embodiment, the IL-15 mediated immune function is antibody production, which can be assayed, e.g., by ELISA. In some embodiments, the IL-15 mediated immune function is effector function, which can be assayed, e.g., by a cytotoxicity assay or other assays well known in the art. The effect of one or more doses of a combination of an IL-15/IL-15Rα complex and an anti-PD-1 antibody molecule on peripheral blood lymphocyte counts can be monitored/assessed using standard techniques known to one of skill in the art. Peripheral blood lymphocytes counts in a mammal can be determined by, e.g., obtaining a sample of peripheral blood from said mammal, separating the lymphocytes from other components of peripheral blood such as plasma using, e.g., FicollHypaque (Pharmacia) gradient centrifugation, and counting the lymphocytes using trypan blue. Peripheral bloodT-cell counts in mammal can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS. Further, the effect on a particular subset of T cells (e.g., CD2⁺, CD4⁺, CD8⁺, CD4⁺RO⁺, CD8⁺RO⁺, CD4⁺RA⁺, or CD8⁺RA⁺) or NK cells can be determined using standard techniques known to one of skill in the art such as FACS.

The plasma levels of IL-15 and/or PD-1 can be assessed using standard techniques known to one of skill in the art. For example, plasma can be obtained from a blood sample obtained from a subject and the levels of IL-15 and/or PD-1 in the plasma can be measured by ELISA.

Combination Therapy

Other therapies that can be used in combination with IL-15/IL-15Rα are also provided by the present disclosure. In one aspect, provided herein are methods for preventing, treating, and/or managing cancer, comprising administering an effective amount of an IL-15/IL-15Rα complex and an anti-PD-1 antibody molecule or a composition comprising an IL-15/IL-15Rα complex and an anti-PD-1 antibody molecule to a subject in need thereof. As used herein, the term “cancer” is meant to include all types of cancerous growths or oncogenic processes, metastatic tissues or malignantly transformed cells, tissues, or organs, irrespective of histopathologic type or stage of invasiveness. In a specific embodiment, the IL-15/IL-15Rα complex is administered subcutaneously either at the same, repeated dose or alternatively in a dose escalation regimen. In a specific embodiment, anti-PD-1 antibody molecule is administered as an intravenous infusion in a flat dosing regimen.

In specific embodiments, the administration of a combination of an IL-15/IL-15Rα complex and an anti-PD-1 antibody molecule to a subject in accordance with the methods described herein achieves one, two, or three or more results: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than 10%, or by more than 8%, or by more than 6%, or by more than 4%; preferably the size of the tumor does not increase by more than 2%.

In a specific embodiment, the administration of a combination of an IL-15/IL-15Rα complex and an anti-PD-1 antibody molecule to a subject with cancer (in some embodiments, an animal model for cancer) in accordance with the methods described herein inhibits or reduces the growth of a tumor by at least 2 fold, preferably at least 2.5 fold, at least 3 fold, at least 4 fold, at least 5 fold, at least 7 fold, or at least 10 fold relative to the growth of a tumor in a subject with cancer (in some embodiments, in the same animal model for cancer) administered a negative control as measured using assays well known in the art. In another embodiment, the administration of a combination of an IL-15/IL-15Rα complex and an anti-PD-1 antibody molecule to a subject with cancer (in some embodiments, an animal model for cancer) in accordance with the methods described herein inhibits or reduces the growth of a tumor by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to the growth of a tumor in a subject with cancer (in some embodiments, in the same animal model for cancer) administered a negative control, or an IL-15/IL-15Rα complex or an anti-PD-1 antibody molecule as a single agent, as measured using assays well known in the art.

Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the combination an IL-15/IL-15Rα complex and an anti-PD-1 antibody molecule disclosed herein include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the combination therapy described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodisplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers.

In a specific embodiment, the cancer is melanoma, renal cancer, colon cancer, or prostate cancer. In another embodiment, the cancer is metastatic. In another embodiments, the subject has been previously treated with immune checkpoint inhibitor (CPI), for example, anti PD-1/PD-L1, and anti CTLA-4, and has responded and progressed.

The combination of an IL-15/IL-15Rα complex and an anti-PD-1 antibody molecule can be administered together with one or more other therapies, e.g., anti-cancer agents, cytokines or anti-hormonal agents, to treat and/or manage cancer. Non-limiting examples anti-cancer agents are described below.

In one embodiment, provided herein is a method for preventing, treating and/or managing disorders in a subject, e.g., a hyperproliferative condition or disorder (e.g., a cancer) in a subject including administering to a subject an anti-PD-1 antibody molecule. In some embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment, the anti-PD-1 antibody molecule is administered at a dose from about 300 mg to 400 mg once every three weeks or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks.

In accordance with the methods described herein, the IL-15/IL-15Rα complex may be administered to a subject in a pharmaceutical composition. In specific embodiments, the IL-15/IL-15Rα complex is administered in combination with one or more other therapies, e.g., an anti-PD-1 antibody molecule. Combination therapy includes concurrent and successive administration of an IL-15/IL-15Rα complex and an anti-PD-1 antibody molecule. As used herein, the IL-15/IL-15Rα complex and the anti-PD-1 antibody molecule are said to be administered concurrently if they are administered to the patient on the same day, for example, simultaneously, or 1, 2, 3, 4, 5, 6, 7, or 8 hours apart. In contrast, the IL-15/IL-15Rα complex and the anti-PD-1 antibody molecule are said to be administered successively if they are administered to the patient on the different days, for example, the IL-15/IL-15Rα complex and the anti-PD-1 antibody molecule can be administered at a 1-day, 2-day or 3-day interval. In the methods described herein, administration of the IL-15/IL-15Rα complex can precede or follow administration of the anti-PD-1 antibody molecule. When administered simultaneously, the IL-15/IL-15Rα complex and the anti-PD-1 antibody molecule can be in the same pharmaceutical composition or in a different pharmaceutical composition.

The combination of an IL-15/IL-15Rα complex and an anti-PD-1 antibody molecule can also be administered together with radiation therapy comprising, e.g., the use of x-rays, gamma rays and other sources of radiation to destroy the cancer cells. In specific embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass. An IL-15/IL-15Rα complex and an anti-PD-1 antibody molecule can also be administered in combination with chemotherapy. In one embodiment, an IL-15/IL-15Rα complex and an anti-PD-1 antibody molecule can be administered in accordance with the methods described herein before, during or after radiation therapy or chemotherapy. In one embodiment, a combination of an IL-15/IL-15Rα complex and an anti-PD-1 antibody molecule can be administered before, during or after surgery.

In some embodiments, the combination of an IL-15/IL-15Rα complex and an anti-PD-1 antibody molecule is administered to a subject suffering from or diagnosed with cancer. In other embodiments, the combination of an IL-15/IL-15Rα complex and an anti-PD-1 antibody molecule is administered to a subject predisposed or susceptible to developing cancer.

In certain embodiments, the combination of an IL-15/IL-15Rα complex and an anti-PD-1 antibody molecule is administered to a subject which is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In other embodiments, the combination of an IL-15/IL-15Rα complex and an anti-PD-1 antibody molecule is administered to a human adult. In certain embodiments, the combination of an IL-15/IL-15Rα complex and an anti-PD-1 antibody molecule is administered to a subject that is, will or has undergone surgery, chemotherapy and/or radiation therapy. In some embodiments, the combination of an IL-15/IL-15Rα complex and an anti-PD-1 antibody molecule is administered to refractory patients. In a certain embodiment, refractory patient is a patient refractory to a standard anti-cancer therapy. In certain embodiments, a patient with cancer, is refractory to a therapy when the cancer has not significantly been eradicated and/or the symptoms have not been significantly alleviated. The determination of whether a patient is refractory can be made either in vivo or in vitro by any method known in the art for assaying the effectiveness of a treatment, using art-accepted meanings of “refractory” in such a context. In various embodiments, a patient with cancer is refractory when a cancerous tumor has not decreased or has increased.

Other methods of the invention are used to treat patients that have been exposed to particular toxins or pathogens. Accordingly, another aspect of the invention provides a method of treating an infectious disease in a subject comprising administering to the subject a combination as disclosed herein, e.g., a combination including an IL-15/IL-15Rα complex and an anti-PD-1 antibody molecule, such that the subject is treated for the infectious disease.

In the treatment of infection (e.g., acute and/or chronic), administration of the combination of an IL-15/IL-15Rα complex and an anti-PD-1 antibody molecule can be combined with conventional treatments in addition to or in lieu of stimulating natural host immune defenses to infection. Natural host immune defenses to infection include, but are not limited to inflammation, fever, antibody-mediated host defense, T-lymphocyte-mediated host defenses, including lymphokine secretion and cytotoxic T-cells (especially during viral infection), complement mediated lysis and opsonization (facilitated phagocytosis), and phagocytosis. The ability of the anti-PD-1 antibody molecules to reactivate dysfunctional T-cells would be useful to treat chronic infections, in particular those in which cell-mediated immunity is important for complete recovery.

Antibody mediated PD-1 blockade can act as an adjuvant to IL-15/IL-15Rα complex administration or in combination with an Il-15/IL-15Rα complexes and/or vaccines, to stimulate the immune response to pathogens, toxins and self-antigens. Examples of pathogens for which this therapeutic approach may be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas Aeruginosa. Immune system stimulation by IL-15/IL-15Rα complexes and PD-1 blockade is particularly useful against established infections by agents such as HIV that present altered antigens over the course of the infections. These novel epitopes are recognized as foreign at the time of treatment, thus provoking a strong T cell response that is not dampened by negative signals through PD-1, for example.

Other therapies that can be used in combination with an IL-15/IL-15Rα complex and anti-PD-1 antibody molecule, for the prevention, treatment and/or management of a disease, e.g., cancer, infectious disease, lymphopenia, immunodeficiency and wounds, include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such therapies include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g., aspirin, ibuprofen, diclofenac, and COX-2 inhibitors), pain relievers, leukotriene antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, ganciclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythromycin, penicillin, mithramycin, and anthramycin (AMC)).

Any therapy which is known to be useful, or which has been used or is currently being used for the prevention, management, and/or treatment of a disease that is affected by IL-15 function/signaling and/or immunecheckpoint modulation can be used in combination with a combination therapy of an IL-1541-15Rα complex and anti-PD-1 antibody molecule. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N J, 1999; Cecil Textbook of Medicine, 20th Ed., Bennett and Plum (eds.), W. B. Saunders, Philadelphia, 1996, and Physicians' Desk Reference (66th ed. 2012) for information regarding therapies (e.g., prophylactic or therapeutic agents) which have been or are currently being used for preventing, treating and/or managing disease or disorder, e.g., cancer, infectious disease, lymphopenia, immunodeficiency and wounds.

Non-limiting examples of one or more other therapies that can be used in addition to a combination therapy of an IL-1541-15Rα complex and anti-PD-1 antibody molecule include immunomodulatory agents, such as but not limited to, chemotherapeutic agents and non-chemotherapeutic immunomodulatory agents. Non-limiting examples of chemotherapeutic agents include methotrexate, cyclosporin A, leflunomide, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologs, and cytoxan.

Biological Activity

In one aspect, the IL-15/IL-15Rα complex and/or anti-PD-1 antibody molecule increases an immune response that can be, e.g., an antibody response (humoral response) or a cellular immune response, e.g., cytokine secretion (e.g., interferon-gamma), helper activity or cellular cytotoxicity. In one embodiment, the increased immune response is increased cytokine secretion, antibody production, effector function, T cell proliferation, and/or NK cell proliferation. Various assays to measure such activities are well known in the art, and include enzyme-linked immunosorbent assays (ELISA; see e.g., in Section 2.1 of Current Protocols in Immunology, Coligan et al. (eds.), John Wiley and Sons, Inc. 1997), a “tetramer staining” assay to identify antigen-specific T-cells (see Altman et al., (1996), Science 274: 94-96), a mixed lymphocyte target culture assay (see e.g., in Palladino et al., (1987), Cancer Res. 47:5074-5079) and an ELISPOT assay that can be used to measure cytokine release in vitro (see, e.g., Scheibenbogen et al., (1997), Int. J. Cancer 71:932-936).

In some aspects, the immune response induced or enhanced by a combination of an IL-15/IL-15Rα complex and an anti-PD-1 antibody molecule is enhanced or increased by at least 2 fold, 3 fold, 4 fold, 5 fold, 6 fold, 7 fold, 8 fold, 9 fold, 10 fold, 11 fold, or 12 fold relative to an immune response elicited by a negative control, or by an IL-15/IL-15Rα complexes or an anti-PD-1 antibody molecule administered as a single agent, as assayed by any known method in the art. In certain embodiments, the immune response induced by the combination of an IL-15/IL-15Rα complex and an anti-PD-1 antibody molecule is enhanced by at least 0.5-2 times, at least 2-5 times, at least 5-10 times, at least 10-50 times, at least 50-100 times, at least 100-200 times, at least 200-300 times, at least 300-400 times or at least 400-500 times relative to the immune response induced by a negative control as assayed by any known method in the art. In specific embodiments, the assay used to assess immune response measures the level of antibody production, cytokine production, or cellular cytotoxicity, and such assays are well known in the art. In some embodiments, the assay used to measure the immune response is an enzyme-linked immunosorbent assay (ELISA) that determines antibody or cytokine levels, an ELISPOT assay that determines cytokine release, or a [⁵¹Cr] release assay that determines cellular cytotoxicity.

In a specific embodiment, the combination of an IL-15/IL-15Rα complex and an anti-PD-1 antibody molecule increases the expression of IL-2 on whole blood activated by Staphylococcal enterotoxin B (SEB). For example, the IL-15/IL-15Rα complex and an anti-PD-1 antibody molecule increases the expression of IL-2 by at least about 2, 3, 4, or 5-fold, compared to the expression of IL-2 when an the IL-15/IL-15Rα complex, the anti-PD-1 antibody molecule or an isotype control (e.g., IgG4) is used alone. The additive or synergistic effect was more pronounced when the IL-15/IL-15Rα complex was administered on the same day as the anti-PD-1 antibody, rather than when the IL-15/IL-15Rα complex was administered 72 hours after administration of the anti-PD-1 antibody molecule.

In one embodiment, the proliferation or viability of cancer cells contacted with a combination of an IL-15/IL-15Rα complex and an anti-PD-1 antibody molecule is inhibited or reduced by at least 2 fold, preferably at least 2.5 fold, at least 3 fold, at least 4 fold, at least 5 fold, at least 7 fold, or at least 10 fold relative to the proliferation of the cancer cells when contacted with a negative control or an IL-15/IL-15Rα complex or an anti-PD-1 antibody molecule as a single agent, as measured using assays well known in the art, e.g., cell proliferation assays using CSFE, BrdU, and radioactive thymidine incorporation. Alternatively, cell viability can be measured by assays that measure lactate dehydrogenase (LDH), a stable cytosolic enzyme that is released upon cell lysis, or by the release of [⁵¹Cr] upon cell lysis. In another embodiment, the proliferation of cancer cells contacted with a combination of an IL-15/IL-15Rα complex and an anti-PD-1 antibody molecule is inhibited or reduced by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to cancer cells contacted with a negative control or an IL-15/IL-15Rα complex or an anti-PD-1 antibody molecule as a single agent, as measured using assays well known in the art, e.g., cell proliferation assays using CSFE, BrdU, and radioactive thymidine incorporation.

Cancer cell lines on which such assays can be performed are well known to those of skill in the art. Necrosis, apoptosis and proliferation assays can also be performed on primary cells, e.g., a tissue explant.

In one embodiment, necrotic cells are measured by the ability or inability of the cell to take up a dye such as neutral red, trypan blue, or ALAMAR™ blue (Page et al., (1993), Intl. J. of Oncology 3:473-476). In such an assay, the cells are incubated in media containing the dye, the cells are washed, and the remaining dye, reflecting cellular uptake of the dye, is measured spectrophotometrically. In another embodiment, the dye is sulforhodamine B (SRB), whose binding to proteins can be used as a measure of cytotoxicity (Skehan et al., (1990), J. Natl Cancer Inst. 82:1107-12). In yet another embodiment, a tetrazolium salt, such as MTT, is used in a quantitative colorimetric assay for mammalian cell survival and proliferation by detecting living, but not dead, cells (see, e.g., Mosmann, (1983), J. Immunol. Methods 65:55-63).

In other embodiments, apoptotic cells are measured in both the attached and “floating” compartments of the cultures. Both compartments are collected by removing the supernatant, trypsinizing the attached cells, and combining both preparations following a centrifugation wash step (10 minutes, 2000 rpm). The protocol for treating tumor cell cultures with sulindac and related compounds to obtain a significant amount of apoptosis has been described in the literature (see, e.g., Piazza et al., (1995) Cancer Research 55:3110-16). Features of this method include collecting both floating and attached cells, identification of the optimal treatment times and dose range for observing apoptosis, and identification of optimal cell culture conditions. In another embodiment, apoptosis is quantitated by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, (1999), no. 2, pp. 34-37 (Roche Molecular Biochemicals). In yet another embodiment, apoptosis can be observed morphologically.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference as applicable, unless otherwise indicated. The following Examples are presented in order to more fully illustrate the preferred embodiments of the disclosure. These examples should in no way be construed as limiting the scope of the disclosed subject matter, which is defined by the appended claims.

Specific Embodiments, Citation and References

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein; the disclosure of each such reference is hereby incorporated herein by reference in its entirety.

TABLE 2
Sequence Table

| | | |
|---|---|---|
| SEQ | | |
| ID | | |
| NO | Description | Sequence |

IL-15 related sequences

| | | |
|---|---|---|
| 1 | Human IL-15 | MRISKPHLRSISIQCYLCLLL |
| | (with signal | NSHFLTEAGIHVFILGCFSAG |
| | peptide) | LPKTEANWVNVISDLKKIEDL |
| | | IQSMHIDATLYTESDVHPSCK |
| | | VTAMKCFLLELQVISLESGDA |
| | | SIHDTVENLIILANNSLSSNG |
| | | NVTESGCKECEELEEKNIKEF |
| | | LQSFVHTVQMFINTS |
| 2 | Human IL-15 | atgagaattt cgaaaccaca |
| | DNA (with | tttgagaagt atttccatcc |
| | signal peptide) | agtgctactt gtgtttactt |
| | | ctaaacaatc attttctaac |
| | | tgaagctggc attcatatct |
| | | tcattttggg ctgtttcagt |
| | | gcagggcttc ctaaaacaga |
| | | agccaactgg |
| | | gtgaatgtaa taagtgattt |
| | | gaaaaaaatt gaagatctta |
| | | ttcaatctat gcatattgat |
| | | gctactttat atacggaaag |
| | | tgatgttcac cccagttgca |
| | | aagtaacagc aatgaagtgc |
| | | tttctcttgg agttacaagt |
| | | tatttcactt gagtccggag |
| | | atgcaagtat tcatgataca |
| | | gtagaaaatc tgatcatcct |
| | | agcaaacaac agtttgtctt |
| | | ctaatgggaa tgtaacagaa |
| | | tctggatgca aagaatgtga |
| | | ggaactggag gaaaaaaata |
| | | ttaaagaatt tttgcagagt |
| | | tttgtacata ttgtccaaat |
| | | gttcatcaac acttcttga |
| 3 | human IL-15 | atgtggctcc agagcctgct |
| | with GMCSF | actcctgggg acggtggcct |
| | signal peptide | gcagcatctc gaactgggtg |
| | | aacgtgatct cggacctgaa |
| | | gaagatcgag gacctcatcc |
| | | agtcgatgca catcgacgcg |
| | | acgctgtaca cggagtcgga |
| | | cgtccacccg tcgtgcaagg |
| | | tcacggcgat gaagtgcttc |
| | | ctcctggagc tccaagtcat |
| | | ctcgctcgag tcgggggacg |
| | | cgtcgatcca cgacacggtg |
| | | gagaacctga tcatcctggc |
| | | gaacaactcg ctgtcgtcga |
| | | acgggaacgt cacggagtcg |
| | | ggctgcaagg agtgcgagga |
| | | gctggaggag aagaacatca |
| | | aggagttcct gcagtcgttc |
| | | gtgcacatcg tccagatgtt |
| | | catcaacacg tcgtga |
| 4 | IL-15 codon | cctggccatt gcatacgttg |
| | optimized | tatccatatc ataatatgta |
| | DNA | catttatatt ggctcatgtc |
| | | caacattacc gccatgttga |
| | | cattgattat tgactagtta |
| | | ttaatagtaa tcaattacgg |
| | | ggtcattagt tcatagccca |
| | | tatatggagt tccgcgttac |
| | | ataacttacg gtaaatggcc |
| | | cgcctggctg accgcccaac |
| | | gacccccgcc cattgacgtc |
| | | aataatgacg tatgttccca |
| | | tagtaacgcc aatagggact |
| | | ttccattgac gtcaatgggt |
| | | ggagtattta cggtaaactg |
| | | cccacttggc agtacatcaa |
| | | gtgtatcata tgccaagtac |
| | | gccccctatt gacgtcaatg |
| | | atggtaaatg gcccgcctgg |
| | | cattatgccc agtacatgac |
| | | cttatgggac tttcctactt |
| | | ggcagtacat ctacgtatta |
| | | gtcatcgcta ttaccatggt |
| | | gatgcggttt tggcagtaca |
| | | tcaatgggcg tggatagcgg |
| | | tttgactcac ggggatttcc |
| | | aagtctccac cccattgacg |
| | | tcaatgggag tttgttttgg |
| | | caccaaaatc aacgggactt |
| | | tccaaaatgt cgtaacaact |
| | | ccgccccatt gacgcaaatg |
| | | ggcggtaggc gtgtacggtg |
| | | ggaggtctat ataagcagag |
| | | ctcgtttagt gaaccgtcag |
| | | atcgcctgga gacgccatcc |
| | | acgctgtttt gacctccata |
| | | gaagacaccg ggaccgatcc |
| | | agcctccgcg ggcgcgcgtc |
| | | gacaagaaat gcggatctcg |
| | | aagccgcacc tgcggtcgat |
| | | atcgatccag tgctacctgt |
| | | gcctgctcct gaactcgcac |
| | | ttcctcacgg aggccggtat |
| | | acacgtcttc atcctgggct |
| | | gcttctcggc ggggctgccg |
| | | aagacggagg cgaactgggt |
| | | gaacgtgatc tcggacctga |
| | | agaagatcga ggacctcatc |
| | | cagtcgatgc acatcgacgc |
| | | gacgctgtac acggagtcgg |
| | | acgtccaccc gtcgtgcaag |
| | | gtcacggcga tgaagtgctt |
| | | cctcctggag ctccaagtca |
| | | tctcgctcga gtcgggggac |
| | | gcgtcgatcc acgacacggt |
| | | ggagaacctg atcatcctgg |
| | | cgaacaactc gctgtcgtcg |
| | | aacgggaacg tcacggagtc |
| | | gggctgcaag gagtgcgagg |
| | | agctggagga gaagaacatc |
| | | aaggagttcc tgcagtcgtt |
| | | cgtgcacatc gtccagatgt |
| | | tcatcaacac gtcgtgaggg |
| | | cccggcgcgc cgaattcgcg |
| | | gatatcggtt aacggatcca |
| | | gatctgctgt gccttctagt |
| | | tgccagccat ctgttgtttg |
| | | cccctccccc gtgccttcct |
| | | tgaccctgga aggtgccact |
| | | cccactgtcc tttcctaata |
| | | aaatgaggaa attgcatcgc |
| | | attgtctgag taggtgtcat |
| | | tctattctgg ggggtggggt |
| | | ggggcaggac agcaaggggg |
| | | aggattggga agacaatagc |
| | | aggcatgctg gggatgcggt |
| | | gggctctatg ggtacccagg |
| | | tgctgaagaa ttgacccggt |
| | | tcctcctggg ccagaaagaa |
| | | gcaggcacat ccccttctct |
| | | gtgacacacc ctgtccacgc |
| | | ccctggttct tagttccagc |
| | | cccactcata ggacactcat |
| | | agctcaggag ggctccgcct |
| | | tcaatcccac ccgctaaagt |
| | | acttggagcg gtctctccct |
| | | ccctcatcag cccaccaaac |
| | | caaacctagc ctccaagagt |
| | | gggaagaaat taaagcaaga |
| | | taggctatta agtgcagagg |
| | | gagagaaaat gcctccaaca |
| | | tgtgaggaag taatgagaga |
| | | aatcata |
| 5 | IL-15 codon | MRISKPHLRSISIQCYLCLLL |
| | optimized | NSHFLTEAGIHVFILGCFSAG |
| | amino acid | LPKTEANWVNVISDLKKIEDL |
| | | IQSMHIDATLYTESDVHPSCK |
| | | VTAMKCFLLELQVISLESGDA |
| | | SIHDTVENLIILANNSLSSNG |
| | | NVTESGCKECEELEEKNIKEF |
| | | LQSFVHIVQMFINTS |
| 6 | Human IL- | MAPRRARGCRTLGLPALLLLL |
| | 15Rα | LLRPPATRGITCPPPMSVEHA |
| | with | DIWVKSYSLYSRERYICNSGF |
| | signal | KRKAGTSSLTECVLNKATNVA |
| | peptide | HWTTPSLKCIRDPALVHQRPA |
| | | PPSTVTTAGVTPQPESLSPSG |
| | | KEPAASSPSSNNTAATTAAIV |
| | | PGSQLMPSKSPSTGTTEISSH |
| | | ESSHGTPSQTTAKNWELTASA |
| | | SHQPPGVYPQGHSDTTVAIST |
| | | STVLLCGLSAVSLLACYLKSR |
| | | QTPPLASVEMEAMEALPVTWG |
| | | TSSRDEDLENCSHHL |
| 7 | Human | MAPRRARGCRTLGLPALLLLL |
| | soluble | LLRPPATRGITCPPPMSVEHA |
| | IL-15Rα | DIWVKSYSLYSRERYICNSGF |
| | with | KRKAGTSSLTECVLNKATNVA |
| | signal | HWTTPSLKCIRDPALVHQRPA |
| | peptide | PPSTVTTAGVTPQPESLSPSG |
| | | KEPAASSPSSNNTAATTAAIV |
| | | PGSQLMPSKSPSTGTTEISSH |
| | | ESSHGTPSQTTAKNWELTASA |
| | | SHQPPGVYPQG |
| 8 | Human IL- | atggccccgc ggcgggcgcg |
| | 15Rα | cggctgccgg accctcggtc |
| | with | tcccggcgct gctactgctg |
| | signal | ctgctgctcc ggccgccggc |
| | peptide | gacgcggggc atcacgtgcc |
| | DNA | ctccccccat gtccgtggaa |
| | | cacgcagaca tctgggtcaa |
| | | gagctacagc ttgtactcca |
| | | gggagcggta catttgtaac |
| | | tctggtttca agcgtaaagc |
| | | cggcacgtcc agcctgacgg |
| | | agtgcgtgtt gaacaaggcc |
| | | acgaatgtcg cccactggac |
| | | aacccccagt ctcaaatgca |
| | | ttagagaccc tgccctggtt |
| | | caccaaaggc cagcgccacc |
| | | ctccacagta acgacggcag |
| | | gggtgacccc acagccagag |
| | | agcctctccc cttctggaaa |
| | | agagcccgca gcttcatctc |
| | | ccagctcaaa caacacagcg |
| | | gccacaacag cagctattgt |
| | | cccgggctcc cagctgatgc |
| | | cttcaaaatc accttccaca |
| | | ggaaccacag agataagcag |
| | | tcatgagtcc tcccacggca |
| | | ccccctctca gacaacagcc |
| | | aagaactggg aactcacagc |
| | | atccgcctcc caccagccgc |
| | | caggtgtgta tccacagggc |
| | | cacagcgaca ccactgtggc |
| | | tatctccacg tccactgtcc |
| | | tgctgtgtgg gctgagcgct |
| | | gtgtctctcc tggcatgcta |
| | | cctcaagtca aggcaaactc |
| | | ccccgctggc cagcgttgaa |
| | | atggaagcca tggaggctct |
| | | gccggtgact tgggggacca |
| | | gcagcagaga tgaagacttg |
| | | gaaaactgct ctcaccacct |
| | | atga |
| 9 | Human soluble | atggccccgc ggcgggcgcg |
| | IL-15Rα | cggctgccgg accctcggtc |
| | with | tcccggcgct gctactgctg |
| | signal | ctgctgctcc ggccgccggc |
| | peptide | gacgcggggc atcacgtgcc |
| | DNA | ctccccccat gtccgtggaa |
| | | cacgcagaca tctgggtcaa |
| | | gagctacagc ttgtactcca |
| | | gggagcggta catttgtaac |
| | | tctggtttca agcgtaaagc |
| | | cggcacgtcc agcctgacgg |
| | | agtgcgtgtt gaacaaggcc |
| | | acgaatgtcg cccactggac |
| | | aacccccagt ctcaaatgca |
| | | ttagagaccc tgccctggtt |
| | | caccaaaggc cagcgccacc |
| | | ctccacagta acgacggcag |
| | | gggtgacccc acagccagag |
| | | agcctctccc cttctggaaa |
| | | agagcccgca gcttcatctc |
| | | ccagctcaaa caacacagcg |
| | | gccacaacag cagctattgt |
| | | cccgggctcc cagctgatgc |
| | | cttcaaaatc accttccaca |
| | | ggaaccacag agataagcag |
| | | tcatgagtcc tcccacggca |
| | | ccccctctca gacaacagcc |
| | | aagaactggg aactcacagc |
| | | atccgcctcc caccagccgc |
| | | caggtgtgta tccacagggc |
| 10 | Human | ITCPPPMSVEHADIWVKSYSL |
| | soluble | YSRERYICNSGFKRKAGTSSL |
| | IL-15Rα | TECVLNKATNVAHWTTPSLKC |
| | (PQG | IRDPALVHQRPAPPSTVTTAG |
| | termination) | VTPQPESLSPSGKEPAASSPS |
| | | SNNTAATTAAIVPGSQLMPSK |
| | | SPSTGTTEISSHESSHGTPSQ |
| | | TTAKNWELTASASHQPPGVYP |
| | | QG |
| 11 | IL-15Ra codon | cctggccatt gcatacgttg |
| | optimized | tatccatatc ataatatgta |
| | DNA | catttatatt ggctcatgtc |
| | | caacattacc gccatgttga |
| | | cattgattat tgactagtta |
| | | ttaatagtaa tcaattacgg |
| | | ggtcattagt tcatagccca |
| | | tatatggagt tccgcgttac |
| | | ataacttacg gtaaatggcc |
| | | cgcctggctg accgcccaac |
| | | gacccccgcc cattgacgtc |
| | | aataatgacg tatgttccca |
| | | tagtaacgcc aatagggact |
| | | ttccattgac gtcaatgggt |
| | | ggagtattta cggtaaactg |
| | | cccacttggc agtacatcaa |
| | | gtgtatcata tgccaagtac |
| | | gccccctatt gacgtcaatg |
| | | atggtaaatg gcccgcctgg |
| | | cattatgccc agtacatgac |
| | | cttatgggac tttcctactt |
| | | ggcagtacat ctacgtatta |
| | | gtcatcgcta ttaccatggt |
| | | gatgcggttt tggcagtaca |
| | | tcaatgggcg tggatagcgg |
| | | tttgactcac ggggatttcc |
| | | aagtctccac cccattgacg |
| | | tcaatgggag tttgttttgg |
| | | caccaaaatc aacgggactt |
| | | tccaaaatgt cgtaacaact |
| | | ccgccccatt gacgcaaatg |
| | | ggcggtaggc gtgtacggtg |
| | | ggaggtctat ataagcagag |
| | | ctcgtttagt gaaccgtcag |
| | | atcgcctgga gacgccatcc |
| | | acgctgtttt gacctccata |
| | | gaagacaccg ggaccgatcc |
| | | agcctccgcg ggcgcgcgtc |
| | | gacgctagca agaaatggcc |
| | | ccgaggcggg cgcgaggctg |
| | | ccggaccctc ggtctcccgg |
| | | cgctgctact gctcctgctg |
| | | ctccggccgc cggcgacgcg |
| | | gggcatcacg tgcccgcccc |
| | | ccatgtccgt ggagcacgca |
| | | gacatctggg tcaagagcta |
| | | cagcttgtac tcccgggagc |
| | | ggtacatctg caactcgggt |
| | | ttcaagcgga aggccggcac |
| | | gtccagcctg acggagtgcg |
| | | tgttgaacaa ggccacgaat |
| | | gtcgcccact ggacgacccc |
| | | ctcgctcaag tgcatccgcg |
| | | acccggccct ggttcaccag |
| | | cggcccgcgc caccctccac |
| | | cgtaacgacg gcgggggtga |
| | | ccccgcagcc ggagagcctc |
| | | tccccgtcgg gaaaggagcc |
| | | cgccgcgtcg tcgcccagct |
| | | cgaacaacac ggcggccaca |
| | | actgcagcga tcgtcccggg |
| | | ctcccagctg atgccgtcga |
| | | agtcgccgtc cacgggaacc |
| | | acggagatca gcagtcatga |
| | | gtcctcccac ggcaccccct |
| | | cgcaaacgac ggccaagaac |
| | | tgggaactca cggcgtccgc |
| | | ctcccaccag ccgccggggg |
| | | tgtatccgca aggccacagc |
| | | gacaccacgg tggcgatctc |
| | | cacgtccacg gtcctgctgt |
| | | gtgggctgag cgcggtgtcg |
| | | ctcctggcgt gctacctcaa |
| | | gtcgaggcag actcccccgc |
| | | tggccagcgt tgagatggag |
| | | gccatggagg ctctgccggt |
| | | gacgtggggg accagcagca |
| | | gggatgagga cttggagaac |
| | | tgctcgcacc acctataatg |
| | | agaattcgat ccagatctgc |
| | | tgtgccttct agttgccagc |
| | | catctgttgt ttgcccctcc |
| | | cccgtgcctt ccttgaccct |
| | | ggaaggtgcc actcccactg |
| | | tcctttccta ataaaatgag |
| | | gaaattgcat cgcattgtct |
| | | gagtaggtgt cattctattc |
| | | tggggggtgg ggtggggcag |
| | | gacagcaagg gggaggattg |
| | | ggaagacaat agcaggcatg |
| | | ctggggatgc ggtgggctct |
| | | atgggtaccc aggtgctgaa |
| | | gaattgaccc ggttcctcct |
| | | gggccagaaa gaagcaggca |
| | | catccccttc tctgtgacac |
| | | accctgtcca cgcccctggt |
| | | tcttagttcc agccccactc |
| | | ataggacact catagctcag |
| | | gagggctccg ccttcaatcc |
| | | cacccgctaa agtacttgga |
| | | gcggtctctc cctccctcat |
| | | cagcccacca aaccaaacct |
| | | agcctccaag agtgggaaga |
| | | aattaaagca agataggcta |
| | | ttaagtgcag agggagagaa |
| | | aatgcctcca acatgtgagg |
| | | aagtaatgag agaaatcata |
| 12 | IL-15Ra codon | MAPRRARGCRTLGLPALLLLL |
| | optimized | LLRPPATRGITCPPPMSVEHA |
| | amino acid | DIWVKSYSLYSRERYICNSGF |
| | | KRKAGTSSLTECVLNKATNVA |
| | | HWTTPSLKCIRDPALVHQRPA |
| | | PPSTVTTAGVTPQPESLSPSG |
| | | KEPAASSPSSNNTAATTAAIV |
| | | PGSQLMPSKSPSTGTTEISSH |
| | | ESSHGTPSQTTAKNWELTASA |
| | | SHQPPGVYPQGHSDTTVAIST |
| | | STVLLCGLSAVSLLACYLKSR |
| | | QTPPLASVEMEAMEALPVTWG |
| | | TSSRDEDLENCSHHL |
| 13 | CMV IL-15Ra | cctggccatt gcatacgttg |
| | codon | tatccatatc ataatatgta |
| | optimized | catttatatt ggctcatgtc |
| | DNA | caacattacc gccatgttga |
| | | cattgattat tgactagtta |
| | | ttaatagtaa tcaattacgg |
| | | ggtcattagt tcatagccca |
| | | tatatggagt tccgcgttac |
| | | ataacttacg gtaaatggcc |
| | | cgcctggctg accgcccaac |
| | | gacccccgcc cattgacgtc |
| | | aataatgacg tatgttccca |
| | | tagtaacgcc aatagggact |
| | | ttccattgac gtcaatgggt |
| | | ggagtattta cggtaaactg |
| | | cccacttggc agtacatcaa |
| | | gtgtatcata tgccaagtac |
| | | gccccctatt gacgtcaatg |
| | | atggtaaatg gcccgcctgg |
| | | cattatgccc agtacatgac |
| | | cttatgggac tttcctactt |
| | | ggcagtacat ctacgtatta |
| | | gtcatcgcta ttaccatggt |
| | | gatgcggttt tggcagtaca |
| | | tcaatgggcg tggatagcgg |
| | | tttgactcac ggggatttcc |
| | | aagtctccac cccattgacg |
| | | tcaatgggag tttgttttgg |
| | | caccaaaatc aacgggactt |
| | | tccaaaatgt cgtaacaact |
| | | ccgccccatt gacgcaaatg |
| | | ggcggtaggc gtgtacggtg |
| | | ggaggtctat ataagcagag |
| | | ctcgtttagt gaaccgtcag |
| | | atcgcctgga gacgccatcc |
| | | acgctgtttt gacctccata |
| | | gaagacaccg ggaccgatcc |
| | | agcctccgcg ggcgcgcgtc |
| | | gacgctagca agaaatggcc |
| | | ccgaggcggg cgcgaggctg |
| | | ccggaccctc ggtctcccgg |
| | | cgctgctact gctcctgctg |
| | | ctccggccgc cggcgacgcg |
| | | gggcatcacg tgcccgcccc |
| | | ccatgtccgt ggagcacgca |
| | | gacatctggg tcaagagcta |
| | | cagcttgtac tcccgggagc |
| | | ggtacatctg caactcgggt |
| | | ttcaagcgga aggccggcac |
| | | gtccagcctg acggagtgcg |
| | | tgttgaacaa ggccacgaat |
| | | gtcgcccact ggacgacccc |
| | | ctcgctcaag tgcatccgcg |
| | | acccggccct ggttcaccag |
| | | cggcccgcgc caccctccac |
| | | cgtaacgacg gcgggggtga |
| | | ccccgcagcc ggagagcctc |
| | | tccccgtcgg gaaaggagcc |
| | | cgccgcgtcg tcgcccagct |
| | | cgaacaacac ggcggccaca |
| | | actgcagcga tcgtcccggg |
| | | ctcccagctg atgccgtcga |
| | | agtcgccgtc cacgggaacc |
| | | acggagatca gcagtcatga |
| | | gtcctcccac ggcaccccct |
| | | cgcaaacgac ggccaagaac |
| | | tgggaactca cggcgtccgc |
| | | ctcccaccag ccgccggggg |
| | | tgtatccgca aggccacagc |
| | | gacaccacgt aatgagaatt |
| | | cgcggatatc ggttaacgga |
| | | tccagatctg ctgtgccttc |
| | | tagttgccag ccatctgttg |
| | | tttgcccctc ccccgtgcct |
| | | tccttgaccc tggaaggtgc |
| | | cactcccact gtcctttcct |
| | | aataaaatga ggaaattgca |
| | | tcgcattgtc tgagtaggtg |
| | | tcattctatt ctggggggtg |
| | | gggtggggca ggacagcaag |
| | | ggggaggatt gggaagacaa |
| | | tagcaggcat gctggggatg |
| | | cggtgggctc tatgggtacc |
| | | caggtgctga agaattgacc |
| | | cggttcctcc tgggccagaa |
| | | agaagcaggc acatcccctt |
| | | ctctgtgaca caccctgtcc |
| | | acgcccctgg ttcttagttc |
| | | cagccccact cataggacac |
| | | tcatagctca ggagggctcc |
| | | gccttcaatc ccacccgcta |
| | | aagtacttgg agcggtctct |
| | | ccctccctca tcagcccacc |
| | | aaaccaaacc tagcctccaa |
| | | gagtgggaag aaattaaagc |
| | | aagataggct attaagtgca |
| | | gagggagaga aaatgcctcc |
| | | aacatgtgag gaagtaatga |
| | | gagaaatcat a |
| 14 | CMV IL-15Rα | MAPRRARGCRTLGLPALLLLL |
| | codon | LLRPPATRG1TCPPPMSVEHA |
| | optimized | DIWVKSYSLYSRERYICNSGF |
| | amino acid | KRKAGTSSLTECVLNKATNVA |
| | | HWTTPSLKCIRDPALVHQRPA |
| | | PPSTVTTAGVTPOPESLSPSG |
| | | KEPAASSPSSNNTAATTAAIV |
| | | PGSQLMPSKSPSTGTTEISSH |
| | | ESSHGTPSQTTAKNWELTASA |
| | | SHQPPGVYPQGHSDTT |
| 15 | C-terminal | PQGHSDTT |
| | of | |
| | soluble | |
| | human | |
| | IL-15Rα | |
| 16 | C-terminal | PQGHSDT |
| | of | |
| | soluble | |
| | human | |
| | IL-15Rα | |
| 17 | C-terminal | PQGHSD |
| | of | |
| | soluble | |
| | human | |
| | IL-15Rα | |
| 18 | C-terminal of | PQGHS |
| | soluble human | |
| | IL-15Rα | |
| 19 | C-terminal of | PQGH |
| | soluble human | |
| | IL-15Rα | |
| 20 | C-terminal of | PQG |
| | soluble human | |
| | IL-15Rα | |
| 21 | Human soluble | ITCPPPMSVEHADIWVKSYSL |
| | IL-15Rα | YSRERYICNSGFKRKAGTSSL |
| | | TECVLNKATNVAHWTTPSLKC |
| | | IRDPALVHQRPAPPSTVTTAG |
| | | VTPQPESLSPSGKEPAASSPS |
| | | SNNTAATTAAIVPGSQLMPSK |
| | | SPSTGTTEISSHESSHGTPSQ |
| | | TTAKNWELTASASHQPPGVYP |
| | | QGHSDTT |
| 22 | IL-15Rα | NWELTASASHQPPGVYPQG |
| | O- | |
| | glycosylation | |
| 23 | IL-15Rα | ITCPPPMSVEHADIWVK |
| | N- | |
| | glycosylation | |
| 24 | IL-15Rα | ITCPPPMSVEHADIWVKSYSL |
| | N- | YSRERYICNS |
| | glycosylation | |
| 25 | IL-15Rα | RXXR |
| | heterologous | |
| | protease | |
| | cleavage | |
| | site | |
| | recognized | |
| | by | |
| | furin | |
| | protease | |
| | Xaa = any | |
| | amino acid | |
| 26 | IL-15Rα | XXPRXX |
| | heterologous | |
| | protease | |
| | cleavage | |
| | site | |
| | 1,2 Xaa = | |
| | hydrophobic | |
| | amino acids | |
| | 5,6 | |
| | Xaa = non- | |
| | acidic | |
| | amino | |
| | acids | |
| 27 | Synthetic | MAPRRARGCRTLGLPALLLLLL |
| | sIL- | LRPPATRGITCPPPMSVEHADI |
| | 15Rαlpha- | WVKSYSLYSRERYICNSGFKRK |
| | Fc | AGTSSLTECVLNKATNVAHWTT |
| | fusion | PSLKCIRDPALVHQRPAPPSTV |
| | protein | TTAGVTPQPESLSPSGKEPAAS |
| | huIL15sRa205- | SPSSNNTAATTAAIVPGSQLMP |
| | Fc | SKSPSTGTTEISSHESSHGTPS |
| | | QTTAKNWELTASASHQPPGVYP |
| | | QGHSDTTPKSCDKTHTCPPCPA |
| | | PELLGGPSVFLFPPKPKDTLMI |
| | | SRTPEVTCVVVDVSHEDPEVKF |
| | | N |
| 18 | C-terminal of | PQGHS |
| | soluble human | |
| | IL-15Rα | |
| 19 | C-terminal of | PQGH |
| | soluble human | |
| | IL-15Rα | |
| 20 | C-terminal of | PQG |
| | soluble human | |
| | IL-15Rα | |
| 21 | Human soluble | ITCPPPMSVEHADIWVKSYSL |
| | IL-15Rα | YSRERYICNSGFKRKAGTSSL |
| | | TECVLNKATNVAHWTTPSLKC |
| | | IRDPALVHQRPAPPSTVTTAG |
| | | VTPQPESLSPSGKEPAASSPS |
| | | SNNTAATTAAIVPGSQLMPSK |
| | | SPSTGTTEISSHESSHGTPSQ |
| | | TTAKNWELTASASHQPPGVYP |
| | | QGHSDTT |
| 22 | IL-15Rα | NWELTASASHQPPGVYPQG |
| | O- | |
| | glycosylation | |
| 23 | IL-15Rα | ITCPPPMSVEHADIWVK |
| | N- | |
| | glycosylation | |
| 24 | IL-15Rα | ITCPPPMSVEHADIWVKSYSL |
| | N- | YSRERYICNS |
| | glycosylation | |
| 25 | IL-15Rα | RXXR |
| | heterologous | |
| | protease | |
| | cleavage | |
| | site | |
| | recognized | |
| | by | |
| | furin | |
| | protease | |
| | Xaa = any | |
| | amino acid | |
| 26 | IL-15Rct | XXPRXX |
| | heterologous | |
| | protease | |
| | cleavage | |
| | site | |
| | 1,2 Xaa = | |
| | hydrophobic | |
| | amino acids | |
| | 5,6 | |
| | Xaa = non- | |
| | acidic | |
| | amino | |
| | acids | |
| 27 | Synthetic | MAPRRARGCRTLGLPALLLLLL |
| | sIL- | LRPPATRGITCPPPMSVEHADI |
| | 15Rαlpha- | WVKSYSLYSRERYICNSGFKRK |
| | Fc | AGTSSLTECVLNKATNVAHWTT |
| | fusion | PSLKCIRDPALVHQRPAPPSTV |
| | protein | TTAGVTPQPESLSPSGKEPAAS |
| | huIL15sRa205- | SPSSNNTAATTAAIVPGSQLMP |
| | Fc | SKSPSTGTTEISSHESSHGTPS |
| | | QTTAKNWELTASASHQPPGVYP |
| | | QGHSDTTPKSCDKTHTCPPCPA |
| | | PELLGGPSVFLFPPKPKDTLMI |
| | | SRTPEVTCVVVDVSHEDPEVKF |
| | | NWYVDGVEVHNAKTKPREEQYN |
| | | STYRWSVLTVLHQDWLNGKEY |
| | | KCKVSNKALPAPIEKTISKAK |
| | | GQPREPQVYTLPPSRDELTKN |
| | | QVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| 28 | synthetic | MAPRRARGCRTLGLPALLLLL |
| | sIL- | LLRPPATRGITCPPPMSVEHA |
| | 15Rαlpha-Fc | DIWVKSYSLYSRERYICNSGF |
| | fusion | KRKAGTSSLTECVLNKATNVA |
| | protein | HWTTPSLKCIRDPALVHQRPA |
| | huIL15sRa200- | PPSTVTTAGVTPQPESLSPSG |
| | Fc | KEPAASSPSSNNTAATTAAIV |
| | | PGSQLMPSKSPSTGTTEISSH |
| | | ESSHGTPSQTTAKNWELTASA |
| | | SHQPPGVYPQGPKSCDKTHTC |
| | | PPCPAPELLGGPSVFLFPPKP |
| | | KDTLMISRTPEVTCVVVDVSH |
| | | EDPEVKFNWYVDGVEVHNAKT |
| | | KPREEQYNSTYRVVSVLTVLH |
| | | QDWLNGKEYKCKVSNKALPAP |
| | | IEKTISKAKGQPREPQVYTLP |
| | | PSRDELTKNQVSLTCLVKGFY |
| | | PSDIAVEWESNGQPENNYKTT |
| | | PPVLDSDGSFFLYSKLTVDKS |
| | | RWQQGNVFSCSVMHEALHNHY |
| | | TQKSLSLSPGK |

### EXAMPLES

### Example 1: Generation of hetIL-15 in CHO Cell Line

The Chinese hamster ovary (CHO) parental cell line CHO-MaKo was used to produce the IL-15/IL-15Rα heterodimer (also referred to as “hetIL-15”). CHO-MaKo cell line was derived by targeted deletion of the matriptase gene in CHO-C8TD using zinc finger nucleases (ZFNs) technique. The protease matriptase was found to be involved in the degradation of a variety of recombinant therapeutic proteins in CHO cells. CHO-C8TD was derived from a single vial of parental cell line CHO-K1PD from WCB070625. CHO-K1PD was derived from the CHO-K1 cell line, originally obtained from ATCC (cat. no. CCL-61.3). Details of the CHO-MaKo cell line can be found in WO2015/166427, which is incorporated herein by reference.

CHO-MaKo cells were cotransfected by electroporation with linearized vector pBW1697 (encoding IL-15 (interleukin 15) and IL015Rα (interleukin 15 receptor alpha)) and pBW1703 encoding IL-15Rα. After a recovery phase of two days, the transfected cell pool was cultivated in low folate medium supplemented with methotrexate (MTX) for several weeks to select for recombinant cells. To increase IL-15 expression, recovered cell pools were transfected by electroporation with linearized vector pBW1916 encoding IL-15. After a recovery phase of two days, the transfected cell pool was cultivated in low folate medium supplemented with methotrexate (MTX) and puromycin for several weeks to select for recombinant cells from which single cells were sorted by FACS. Selected clones were further characterized with regards to bioreactor performance, mRNA size and integrity (by Northern blot), transgene copy number (by qPCR), size and integrity of the expression cassettes (by Southern blot) and sequence verification (by NGS).

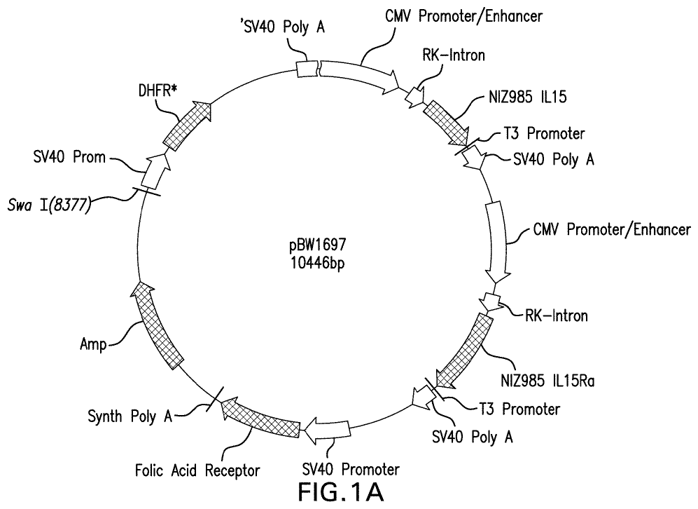

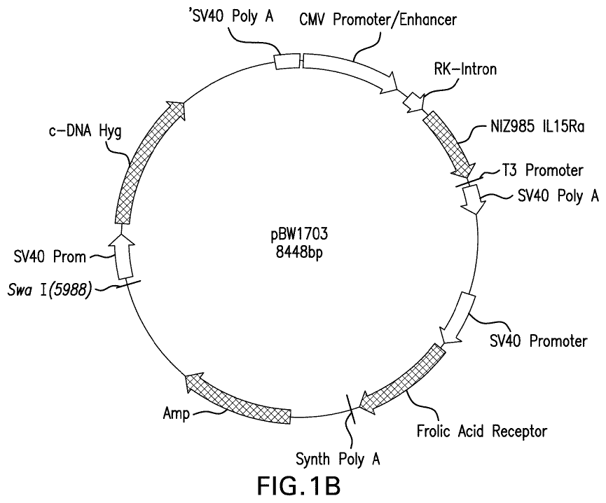

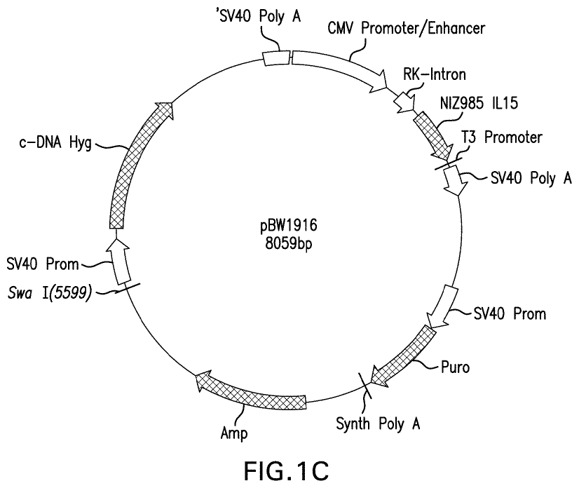

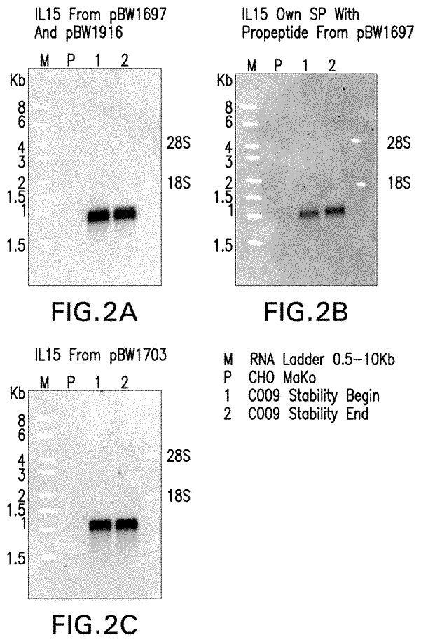

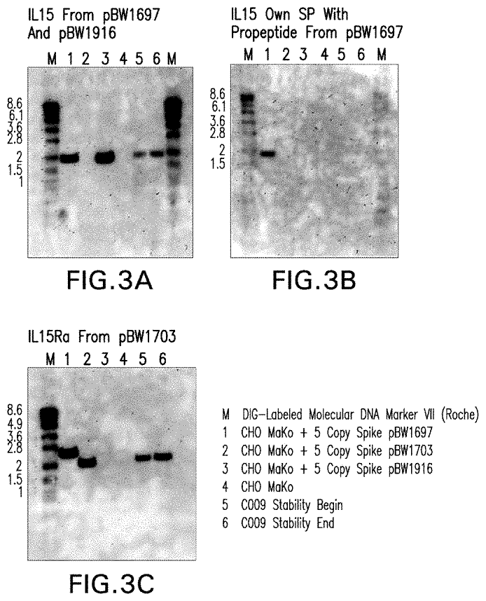

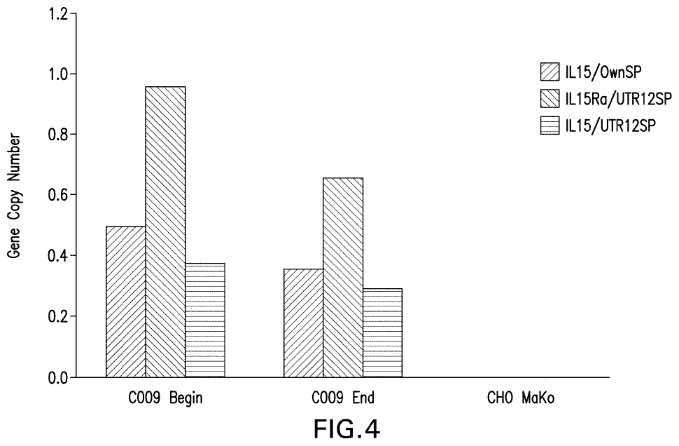

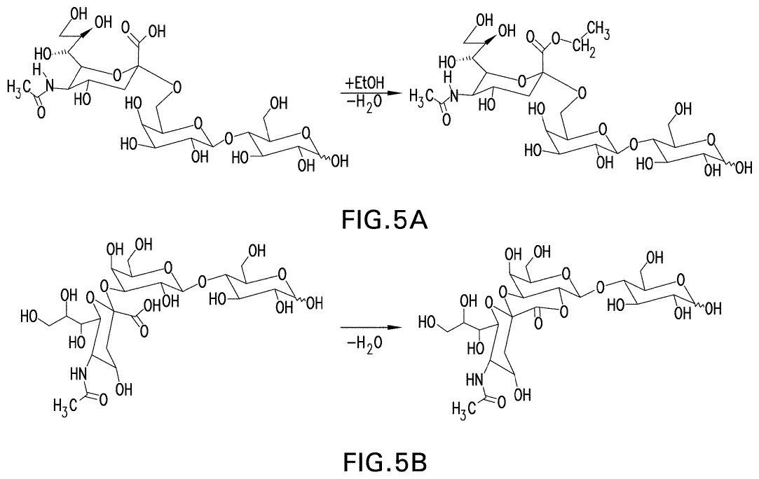

Vector maps of pBW1697, pBW1703 and pBW1916 are presented in FIG. 1. Table 3 provides the overview of final expression constructs and expression cassettes.

As shown in Table 1, two different IL-15 expression cassettes were used: in pBW1697, the native signal peptide (own SP) of 29 aa and the native propeptide sequence of 19 aa preceding the 114 aa IL-15 chain are included in the open reading frame (ORF). In pBW1916, the IL-15 chain is combined with the so-called UTR12 signal peptide (UTR12 SP).

IL-15Rα was also expressed from two different ORFs. In pBW1697, the full length receptor (IL-15Rα FL) with its' native signal peptide (own SP) is used. In pBW1703, a soluble version of IL-15Rα is expressed with the UTR12 SP.

TABLE 3
Overview of final expression constructs

| | IL-15 expression | IL-15Rα expression |
|---|---|---|
| Vector | cassette (ORF) | cassette (ORF) |
| pBW1697 | own SP + pro- | own SP + IL-15-Ra FL |
| | peptide + IL-15 | |
| pBW1703 | n.a | UTR12 SP + IL-15Rα sol |
| pBW1916 | UTR12 SP + IL-15 | n.a |

ORF: open reading frame, SP: signal peptide, FL: full length, n.a: not applicable, sol: soluble

### Example 2. Production of IL-15/IL-15Rα Heterodimer

The IL-15/IL-15Rα is produced by a recombinant Chinese Hamster ovary (CHO) cell line. The production is carried out using a standard fed-batch production process in a bioreactor.

One frozen vial from the master cell bank is thawed and suspended in the expansion medium. A series of shake flask passages are performed to expand the volume of the inoculum. When the inoculum volume and viable cell density are high enough (viable cell density of approximately 4.8×10⁶ cells/mL; viability>90%) the inoculum is transferred to the first seed reactor.

The inoculum from the previous step is transferred to the first seed bioreactor containing expansion medium and is further cultured in batch mode. When the viable cell density is sufficient (viable cell density of approximately 5.4×10⁶ cells/mL) the culture is used to inoculate the second seed bioreactor.

The culture from the first seed bioreactor is transferred to the second seed bioreactor containing expansion medium and is further cultured in batch mode. When the viable cell density is sufficient (viable cell density of approximately 5.6×10⁶ cells/mL) the culture is used to inoculate the production bioreactor.

The production bioreactor is operated in fed-batch mode. The culture from step 3 is transferred to the production bioreactor, which contains production medium. Feeding with two feed solutions is carried out throughout the bioreactor run. Both feed additions are started at a viable cell density of approximately 2×10⁶ cells/mL. At a viable cell density of approximately 12.5×10⁶ cells/mL, the cultivation temperature is shifted from 36.5° C. to 33.0° C. Harvest is initiated when cell viability drops to ≤75% or approximately 14 days after the start of production. Each lot of bulk harvest is monitored for bioburden and adventitious agents.

The purification process includes two steps which are dedicated to virus inactivation/removal, namely low pH incubation and nanofiltration. At the end the product is concentrated and diafiltrated into the final buffer.

### Example 3. Determination of O-Glycans Composition

O-glycans were analyzed by Matrix Assisted Laser Desorption Ionization Mass Spectrometry (MALDI-MS). For this purpose O-linked glycans are chemically cleaved from the protein by reductive beta-elimination method and derivatized by permethylation prior to MS detection. Based on the MS data, identification and semi-quantitative results are generated. The identity and relative abundance of the main glycan species in all five batches are summarized in Table 4.

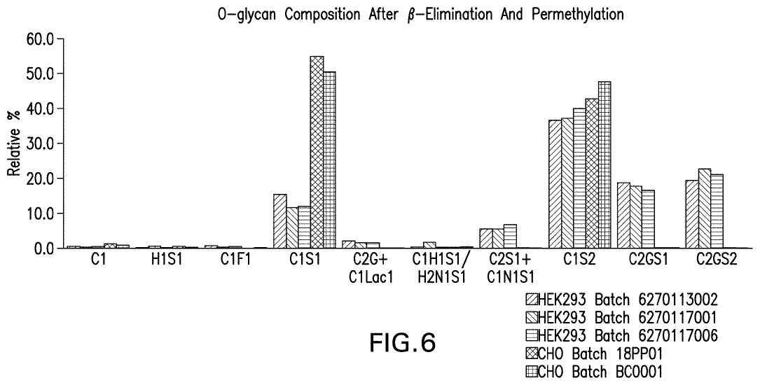

FIG. 6 visualizes the distribution of the various species in form of a bar-chart.

Relevant differences in O-glycan variants and distribution are observed between hetIL-15 batches derived from different cell lines. HEK293 batches contain approximately 50% of Core 2 type variants (C2G, C2S1, C2GS1, C2GS2). These are only detected at trace levels in CHO derived batches. A higher level of a Core 1 mono-sialylated variant (C1S1) was detected in CHO batches compared to HEK293 batches (˜50% vs ˜15%, respectively). The overall level of sialylation of all batches is very high (>97%). Due to the observed differences, batches derived from HEK293 and CHO cells are considered not comparable with respect to their general 0-glycan composition.

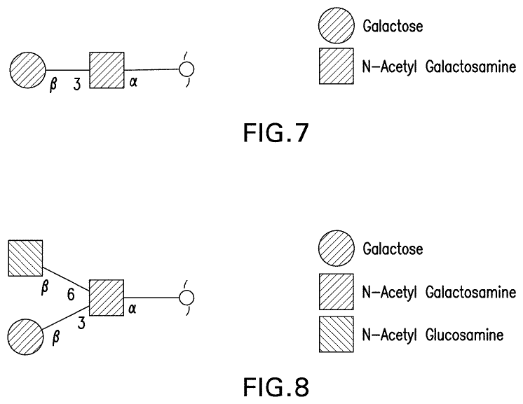

FIG. 7 shows the basic structure of Core 1 type O-glycan forms (“C1”). Extensions with other glycan residues (e.g Sialic acid) lead to more complex structures as listed in Table 4 (e.g C1S1, C1S2). The Core 1 structure motif is present in all analyzed batches. FIG. 8 below shows the basic structure of Core 2 type O-glycan forms (“C2”). Extensions with other glycan residues lead to more complex structures as listed in Table 4 (e.g C2S1, C2GS1). This structure has been detected at relevant levels only in HEK293 batches.

TABLE 4
O-glycan distribution

| | HEK293 | HEK293 | HEK293 | | |
|---|---|---|---|---|---|
| | batch | batch | batch | CHO batch | CHO batch |
| Glycan isoform | 6270113002 | 6270117001 | 6270117006 | 18PP01 | BC0001 |
| C1 | 0.57 | — | — | 1.2 | 0.92 |
| H1S1 | — | — | — | 0.51 | — |
| C1F1 | 0.71 | — | — | — | — |
| C1S1 | 15.3 | 11.7 | 12.0 | 54.9 | 50.5 |
| C2G + C1Lac1 | 2.1 | 1.6 | 1.6 | — | — |
| C1H1S1 or H2N1S1 | — | 1.7 | — | — | — |
| C2S1 + C1N1S1 | 5.5 | 5.6 | 6.8 | — | — |
| C1S2 | 36.6 | 37.2 | 40.0 | 42.8 | 47.6 |
| C2GS1 | 18.8 | 17.8 | 16.6 | — | — |
| C2GS2 | 19.4 | 22.7 | 21.1 | — | — |
| Sum of Fucosylated | 1.1 | 0.81 | 0.84 | — | — |
| species | | | | | |
| Sum of A-Sialylated | 3.5 | 2.4 | 2.6 | 1.2 | 0.93 |
| species | | | | | |
| Sum of Mono-Sialylated | 40.5 | 37.6 | 36.3 | 55.9 | 51.3 |
| species | | | | | |
| Sum of Di-Sialylated | 56.0 | 60.0 | 61.1 | 42.8 | 47.7 |
| species | | | | | |

“—“: not detected or below the reporting limit of 0.5%
“H”: Hexose
“F”: Fucose
“C”: Core
“Lac”: N-acethyl-lactosamine
“G”: Galactose (elongated core 2)
“S”: Sialic acid
“N”: N-acethylhexosamine

O-glycans were further characterized with respect to the linkage type of sialic acids. After chemical cleavage from the protein by the beta-elimination method, derivatization by ethylesterification was performed. The MS analysis of O-linked glycans after ethylesterification provides sialic acid linkage information (α2, 3 or α2, 6) and was performed on a qualitative basis only.

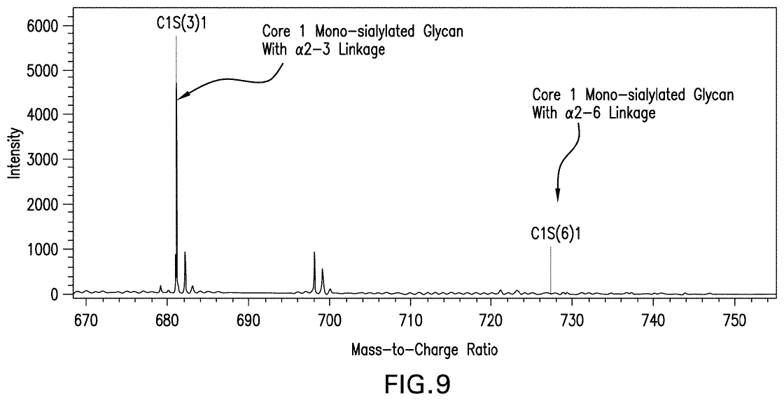

HEK293 and CHO batches both exhibit two types of sialic acid linkages (α2, 3 and α2, 6). This is expected for proteins expressed in human cells (HEK293) but uncommon in proteins expressed in CHO cells. FIG. 9 below shows a mass spectrum for CHO clinical batch BC0001. The relative intensity of α2, 6 linked sialic acid is rather low compared to the more dominant variant of α2, 3 linked sialic acid.

The sialic acid profile and content was evaluated by reverse phase chromatography. After chemical cleavage and fluorescent labelling, sialic acids were separated on the column by gradient elution and quantified by fluorescence detection.

N-acetylneuranimic acid is the predominant sialic acid in human glycoproteins. N-glycolyneuraminic acid is found in non-human glycoproteins and is undesired. Therefore a high ratio N-acetylneuranimic acid to N-glycolyneuraminic acid is preferred.

HEK293 batches exhibit very high ratio. The amount of N-glycolyneuraminic acid in theses batches can be considered negligible.

CHO batches exhibit high and comparable ratio of more than 200. This indicates that less than 0.5% of undesired N-glycolyneuraminic acid is present in these samples.

### Example 4. Determination of N-Glycans Composition

N-glycans were enzymatically cleaved from the hetIL-15 heterodimer and derivatized with a fluorescent label and a tertiary amine using the RapiFluor™ technology. Following purification, the labelled N-glycans were analyzed by HILIC (Hydrophilic Interaction Liquid Chromatography) with fluorescence detection coupled with MS (Mass Spectrometry).

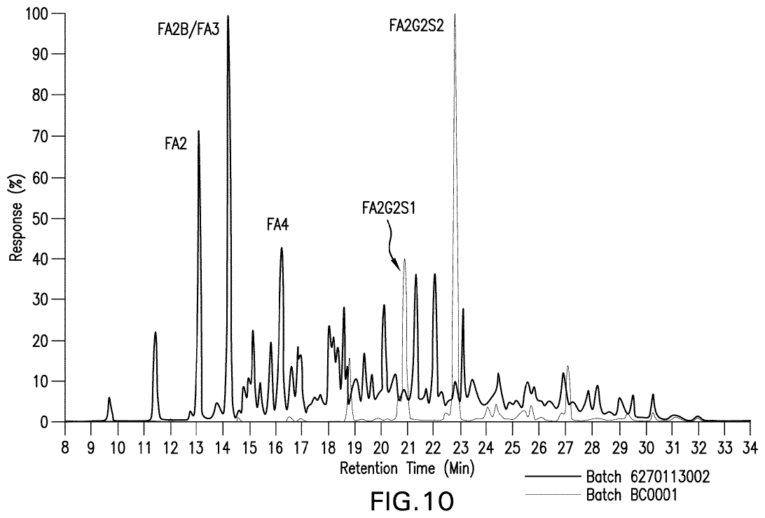

The chromatogram in FIG. 10 shows an overlay of one HEK293 derived batch and one CHO derived batch. Significant differences in N-glycan population and distribution are observed between CHO batches derived from the two different cell lines. Major N-glycan species are different and the profile of the HEK293 batch exhibits a heterogeneous distribution whereas the profile of the CHO batch is more homogeneous.

The identity and relative peak areas of the main N-glycan species in all five batches are summarized in Table 5.

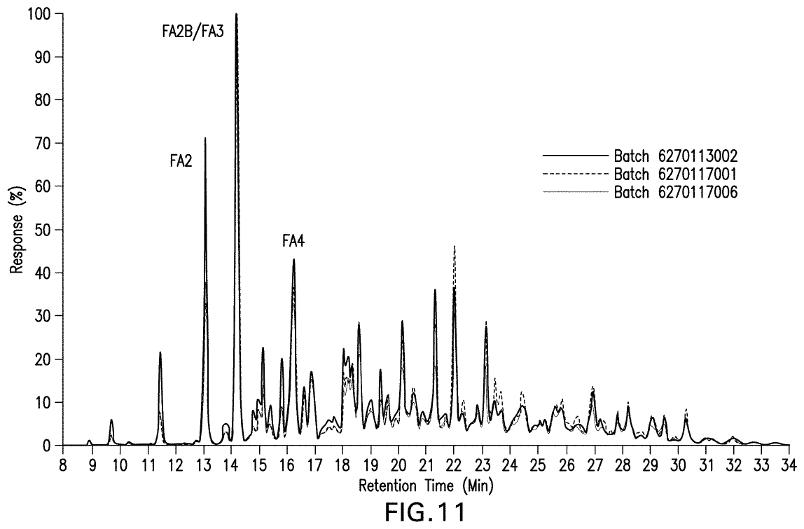

Chromatogram of FIG. 11 shows an overlay of the three HEK293 batches. The overall carbohydrate pattern of the three batches and the rank order of the major forms are similar and the observed differences are within the expected batch to batch variability. Whilst major species are related to galactosylation (FA2B/FA2/FA3/FA4), a high level of sialylation was observed. A high number of diverse sialylated glycans was detected at low levels. Species with a potential impact on pharmacokinetics or immunogenicity such as afucosylated and high mannose glycans were not detected.

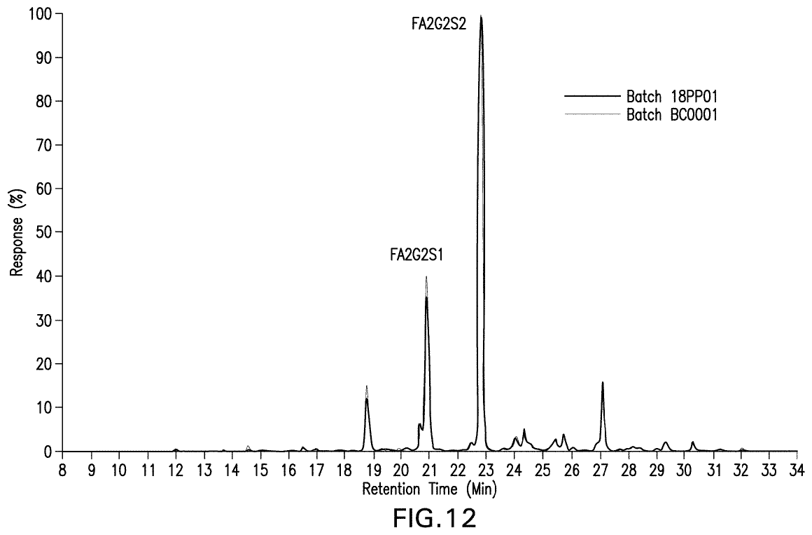

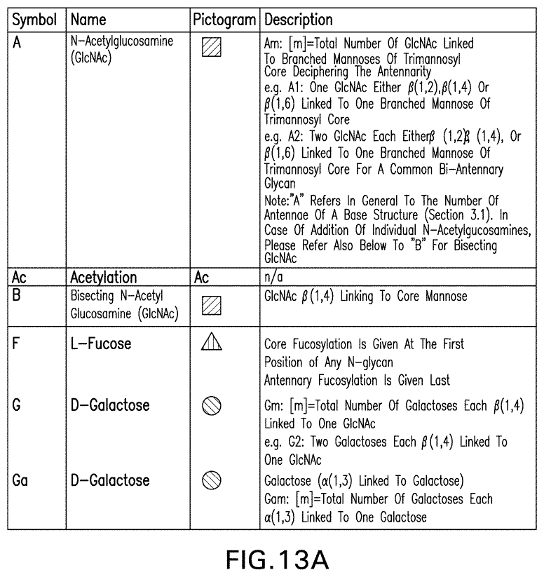

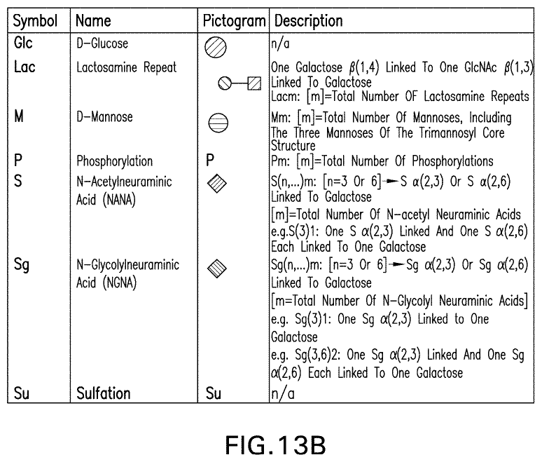

Chromatogram of FIG. 12 shows an overlay of the two CHO batches. The carbohydrate pattern of the two batches are very similar. Two major sialylated species (FA2G2S2 & FA2G2S1) contribute to approximately 60% of the N-glycan population. Species with a potential impact on pharmacokinetics or immunogenicity such as afucosylated and high mannose glycans were not detected.

TABLE 5
N-glycan distribution

| | HEK293 | HEK293 | HEK293 | | |
|---|---|---|---|---|---|
| | batch | batch | batch | CHO batch | CHO batch |
| N-glycan species | 6270113002 | 6270117001 | 6270117006 | 18PP01 | BC0001 |
| FM3 | 0.63 | — | — | — | — |
| FA1 species | 2.2 | 0.6 | [symbol] | — | — |
| FA2 | 7.4 | 3.7 | 4.0 | — | — |
| FA1G1 | — | — | — | — | 0.57 |
| FA26 and FA3 | 12.6 | 12.0 | 14.6 | — | — |
| FA4 and FA38 | 3.6 | 4.8 | 4.2 | — | — |
| FA2[6]G1 | 1.1 | 0.63 | 0.72 | — | — |
| FA2[3]G1 | 1.8 | 0.92 | 1.0 | — | 0.53 |
| FA1G1S1 species | — | — | — | — | 0.67 |
| FM6A1G1 | 0.59 | 0.55 | 0.87 | — | — |
| FA28G1 and FA361 | 5.5 | 4.1 | 6.3 | — | — |
| FA46 | 1.7 | 0.92 | 1.4 | — | — |
| FA3F1 | 0.81 | — | — | — | — |
| FA4G1 species | 1.5 | 1.1 | 1.4 | — | — |
| FA3F1G1 species | 1.8 | 2.1 | 1.9 | — | — |
| FA2G3 | 3.0 | 1.7 | 1.3 | 6.0 | 7.4 |
| FA28S1 | 0.52 | 1.4 | 2.0 | — | — |
| FM6A1G1S1 | 0.56 | 0.72 | — | — | — |
| FA2BG2 and FA3G2 | 3.3 | 3.4 | 3.2 | — | — |
| FA4BG1 | 0.70 | 0.95 | 1.3 | — | — |
| FA2G1S1 species | 3.3 | 1.6 | 1.6 | — | — |
| FA3F1G2 | 1.8 | 2.8 | 2.8 | — | — |
| FA2F1G2 species | 1.0 | 0.87 | 0.54 | — | — |
| FA28G1S1 species | 3.0 | 1.3 | 1.5 | — | — |
| FA2G2S1 species | 5.6 | 6.2 | 4.9 | 38.5 | 21.1 |
| FA4G2 | — | 0.68 | 0.86 | — | — |
| FA2BG2S1 and FA3G2S1 | 6.1 | 7.0 | 5.8 | 0.56 | — |
| FA2BF1G1S1 species | 0.75 | 1.7 | 1.2 | — | — |
| FA3F1G2S1 species | 0.61 | 2.8 | 1.8 | — | — |
| FA3G3 | 0.96 | 0.92 | 0.59 | 1.1 | 1.2 |
| FA4G3 species | 1.5 | 1.3 | 1.2 | — | — |
| FA4BG2S1 | — | — | 0.57 | — | — |
| FA4G3S1 species | 2.7 | 2.3 | 1.4 | — | — |
| FA2G2S2 species | 3.8 | 4.5 | 3.5 | 44.7 | 43.4 |
| FA3G2S2 species | 1.8 | 2.6 | 2.4 | — | — |
| FA4G4 | 0.53 | — | — | — | — |
| FA3G3S1 species | 1.1 | 2.0 | 0.67 | 2.7 | 2.7 |
| FA2F1G2S2 species | — | — | — | 3.7 | 3.8 |
| FA2F2G362 | — | — | — | 0.52 | — |
| FA4BG3S1 | 0.65 | — | 0.67 | — | — |
| FA3G3S2 species | 1.2 | 1.2 | 1.3 | 3.5 | 3.7 |
| FA4BF1G3S1 | — | — | 0.80 | — | — |
| FA3BG3S2 species | 0.90 | 1.2 | 0.99 | — | — |
| FA4BG4S1 | 0.96 | 1.8 | 1.5 | — | — |
| FA3G3S3 species | 0.91 | 0.3 | 1.7 | 8.0 | 7.8 |
| FA4BF1G4S1 species | — | 0.95 | — | — | — |
| FA4G4S1 speices | 0.85 | — | — | — | — |
| FA4G4S2 | 0.3 | 2.2 | 2.8 | 1.1 | 1.1 |
| FA4F1G4S2 species | 0.75 | — | — | — | — |
| FA4BS2G species | — | 1.9 | 1.4 | — | — |
| FA4BF1G4S2 species | — | 0.84 | 0.73 | — | — |
| FA4G4B3 species | 2.0 | 2.0 | 1.6 | 1.9 | 1.7 |
| FA4BG433 | — | 0.58 | — | — | — |
| FA4G4S4 species | 2.8 | 2.7 | 2.2 | 1.0 | 1.3 |
| FA4G4Loc1S4 species | — | 0.62 | 0.52 | — | — |
| Unknown peaks | — | — | — | 1.1 | 0.83 |

“—“: not detected or below the reporting limit of 0.5%
“F”: Fucose
“A”: Anternary N-acetyl glucosamine
“B”: Bisecting N-acetyl glucosamine
“M”: [symbol]
“Lac”: Lactosamine
“G”: Galactose
“S”: N-acetyl [symbol] [symbol]
indicates data missing or illegible when filed

### Example 5. Phase I/Ib Study of IL-15/IL-15Rα Complex Alone or in Combination with an Anti-PD-1 Antibody Molecule in Adults with Metastatic Cancer

This example describes a study to determine the safety, tolerability, dose-limiting toxicity (DLT) and maximum tolerated dose (MTD) of subcutaneous (SC) recombinant heterodimeric IL-15/soluble IL-15Rα complexes (hetIL-15) produced by CHO cell line, administered alone or in combination with the anti-PD-1 antibody molecule to human patients with solid tumors or lymphoma.

Objectives

The purpose of this phase I/Ib study is to determine the safety profile of heterodimeric IL-15/soluble IL-15Rα complexes (hetIL-15) produced by CHO cell line (referred to as “CHO hetIL-15”), and if it can be safely combined with anti-PD-1 antibody and to determine the appropriate dose and schedule for further study. Moreover, the study will characterize the pharmacokinetic profiles of CHO hetIL-15 as a single agent and in combination with anti-PD-1 antibody and identify preliminary anti-tumor activity.

Primary objective is to to characterize safety, tolerability of CHO hetIL-15 as a single agent and in combination with anti-PD-1 antibody in patients with solid tumors and lymphomas that previously responded to immune checkpoint inhibitor (CPI) and progressed (secondary resistant patients).

Secondary objectives are: 1) to assess preliminary anti-tumor activity of CHO hetIL-15 and anti-PD-1 antibody; and 2) to characterize the pharmacokinetics (PK) of CHO hetIL-15 as a single agent and in combination with anti-PD-1 antibody and PK of anti-PD-1 antibody.

Study Design

This is a phase I/Ib, open-label, global, multi-center study of subcutaneously administered CHO hetIL-15 alone and in combination with anti-PD-1 antibody in subjects with advanced solid tumors and lymphoma who have progressed after obtaining a previous response to anti-PD-1/CPI therapy. Previous response is defined as a radiographic complete response (CR) or a partial response (PR). Subjects with stable disease (SD) lasting ≥6 months will also be included if the most recent regimen included CPI. The study consists of two parts, dose escalation and dose expansion. Two separate arms will be examined during the escalation portion: 1) evaluation of CHO hetIL-15 as a single agent. Anti-PD-1 antibody may be added at the anti-PD-1 antibody of the first disease re-evaluation and 2) administration of CHO hetIL-15 and anti-PD-1 antibody as a combination starting from C1D1.

Patient Population

The study will be conducted in male and female patients≥18 years of age that were previously treated with CPI (anti PD-1/PD-L1 and/or anti CTLA 4) who have previously responded and progressed. Previous response is an initial radiographic CR/PR (a confirmatory scan is not required) or SD lasting ≥6 months if the most recent regimen included CPI. During dose escalation, the study will be conducted in patients with advanced solid tumors and lymphomas. During expansion the study will be conducted in patients with melanoma.

Key Inclusion Criteria

1. Male or female patients≥18 years of age

2. Histologically confirmed and documented advanced solid tumors and lymphoma (includes locally advanced that are not curable by surgery or radiotherapy, and those with metastatic disease) with documented progression following standard therapy, or for whom, in the opinion of the Investigator, no appropriate standard therapy exists.

Escalation: Patients previously treated with CPI (anti PD-1/PD-L1 and/or anti CTLA-4) who have previously responded and progressed. Previous response is an initial radiographic CR/PR (a confirmatory scan is not required) or SD lasting ≥6 months if the most recent regimen included CPI.

Expansion: Patients with melanoma previously treated with CPI (anti PD-1/PD-L1 and/or anti CTLA-4) who have previously responded and progressed. Previous response is radiographic CR/PR (a confirmatory scan is not required) or SD lasting ≥6 months if the most recent regimen included CPI.

Key Exclusion Criteria

1. Patients that have received any prior IL-15 treatment.

2. History of severe hypersensitivity reactions to any ingredient of study drug(s) and other mAbs and/or their excipients.

3. Patients with primary CNS tumors are excluded. Presence of symptomatic CNS metastases, or CNS metastases that require local CNS-directed therapy (such as radiotherapy or surgery), or increasing doses of corticosteroids 2 weeks prior to study entry. Patients with treated symptomatic brain metastases should be neurologically stable (for 4 weeks post-treatment and prior to study entry) and at a dose of ≤10 mg per day prednisone or equivalent for at least 2 weeks before administration of any study treatment.

4. Systemic chronic steroid therapy (>10 mg/day prednisone or equivalent) or any immunosuppressive therapy, other than replacement-dose steroids in the setting of adrenal insufficiency, within 7 days of the first dose of study treatment. Topical, inhaled, nasal and ophthalmic steroids are allowed.

5. Malignant disease, other than that being treated in this study. Exceptions include basal cell carcinoma of the skin or squamous cell carcinoma of the skin that has undergone potentially curative therapy or in situ cervical cancer or other tumors that will not affect life expectancy.

Efficacy Assessments

Tumor assessments as per RECIST1.1, iRECIST for solid tumors as described by Seymour et al. (2017) Lancet Oncol; 18:e143-e152 and Cheson et al (2014) J. Clin. Oncol. 32(27):3059-67 for lymphomas. At Screening, imaging assessments will be performed within 28 days of the start of treatment (Day −28 to Day −1 prior to Cycle 1 Day 1). All subjects will undergo a computed tomography (CT) scan with IV contrast of the chest, abdomen and pelvis. If there is clinical evidence of disease in the neck, a CT with IV contrast of the neck will also be performed. Imaging of the brain at baseline is required for subjects with known CNS disease. Magnetic Resonance Imaging (MRI) should be used to evaluate sites of disease that are not adequately imaged by CT. If a subject is intolerant of contrast agents, CTs may be performed without contrast. MRI may be used to evaluate sites of disease where a CT without IV contrast is not adequate. Visible skin lesions and easily palpable tumors may be measured by physical examination using a ruler or calipers and color photographs will be taken. Ultrasound should not be used to measure sites of disease for the purpose of response.

Outcome Measures

Pharmacokinetics are assessed by Serum concentration of CHO hetIL-15 and anti-PD-1 antibody at baseline and on treatment.

Incidence of Dose Limiting Toxicities (DLTs) in Cycle 1 (28 days) for CHO hetIL-15 as a single agent and in combination with anti-PD-1 antibody.

Incidence and severity of adverse events (AEs) and serious adverse events (SAEs), including changes in laboratory parameters, vital signs, and electrocardiograms (ECGs).

Other assessments include:

1. Assessment of changes in the numbers and cytotoxic activity of tumor infiltrating CD8+ T cells and NK cells and changes in the abundance of T cell clones.

2. Sequencing on baseline and on-treatment tumor biopsies. Evaluation of PD-L1 expression and CD8+ T cells on tumor biopsies by IHC.

3. Gene expression profiling on baseline and on-treatment tumor samples

4. DNA sequencing of circulating free tumor DNA from baseline and on-treatment plasma samples

5. Evaluation of changes from baseline in the level and/or expression of activation/proliferation markers, checkpoint inhibitors on T cell and NK cell subsets in blood and soluble immune factors in plasma

Study Treatment

Dose Escalation and Dose Expansion

Patients will be treated with CHO hetIL-15 as a single agent and in combination with anti-PD-1 antibody until the MTDs are reached or a lower RD is established for the combination. The dose escalation will be guided by an adaptive BHLRM following the EWOC principle to control the risk of dose-limiting toxicity (DLT) in future subjects on study. Two separate dosing arms will be evaluated during the dose escalation part:

Arm 1) CHO hetIL-15 single agent (subjects will be permitted to begin anti-PD-1 antibody after their first disease re-evaluation)

Arm 2) CHO hetIL-15 in combination with anti-PD-1 antibody starting at C1D1.

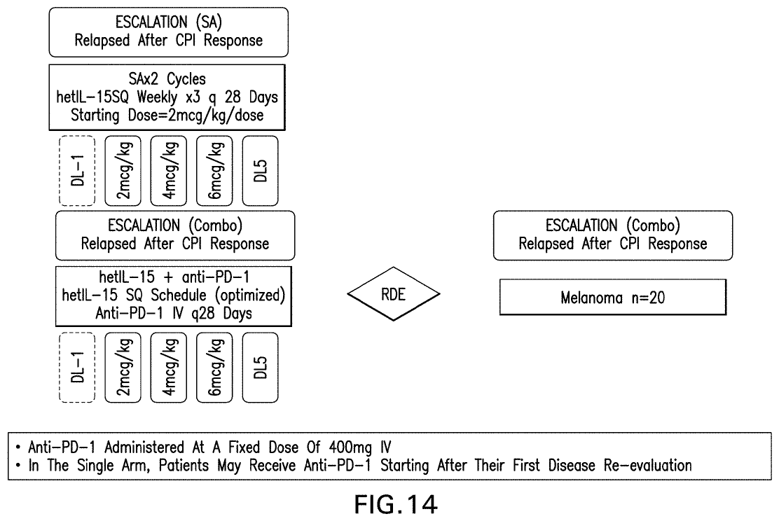

FIG. 14 provides the provisional dose levels that will be evaluated. It is possible for additional and/or intermediate dose levels to be added during the course of the study. In addition, alternate dosing schedules of hetIL-15 may be evaluated, for example, administering hetIL-15 once or twice weekly during the first two weeks of the cycle. Cohorts may be added at any dose level below the MTD in order to better understand safety, PK, and/or PD.

The treatment period will begin on Cycle 1 Day 1 (C1D1). Each treatment cycle will consist of 28 days. CHO hetIL-15 will be administered subcutaneously once a week, 3 weeks on/1 week off. When anti-PD-1 antibody is given, it will be administered intravenously at a fixed dose of 400 mg once on day 1 of each cycle.

The starting dose of CHO hetIL-15, alone or in combination with anti-PD-1 antibody, for subjects enrolled in this trial is 2 μg/kg subcutaneously (SC) once a week, on a 3 weeks on/1 week off schedule. The starting dose of anti-PD-1 antibody will be 400 mg Q4W i.v. Alternative dosing schedules may be explored the protocol will be amended to reflect any new schedule(s).

In pre-clinical models, IL-15 treatments alone induce IFN-γ and have anti-tumor effects but this anti-tumor activity may be limited due to upregulation of PD-1 on CD8+ T cells. Addition of checkpoint inhibition, such as PD-1 and CTLA-4 inhibitors, further increases IFN-γ expression and reduces expression of PD-1 on CD8+ T cells leading to increased survival (Yu et al (2010) Clin Cancer Res; 16(24):6019-28, Yu et al (2012) Proc Natl Acad Sci USA; 109(16):6187-92). Moreover, preliminary clinical data, have shown that subjects responding to CPIs have higher baseline levels of IL-15. CD8+ T-cell tumor infiltration in melanoma, NSCLC and breast cancer is also correlated with higher levels of IL-15 and NK gene signatures. Therefore, the combination of CHO hetIL-15, an IL-15 agonist, with anti-PD-1 antibody, a PD-1 inhibitor, may be synergistic and may further enhance anti-tumor responses, particularly in subject populations that have previously responded to checkpoint inhibition, and subsequently relapsed.

Additional agents may be combined with CHO hetIL-15±anti-PD-1 antibody and other indications may be considered in the expansion part of the study. These additional combination regimens and/or indications will only be explored if added to this trial with a future protocol amendment.

The dose expansion portion of the study will start once the MTD and/or RD is declared for CHO hetIL-15 in combination with anti-PD-1 antibody. The primary goal of the expansion group is to further evaluate the safety and tolerability of CHO hetIL-15 in combination with anti-PD-1 antibody. A secondary goal of the expansion group is to assess the anti-tumor activity of CHO hetIL-15 in combination with anti-PD-1 antibody in CPI relapsed melanoma subjects.

Provisional Dose Levels

Table 6 and Table 7 describes the starting dose and the provisional dose levels for CHO hetIL-15 that may be evaluated during this trial. The anti-PD-1 antibody dose is fixed at 400 mg Q4W. With the exception of starting dose level 1, actual dose levels will be determined based on available toxicity, pharmacokinetic and pharmacodynamic data.

TABLE 6
Provisional dose levels for CHO hetIL-15 single agent
(Arm 1) followed by a anti-PD-1 antibody combination

| | Proposed | Increment | | |
|---|---|---|---|---|
| Dose | weekly | from previous | Anti-PD-1 dose Q4W (if added | Increment from previous |
| level | dose* | hetIL-15 dose | after the first disease re-evaluation) | Anti-PD-1 dose*** |
| −1** | 1 μg/kg | −50% | 400 mg | 0% |
| 1 | 2 μg/kg | (starting dose) | 400 mg | (starting dose) |
| 2 | 4 μg/kg | 100% | 400 mg | 0% |
| 3 | 8 μg/kg | 100% | 400 mg | 0% |
| 4 | 16 μg/kg | 100% | 400 mg | 0% |

*It is possible for additional and/or intermediate dose levels to be added during the course of the study. Cohorts may be added at any dose level below the MTD in order to better understand safety, PK or PD. Multiple dose levels below the MTD may be evaluated simultaneously in order to obtain PK and PD data across a range of doses.
**Dose level −1 represent treatment doses for subjects requiring a dose reduction from the starting dose level.
***No dose reductions are allowed for anti-PD-1 antibody. Toxicity related to anti-PD-1 antibody will be managed by omission or delay.

TABLE 7
Provisional dose levels for CHO hetIL-15 and anti-PD-1 Combination (Arm 2)

| Dose | Proposed | Increment from previous | Anti-PD-1 | Increment from previous |
|---|---|---|---|---|
| level | weekly dose* | hetIL-15 dose | dose Q4W | Anti-PD-1 dose*** |
| −1** | 1 μg/kg | −50% | 400 mg | 0% |
| | | | (starting at C1D1) | |
| 1 | 2 μg/kg | (starting dose) | 400 mg | (starting dose) |
| | | | (starting at C1D1) | |
| 2 | 4 μg/kg | 100% | 400 mg | 0% |
| | | | (starting at C1D1) | |
| 3 | 8 μg/kg | 100% | 400 mg | 0% |
| | | | (starting at C1D1) | |
| 4 | 16 μg/kg | 100% | 400 mg | 0% |
| | | | (starting at C1D1) | |

*It is possible for additional and/or intermediate dose levels to be added during the course of the study. Cohorts may be added at any dose level below the MTD in order to better understand safety, PK or PD. Multiple dose levels below the MTD may be evaluated simultaneously in order to obtain PK and PD data across a range of doses.
**Dose level −1 represent treatment doses for subjects requiring a dose reduction from the starting dose level.
***No dose reductions are allowed for anti-PD-1 antibody. Toxicity related to anti-PD-1 antibody will be managed by omission or delay.

Definition of Dose-Limiting Toxicities

A dose-limiting toxicity (DLT) is defined as an adverse event or abnormal laboratory value that occurs within the DLT period (28 days) where the relationship to CHO hetIL-15 as a single agent or the CHO hetIL-15/anti-PD-1 combination, cannot be ruled out, and is not primarily related to disease, disease progression, inter-current illness, or concomitant medications. Table 8 lists criteria for DLTs. The National Cancer Institute Common Terminology Criteria for Adverse events (NCI CTCAE) version 5.0 will be used for all grading. For the purpose of dose-escalation decisions, DLTs will be considered and included in the BHLRM.

TABLE 8
Criteria for defining dose-limiting toxicities
During dose escalation, any Grade 3 or Grade 4 AEs related to study
treatment occurring during Cycle 1 are DLTs, EXCEPT:

| | |
|---|---|
| Fatigue | Grade 3 fatigue that resolves to ≤ Grade 1 within 7 days. |
| Hypertension | Grade 3 hypertension that resolves within 7 days after starting |
| | anti-hypertensive therapy. |
| Gastrointestinal | Grade 3 nausea and vomiting that resolves to ≤ Grade 1 within |
| | 48 hours of starting optimal anti-emetic therapy. |
| | Grade 3 diarrhea that resolves within 7 days after starting optimal |
| | anti-diarrhea treatment, where colitis is not suspected. |
| Hepatic | Grade 3 ALT or AST in the absence of significantly increased |
| | bilirubin that resolves to ≤ Grade 1 within 7 days. |
| Amylase and lipase | Asymptomatic Grade ≥ 3 amylase or lipase. |
| Dermatologic | Grade 3 non bullous rash without epidermal detachment that |
| | resolves to ≤ Grade 1 within 7 days of starting treatment. |
| Hematology | Grade 3 neutropenia without fever or other clinical symptoms |
| | that resolves to ≤ Grade 1 within 7 days. |
| | Grade 3 thrombocytopenia without clinically significant bleeding. |
| | Grade 3 anemia that resolves within 7 days in the absence of transfusion. |
| | Lymphopenia of any grade is not a DLT. |
| Electrolytes | Grade 3 electrolyte abnormalities that resolve to ≤ Grade 1 within |
| | 7 days after starting supplementation |
| Musculoskeletal | Grade 3 asymptomatic increase in creatine kinase that resolves within |
| | 14 days in the absence of evidence of cardiac involvement. |
| Immune-related | In general, a Grade 3 immune-related AE that resolves to ≤ Grade 1 |
| toxicities* | within 7 days of starting appropriate treatment is not a DLT. |

The following Grade 2 AEs related to study treatment are considered DLTs:

| | |
|---|---|
| Ocular disorders | Grade 2 eye pain or reduction of visual acuity are DLTs if they do not |
| | respond to topical therapy and do not improve to Grade 1 severity |
| | within 2 weeks of the initiation of topical therapy, OR if they require |
| | systemic treatment. |
| Pneumonitis | Grade 2 pneumonitis is a DLT if it does not resolve to ≤ Grade 1 |
| | within 7 days of starting corticosteroids. |
| Myocarditis | Grade 2 myocarditis is a DLT. |
| Colitis | Grade 2 colitis is a DLT if it persists > 7 days despite treatment |
| | with corticosteroids. |
| Hepatic | Grade 2 ALT or AST accompanied by bilirubin > 1.5 × ULN is a DST. |
| Dermatologic | Grade 2 bullous disease that does not resolve to ≤ Grade 1 within |
| | 7 days of starting corticosteroids is a DLT. |
| Other adverse events | Other clinically significant toxicities, including a single event or multiple |
| | occurrences of the same event that lead to a dosing delay of > 14 days) in |
| | cycle 1 may be considered to be DLTs by the Investigators and Novartis, |
| | even if not CTCAE Grade 3 or higher. |

*Depending on the nature of the AE, there may be cases where immune-related Grade 2-3 AEs of any duration warrant declaration of a DLT and permanent study discontinuation (e.g. Steven Johnson Syndrome (SJS)). DLT determination not already outlined in this table will be made on a case-by case basis after Investigator discussion with the Novartis Medical Monitor.

Biomarkers

Biomarker analyses will be used to investigate the effect of CHO hetIL-15 as a single agent or in combination with anti-PD-1 antibody at the molecular and cellular level as well as to determine how changes in the markers may relate to exposure and clinical outcomes. In addition, potential predictive markers of efficacy, as well as mechanisms of resistance to hetIL-15 will also be explored.

TABLE 1
Biomarker sample collection plan (single agent group)

| | | Approximate | | |
|---|---|---|---|---|
| Sample Type | Visit/Time Points | volume | Marker | Purpose |
| Tumor samples | | | | |
| Newly Obtained Tumor | Screening**, | 4-6 passes of | Characterization of | Assess potential |
| biopsy** | C1D21-D28, | Core Needle | tumor | biomarkers of |
| | C3D21-D28 (if the | Biopsy | microenvironment, | response/resistance |
| | patient has started | | assessment of T-cells | |
| | anti-PD-1 antibody | | and NK cells infiltrates. | |
| | EOT (optional) | | Expression of markers | |
| | | | including but not | |
| | | | limited to: CD8, PDL1, | |
| | | | CD56, Granzyme B. | |
| | | | Genomic/transcriptomic | |
| | | | analysis of immune and | |
| | | | cancer related genes | |
| | | | and clonal abundance | |
| | | | of T-cells. | |
| Blood samples | | | | |
| Blood sample for | C1D1 (pre-dose) | 10 mL | Phenotypic | Pharmacodynamic |
| immunomonitoring | C1D3 | | characterization of | effect |
| | C1D4 | | activated immune cells | |
| | C1D8 (pre-dose) | | (e.g. CD8) | |
| | C1D15 (pre-dose) | | | |
| | C1D17 | | | |
| | C2D1 (pre-dose) | | | |
| | C2D3 | | | |
| | C2D4 | | | |
| | C2D8 (pre-dose) | | | |
| | C2D15 (pre-dose) | | | |
| | C2D17 | | | |
| | C3D1 (pre-dose) | | | |
| | C3D3 | | | |
| | C3D4 | | | |
| | C3D8 (pre-dose) | | | |
| | C3D15 | | | |
| | C3D17 | | | |
| | C4D1 (pre-dose) | | | |
| | C4D3 | | | |
| | C4D4 | | | |
| | C4D8 (pre-dose) | | | |
| | C4D15 | | | |
| | C4D17 | | | |
| Blood sample for soluble | C1D1 (pre-dose) | 3 mL | Cytokine analysis (e.g. | Characterize circulating |
| cytokines | C1D1 (8 hrs (±1) | | IFN-γ, IL-2, IL-4, IL-6, | levels of cytokines |
| | post-dose) | | IL-8, IL-10, TNF-α) | |
| | C1D2 | | | |
| | C1D3 | | | |
| | C1D4 | | | |
| | C1D8 (pre-dose) | | | |
| | C1D15 (pre-dose) | | | |
| | C1D17 | | | |
| | C3D1 (pre-dose) | | | |
| | C3D1 (8 hrs post) | | | |
| | C3D3 | | | |
| | C3D4 | | | |
| | C3D8 (pre-dose) | | | |
| | C3D15 (pre-dose) | | | |
| | C3D17 | | | |
| Blood sample for cfDNA | C1D1 (Pre-dose), | 10 mL | DNA sequencing in cell | Analysis of tumor |
| | EOT | | free DNA | mutational burden and |
| | | | | other genetic alterations |
| | | | | as potential biomarkers |
| | | | | of response/resistance |

TABLE 2
Biomarker sample collection plan (CHO hetIL-15 and
anti-PD-1 antibdy combination from C1D1)

| | | Approximate | | |
|---|---|---|---|---|
| Sample Type | Visit/Time Points | volume | Marker | Purpose |
| Tumor samples | | | | |
| Newly Obtained Tumor | Screening**, | 4-6 passes of | Characterization of | Assess potential |
| biopsy** | C1D21-D28 | Core Needle | tumor | biomarkers of |
| | EOT (mandatory if | Biopsy | microenvironment, | response/resistance |
| | safe and medically | | assessment of | |
| | feasible) | | lymphocyte | |
| | | | infiltrates. | |
| | | | Expression of | |
| | | | markers such as: | |
| | | | CD8, PDL1, CD56, | |
| | | | Granzyme B | |
| | | | Whole genomic | |
| | | | analysis of immune | |
| | | | and cancer related | |
| | | | genes and clonal | |
| | | | abundance of T- | |
| | | | cells. | |
| Blood samples | | | | |
| Blood sample for | C1D1 pre-dose | 10 mL | Phenotypic | Pharmacodynamic effect |
| immunomonitoring | C1D3 | | characterization of | |
| | C1D4 | | activated immune | |
| | C1D8 (pre-dose) | | cells (e.g. CD8) | |
| | C1D15 (pre-dose) | | | |
| | C1D17 | | | |
| | C2D1 (pre-dose) | | | |
| | C2D3 | | | |
| | C2D4 | | | |
| | C2D8 (pre-dose) | | | |
| | C2D15 (pre-dose) | | | |
| | C2D17 | | | |
| Blood sample for soluble | C1D1 (pre-dose) | 3 mL | Cytokine analysis | Characterize circulating |
| cytokines | C1D1 (8 hrs (±1) | | (e.g. IFN-γ, IL-2, IL- | levels of cytokines |
| | post-dose) | | 4, IL-6, IL-8, IL-10, | |
| | C1D2 | | TNF-α) | |
| | C1D3 | | | |
| | C1D4 | | | |
| | C1D8 (pre-dose) | | | |
| | C1D15 (pre-dose) | | | |
| | C1D17 | | | |
| Blood sample for cfDNA | C1D1 (Pre-dose), | 10 mL | DNA sequencing in | Analysis of tumor |
| | EOT | | cell free DNA | mutational burden and |
| | | | | other genetic alterations |
| | | | | as potential biomarkers |
| | | | | of response/resistance |

It is expected that CHO hetIL-15 will promote NK and CD8+ T cells and increase antitumore immunity, and the CHO hetIL-15 and anti-PD-1 antibody combination therapy will inhibits tumor growth.

Example 6 hetIL-15 produced by CHO cell does not have IL-15Rα chain C-terminal splicing variant compared to hetIL-15 produced by HEK293.

Heterogeneity by size was compared for HEK293 and CHO hetIL-15 by peptide mapping, SDS-PAGE (native and reducing conditions), SEC, RP-HPLC and mass spectrometry.

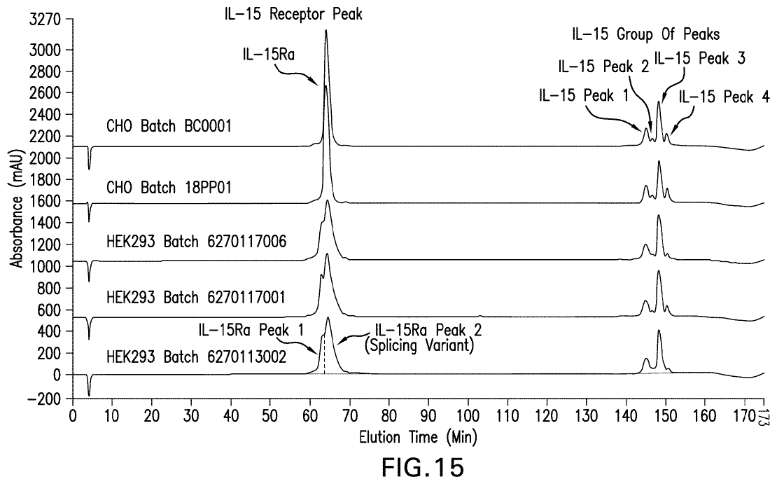

As shown on FIG. 15, chromatographic profiles of HEK293 batches and CHO batches are significantly different. HEK293 batches exhibit a double IL-15Rα peak due to the presence of a splicing variant which is absent in CHO batches. The location of the splicing variant is in the C-terminal region of the IL-15 receptor peak. HEK293 and CHO batches are very similar with respect to distribution of the four IL-15 peaks and peak area ratio of IL-15Rα and IL-15. The peak area ratio monitors the stoichiometry of IL-15Rα and IL-15: a ratio of 2.0 corresponds to a 1:1 molar ratio of the two chains.

The splicing variant of the IL-15Rα chain was detected in HEK293 batches only, consisting of 159 residues (I1-G159) (shown below), which was determined by peptide mapping:

ITCPPMSVEHADIWVKSYSLYSRERYICNSGFKRKAGTSSLTE
CVLNKATNVAHWTTPSLKCIRDPALVHQRPAPPSTVTTAGVTP
QPESLSPSGKEPAASSPSSNNTAATTAATVPGSQLMPSKSPST
GTTBISSHESSHGTPSQTTAKNWELADIG

It is understood that the examples and aspects described herein are for illustrative purposes only and that various modifications or changes in light thereof will be suggested to persons skilled in the art and are to be included within the spirit and purview of this application and scope of the appended claims.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety (or as context dictates), to the same extent as if each were incorporated by reference individually. In case of conflict, the present specification, including definitions, will control.

What is claimed is:

1. A polypeptide complex comprising a human interleukin 15 (IL-15) polypeptide and a human interleukin 15 receptor alpha (IL-15Rα) polypeptide, wherein the polypeptide complex comprises N-linked glycans comprising FA2G2, FA2G2S1, FA2G2S2, FA3G3S1, FA2F1G2S2, FA3G2S2, and FA3G3S3.

2. The polypeptide complex of claim 1, wherein IL-15 polypeptide has the sequence of SEQ ID NO: 1 or 5, and IL-15Rα has the sequence of SEQ ID NO: 6, 7, 10, 12, 14 or 21.

3. The polypeptide complex of claim 1, wherein the N-linked glycans comprise at least 10%, 12.5%, 15%, 17.5%, 20% or 22.5% of FA2G2S1.

4. The polypeptide complex of claim 1, wherein the N-linked glycans comprise at least 10%, 20%, 30%, or 40% of FA2G2S2.

5. A polypeptide complex comprising a human interleukin 15 (IL-15) polypeptide and a human interleukin 15 receptor alpha (IL-15Rα) polypeptide, wherein the polypeptide complex comprises O-linked glycans, and wherein at least 80%, 85%, 90% or 95% of the glycans is core-1 O-linked glycan.

6. The polypeptide complex of claim 5, wherein IL-15 polypeptide has the sequence of SEQ ID NO: 1 or 5, and IL-15Rα has the sequence of SEQ ID NO: 6, 7, 10, 12, 14 or 21.

7. The polypeptide complex of claim 5, wherein the core-1 O-linked glycan is monosialylated and/or disialylated.

8. A polypeptide complex comprising a human interleukin 15 (IL-15) polypeptide and a human interleukin 15 receptor alpha (IL-15Rα) polypeptide,
wherein the polypeptide complex comprises O-linked glycans, and wherein at least 80%, 85%, 90% or 95% of the glycans has core-1 O-linked glycan structures; and
wherein the polypeptide complex comprises N-linked glycans comprising FA2G2, FA2G2S1, FA2G2S2, FA3G3S1, FA2F1G2S2, FA3G2S2, and FA3G3S3.

9. The polypeptide complex of claim 8, wherein IL-15 polypeptide has the sequence of SEQ ID NO: 1 or 5, and IL-15Rα has the sequence of SEQ ID NO: 6, 7, 10, 12, 14 or 21.

10. The polypeptide complex of claim 8, wherein the N-linked glycans comprise at least 10%, 12.5%, 15%, 17.5%, 20% or 22.5% of FA2G2S1.

11. The polypeptide complex of claim 8, wherein the N-linked glycans comprise at least 10%, 20%, 30%, or 40% of FA2G2S2.

12. The polypeptide complex of claim 8, wherein the core-1 O-linked glycan is predominantly monosialylated and/or disialylated.

13. An isolated IL-15/IL-15Rα heterodimer produced in a non-human cell, wherein the IL-15/IL-15Rα heterodimer comprises α(2,6) O-linked sialylation.

14. The isolated IL-15/IL-15Rα heterodimer of claim 13, wherein the non-human cell is a recombinant Chinese hamster ovary (CHO) cell.

15. The isolated IL-15/IL-15Rα heterodimer of claim 14, wherein the CHO cell is altered to impair the function of matriptase.

16. The isolated IL-15/IL-15Rα heterodimer of claim 13, wherein the isolated IL-15/IL-15Rα heterodimer comprises O-linked glycans, and wherein at least 80%, 85%, 90% or 95% of the glycans is core-1 O-linked glycan.

17. The isolated IL-15/IL-15Rα heterodimer of claim 16, wherein about 15% of the 0-glycans has α(2,6)-linked sialylation.

18. A pharmaceutical composition comprising the polypeptide complex or the isolated IL-15/IL-15Rα heterodimer of claim 13.

19. The pharmaceutical composition of claim 18, further comprising a pharmaceutically acceptable carrier.

20. A non-human cell comprising nucleic acids encoding a human interleukin 15 (IL-15) polypeptide and a human interleukin 15 receptor alpha (IL-15Rα) polypeptide, wherein the IL-15 and IL-15 Rα expressed by the cell form a heterodimer, and wherein the heterodimer comprises α(2,6)-linked sialylation.

21. The non-human cell of claim 20, wherein the non-human cell is a recombinant Chinese hamster ovary (CHO) cell.

22. The non-human cell of claim 20, wherein the CHO cell is altered to impair the function of matriptase.

23. A method of treating cancer, comprising administering to a subject in need of the pharmaceutical composition of claim 18.

24. A method of producing cells that express IL-15/IL-15a heterodimer, comprising:
(a) providing non-human cells;
(b) transfecting the non-human cells with two vectors at the same time, wherein the two vectors comprises a first vector encoding both IL-15Rα and IL-15, and a second vector encoding a portion of IL-15Rα and culturing the transfected cells;
(c) transfecting the cells from step b) with a third vector encoding IL-15 and culturing the transfected cells; and
(d) isolating individual clones that express IL-15/IL-15a heterodimer.

25. The method of claim 24, wherein the non-human cell is a recombinant Chinese hamster ovary (CHO) cell.

26. The method of claim 25, wherein the CHO cell is modified to impair the function of the matriptase gene.

27. The method of claim 24, wherein the IL-15Rα has the sequence of SEQ ID NO: 6, 12 or 14.

28. The method of claim 24, wherein the IL-15 has the sequence of SEQ ID NO: 1 or 5.

29. The method of claim 24, wherein the portion of IL-15 Rα is a soluble portion of IL-15Rα.

30. The method of claim 29, wherein the soluble portion of IL-15Rα has the sequence of SEQ ID NO:7, 10 or 21.

31. A method of producing IL-15/IL-15 Rα heterodimer, comprising
(a) culturing the cells produced by claim 24 under a condition that allow for expression of a IL-15/IL-15Rα heterodimer and secretion of the IL-15/IL-15Rα heterodimer, and
(b) isolating the IL-15/IL-15a heterodimer from the cell culture.

32. A polypeptide complex comprising a human interleukin 15 (IL-15) polypeptide and a human interleukin 15 receptor alpha (IL-15Rα) polypeptide, wherein the polypeptide complex is produced by a recombinant Chinese hamster ovary (CHO) cell, and wherein the polypeptide complex does not have a IL-15Rα chain splicing variant.

33. The polypeptide complex according to claim 32, wherein the CHO cell is altered to impair the function of matriptase.

34. The polypeptide complex of claim 32, wherein the IL-15Rα chain splicing variant comprise 159 residues spanning from I1 to G159.
